SEQUENCE LISTING (157 sequences)

SEQ ID NO: 1 - DNA, 77 bp, synthetic construct
  source 1..77: mol_type = other DNA
gcgtgattgatggaattggcatacataccgtcctcgaaagtcgggttttgagggttcgaatcccttgtcacgcacca

SEQ ID NO: 2 - DNA, 77 bp, synthetic construct
  source 1..77: mol_type = other DNA
gcgtgattgatggaattggcatacataccgtccttgaaagtcgggttttgagggttcgaatcccttgtcacgcacca

SEQ ID NO: 3 - DNA, 77 bp, synthetic construct
  source 1..77: mol_type = other DNA
gggggtgtaatcgaattggcataggtactggactcgaaattcaggttttgtgggttcgaatcccaccacccctacca

SEQ ID NO: 4 - DNA, 77 bp, synthetic construct
  source 1..77: mol_type = other DNA
gggggtgtaatcgaattggcataggtactggacttgaaattcaggttttgtgggttcgaatcccaccacccctacca

SEQ ID NO: 5 - DNA, 77 bp, synthetic construct
  source 1..77: mol_type = other DNA
gcacctatagcccaactggtagaggcagcagactcgaaatctgctcagtgtgagttcgaatctctctaggtgtacca

SEQ ID NO: 6 - DNA, 77 bp, synthetic construct
  source 1..77: mol_type = other DNA
gcacctatagcccaactggtagaggcagcagacttgaaatctgctcagtgtgagttcgaatctctctaggtgtacca

SEQ ID NO: 7 - RNA, 59 nt, synthetic construct
  source 1..59: mol_type = other RNA
ctttgagcctaatgcctgaaagatcggaagagcacacgtctagttctacagtccgacga

SEQ ID NO: 8 - DNA, 61 bp, synthetic construct
  source 1..61: mol_type = other DNA
atcgtcggactgtagaactagacgtgtgctcttccgatctttcaggcattaggctcaaagn

SEQ ID NO: 9 - DNA, 27 bp, synthetic construct
  source 1..27: mol_type = other DNA
gatcnnnagatcggaagagcgtcgtgt

SEQ ID NO: 10 - protein, 27 aa, synthetic construct
MSKGPGKVPGAGVPGXGVPGVGKGGGT

SEQ ID NO: 11 - DNA, 87 bp, synthetic construct
  source 1..87: mol_type = other DNA
gccgaggtggtggaattggtagacacgctacctttgaaaggtagtgcccaatagggcttacgggttcaagtcccgtcctcggtacca

SEQ ID NO: 12 - DNA, 87 bp, synthetic construct
  source 1..87: mol_type = other DNA
gccgaggtggtggaattggtagacacgctaccgttgaatggtagtgcccaatagggcttacgggttcaagtcccgtcctcggtacca

SEQ ID NO: 13 - DNA, 89 bp, synthetic construct
  source 1..89: mol_type = other DNA
gccgaggtggtggaattggtagacacgctaccccccgaaaggtagtgcccaatagggcttacgggttcaagtccttaccctcggtacca

SEQ ID NO: 14 - DNA, 87 bp, synthetic construct
  source 1..87: mol_type = other DNA
gccgaggtggtggaattggtagacacgctaccttcgaatggtagtgcccaatagggcttacgggttcaagtcccgtcctcggtacca

SEQ ID NO: 15 - DNA, 74 bp, synthetic construct
  source 1..74: mol_type = other DNA
gccagagtagctcaattggtagagcaacaggtcaccgatcctggtggttctcggttcaagtccgagctctggtc

SEQ ID NO: 16 - DNA, 74 bp, synthetic construct
  source 1..74: mol_type = other DNA
gccagggtagctcaatcggtagagcagcggttcctggagtccgctggttctcggttcaagcccgagccctggtt

SEQ ID NO: 17 - DNA, 74 bp, synthetic construct
  source 1..74: mol_type = other DNA
gtcggggtagctcaaatggtagagtggcaggccaacatagccagcagatctcggttcaaacccgagccctgacc

SEQ ID NO: 18 - DNA, 73 bp, synthetic construct
  source 1..73: mol_type = other DNA
gtcggggtagctcaattggtagagcggcaggctcatcccctgcaggttctcggttcaatcccgggtcccgacg

SEQ ID NO: 19 - DNA, 72 bp, synthetic construct
  source 1..72: mol_type = other DNA
gccagggtagctcaattggtagagcatcaggctagtatcctgtcggttccggttcaagtccgggccctggtt

SEQ ID NO: 20 - DNA, 73 bp, synthetic construct
  source 1..73: mol_type = other DNA
gccagcgtagctcaattgttagagcagcggccaccaagcctgcaggttctcggttcaagtccgggcgctggca

SEQ ID NO: 21 - DNA, 84 bp, synthetic construct
  source 1..84: mol_type = other DNA
gccagggtagctcaattggtagagcggcagacaccacctacgtggtctagtctgtggatctcggttcaagtccgagtcctggcc

SEQ ID NO: 22 - DNA, 74 bp, synthetic construct
  source 1..74: mol_type = other DNA
accagagtggctcacctggtagagcaccaggctgcccatcctgttggttctcggttcaaatccgagctctggtg

SEQ ID NO: 23 - DNA, 73 bp, synthetic construct
  source 1..73: mol_type = other DNA
gctccagtagcgcaatcggcagcgcgtggtacttataatgccaaggttgtgagttcgatcctcacctggagca

SEQ ID NO: 24 - DNA, 72 bp, synthetic construct
  source 1..72: mol_type = other DNA
tcccttatagctcagttggtagagactttgcctgcagtgcaggagacccaggttcgattcctgggttgggaa

SEQ ID NO: 25 - DNA, 74 bp, synthetic construct
  source 1..74: mol_type = other DNA
ttccatggtggctcaaaggttaaagcatctgcctgcaatgcgggagacccatgttcaatccctgagtgaggaag

SEQ ID NO: 26 - DNA, 72 bp, synthetic construct
  source 1..72: mol_type = other DNA
ggtggtgtagcttagttgttagagcgtcggtgcatcacaccggagaccccagatcgaaactgggcatcacca

SEQ ID NO: 27 - DNA, 73 bp, synthetic construct
  source 1..73: mol_type = other DNA
gacagtgtagctcagccgtaagagcgtcggacttgcactccggaggtccccagatcgaaactgggtactgccg

SEQ ID NO: 28 - DNA, 75 bp, synthetic construct
  source 1..75: mol_type = other DNA
gccaagacatcatccagtcggtagagcatgggactcttaatcccagggtcgtgggttcgtgccccacgttgggcg

SEQ ID NO: 29 - DNA, 92 bp, synthetic construct
  source 1..92: mol_type = other DNA
ggcggtgtagctcaacggcagagcgccggccccgtccccgcgctccccgggttcgggaccggagggaccgagatcgaccctcggcatcgccg

SEQ ID NO: 30 - DNA, 73 bp, synthetic construct
  source 1..73: mol_type = other DNA
ggcggtatagctcagctgtcagagcgtcggacctaaaatccggaggaccccagatcaaaactgggtatcgccg

SEQ ID NO: 31 - DNA, 73 bp, synthetic construct
  source 1..73: mol_type = other DNA
ggcggcatagctcaggtggcagagcgtcggactaacagttcggaggtccccagatcgaaactgggtgcagtcg

SEQ ID NO: 32 - DNA, 73 bp, synthetic construct
  source 1..73: mol_type = other DNA
ggcggtgtagctcagccgtaagagcgtcgggtcaaaagtccggaggtccccagatcgaaactgggtatcgccg

SEQ ID NO: 33 - DNA, 76 bp, synthetic construct
  source 1..76: mol_type = other DNA
tccctcgttagttcagtggtagaatacccgcctcctctccgggcgggagaccgaggttcgattcctcgacgaggaa

SEQ ID NO: 34 - DNA, 99 bp, synthetic construct
  source 1..99: mol_type = other DNA
gtcccgatagtacaacggctagtatgcccgcaccgcccggatgccgctctggcggcggcggccgtgcgggggaccggggttcgactccccgacgggacg

SEQ ID NO: 35 - DNA, 92 bp, synthetic construct
  source 1..92: mol_type = other DNA
tcctcgttagtatagcggctagtacgcccgcggccgacccggcatacagataccggccgcgggagaccggggttcgagtccccgacgagggt

SEQ ID NO: 36 - DNA, 92 bp, synthetic construct
  source 1..92: mol_type = other DNA
tcctcgttagtatagcggttagtacgcccgcaacactaacccgcccccatttgcacaagcgggagaccggggttcgaatccccgacgaggaa

SEQ ID NO: 37 - DNA, 80 bp, synthetic construct
  source 1..80: mol_type = other DNA
tcctcgttagtttagaggtagaacgcccatcgtcttcgcgaggagaatgggagaccggggttcgagtccccgacggggat

SEQ ID NO: 38 - DNA, 72 bp, synthetic construct
  source 1..72: mol_type = other DNA
tcctcgttagtatagtggttagtattcccgtctgtcgcacgggagaccggggttcgattccccgacgaggag

SEQ ID NO: 39 - DNA, 74 bp, synthetic construct
  source 1..74: mol_type = other DNA
gtccccgtttgtacattggtagtactcccgctcgaggtgctgggattgcggggttcgagtccccggcggggaca

SEQ ID NO: 40 - DNA, 77 bp, synthetic construct
  source 1..77: mol_type = other DNA
tcctcgttagtatagtggtagtacaaccggttcgcgcgggcggccgggagaccgaggttcgattcctcggcgaggac

SEQ ID NO: 41 - DNA, 77 bp, synthetic construct
  source 1..77: mol_type = other DNA
tccccgttagtttagtggtataacacccatctcaccttggtagacgggagaccggggttcgattccccgacggggac

SEQ ID NO: 42 - DNA, 78 bp, synthetic construct
  source 1..78: mol_type = other DNA
tcctcgttagtatagtggcagaacacccatctcaaggcctccggatgggagaccggggttcgattccccgacgagggt

SEQ ID NO: 43 - DNA, 76 bp, synthetic construct
  source 1..76: mol_type = other DNA
tcctcgttagtacagtggcagtacgcagcctgtcaacgcagacgggcaaaccggggttcgattccccgacgaggat

SEQ ID NO: 44 - DNA, 80 bp, synthetic construct
  source 1..80: mol_type = other DNA
tcctcggtagtacaacggttactacgcccgtatatatatagagtaaacgggagatcggggttcgactccccgccgaggaa

SEQ ID NO: 45 - DNA, 71 bp, synthetic construct
  source 1..71: mol_type = other DNA
tcctcgttagtatagtggtagtatgcccgtctgtcgcacgggagaccggggttcgattccccgacgaggag

SEQ ID NO: 46 - DNA, 76 bp, synthetic construct
  source 1..76: mol_type = other DNA
ccctcgttagtctagcggcaaaacgcccagtctggaatgtcgatgggagtctggggttcgattccccaacgagggt

SEQ ID NO: 47 - DNA, 80 bp, synthetic construct
  source 1..80: mol_type = other DNA
ccctcattagtacagcggttagtacacccggacgctccacggacacacgggagagcggggttcgactccccgatgagggc

SEQ ID NO: 48 - DNA, 81 bp, synthetic construct
  source 1..81: mol_type = other DNA
tcctcgttagtacagcggttagtatacgcacttgactgtttgtcaggtagcgagagcggggttcgactccccggcggggat

SEQ ID NO: 49 - DNA, 81 bp, synthetic construct
  source 1..81: mol_type = other DNA
gcctcgctagtacaggggttagtatgcccgtatggtatatgtgtcacgcgggagaccggggttcgactccccggcgaggcg

SEQ ID NO: 50 - DNA, 81 bp, synthetic construct
  source 1..81: mol_type = other DNA
cccttgttagtacagcggttagtataccagtacggcccaacaaccgtgctggagaccggggttcgactccccgacgagggt

SEQ ID NO: 51 - DNA, 74 bp, synthetic construct
  source 1..74: mol_type = other DNA
tccccgttagtacagtggcagtacgccgtccgtggagcggatggacaaccggggttcgattccccgacggggca

SEQ ID NO: 52 - DNA, 85 bp, synthetic construct
  source 1..85: mol_type = other DNA
tcctcgttagtatagcggttaatacacccgccgaaaggctttagacctttgacgggaaagtggggttcgactccccgacgaggat

SEQ ID NO: 53 - DNA, 80 bp, synthetic construct
  source 1..80: mol_type = other DNA
tcctcgttagtacagcggttagtacgccaacccaatagctatctgggttggaaaccggggttcaactccccgacgaggat

SEQ ID NO: 54 - DNA, 76 bp, synthetic construct
  source 1..76: mol_type = other DNA
ccctcgctagtatagtggttattacgctcggcggtgcctaggccgagagaccggggttcgatcccccggcgaggga

SEQ ID NO: 55 - DNA, 81 bp, synthetic construct
  source 1..81: mol_type = other DNA
tccccattagtacagcggttagtacactcgcatgagtcatacctgatgcgagagaccggggttcgactccccgatggggga

SEQ ID NO: 56 - DNA, 72 bp, synthetic construct
  source 1..72: mol_type = other DNA
tcctcgttagtatagtggttaatatccccgtttgtcgcacgggagaccggggttcgaatccccgacgaggag

SEQ ID NO: 57 - DNA, 73 bp, synthetic construct
  source 1..73: mol_type = other DNA
gctgaagtagctcagttgggagagcattagactgaagatctaaaggtccctggttcaaccccgggcttcagca

SEQ ID NO: 58 - DNA, 74 bp, synthetic construct
  source 1..74: mol_type = other DNA
gctctagtggcgcagttaggtagcgcgcagtgcttataacactgaggtcgtgagttcgatcctcacctagagca

SEQ ID NO: 59 - DNA, 82 bp, synthetic construct
  source 1..82: mol_type = other DNA
tcctcggtagtacaacggttactacgcccgtatatatatatagagtaaacgggagatcggggttcgactccccgccgaggaa

SEQ ID NO: 60 - DNA, 74 bp, synthetic construct
  source 1..74: mol_type = other DNA
tccccgttagtacagtggcagtacgccgtccgtggagcggatggacaaccggggctcgattccccgacggggca

SEQ ID NO: 61 - DNA, 81 bp, synthetic construct
  source 1..81: mol_type = other DNA
tttgggggttggggatttagctcagtgatagagctcttgcctagcacgcaagccctgggttcggtccccccagctctgaaa

SEQ ID NO: 62 - DNA, 73 bp, synthetic construct
  source 1..73: mol_type = other DNA
ggccagataccgtaggggttacacaaccaggttgtaacacttgggtgtctgggttcgatttccagtctggccg

SEQ ID NO: 63 - DNA, 73 bp, synthetic construct
  source 1..73: mol_type = other DNA
gccgagatagctcagtagggagagcgtcggactgaagttctgaaggtccctggttcgatcccaggtttcggca

SEQ ID NO: 64 - DNA, 87 bp, synthetic construct
  source 1..87: mol_type = other DNA
atctatgttggcttagtggttactaggcagggagataagcgaggttttcaagccacctactcctgggttcgaaccccagcatagatt

SEQ ID NO: 65 - DNA, 74 bp, synthetic construct
  source 1..74: mol_type = other DNA
gccagggtagctcaatcggtagagtagcggtccctggagtccgctggttctcggttcaagcccgagccctggtt

SEQ ID NO: 66 - DNA, 74 bp, synthetic construct
  source 1..74: mol_type = other DNA
gtcggggtagctcaaatggtagagtggcaggccaacatagccagcagatctcggttcaaacccgagctctgacc

SEQ ID NO: 67 - DNA, 73 bp, synthetic construct
  source 1..73: mol_type = other DNA
gtcggggtagctcaattggtagagcggcaggctcatcccctgcaggttctcggttcgatcccgggtcccggcg

SEQ ID NO: 68 - DNA, 72 bp, synthetic construct
  source 1..72: mol_type = other DNA
gccagggtagctcaagtggtagagcatcaggctagtaccctgtcggttccggttcaagtccgggccctggtt

SEQ ID NO: 69 - DNA, 84 bp, synthetic construct
  source 1..84: mol_type = other DNA
gccagggtagctcaattggtagagcgacagccaccacctacgtggtctagtctgtggatctcggttcaagtccgagtcctggcc

SEQ ID NO: 70 - DNA, 74 bp, synthetic construct
  source 1..74: mol_type = other DNA
accagagtggctcacctggtagagcaccaggctgcccatcctgttggttctcggttcaagtcccagctctggtg

SEQ ID NO: 71 - DNA, 86 bp, synthetic construct
  source 1..86: mol_type = other DNA
gggctcgtagcttagccaggtagagcaacgggctcttaacctagggagatacccgtaggtcccgggttcgaatcccggcgagcccg

SEQ ID NO: 72 - DNA, 111 bp, synthetic construct
  source 1..111: mol_type = other DNA
tgcgtaaaatggcaacttctttcaattcttcagcacgatcacgtagctcatccagttcacggcgggcttttaacccgtaggtcccgggttcgaatcccggcgggcccgcca

SEQ ID NO: 73 - DNA, 76 bp, synthetic construct
  source 1..76: mol_type = other DNA
acggtaggctgggcgccccgcctactgcgggcctttcgagcccgtgacccgggttcaaatcccggccgcggcacca

SEQ ID NO: 74 - DNA, 63 bp, synthetic construct
  source 1..63: mol_type = other DNA
gggggcgggcttaagacccgttggcgtaggcctgcgtgggttcaaatcccaccccccgcacca

SEQ ID NO: 75 - DNA, 128 bp, synthetic construct
  source 1..128: mol_type = other DNA
ggtgtaggttatgagagtggttgagaggcttggagctccacacagctatacgcctgctaaacacgcctacaccgggccccgcccttacacggcggaggtccggggttcaaatccccgcgggcccacca

SEQ ID NO: 76 - DNA, 106 bp, synthetic construct
  source 1..106: mol_type = other DNA
gggcaggtgagtcgagcggccgaaactcccgggactccggggtaatgcggcgaggcgacctaggcgctgtcgacccgtaccgcgggttcaaatcccgccctgcccg

SEQ ID NO: 77 - DNA, 119 bp, synthetic construct
  source 1..119: mol_type = other DNA
gggaaggtagctcaatggtagagccaaagcgcgtattgaacctcccaactacgggggtgacgcgacggtgacacgtaagtacctcaccggggatgttggttcgaatccaaccctttccg

SEQ ID NO: 78 - DNA, 75 bp, synthetic construct
  source 1..75: mol_type = other DNA
agtcccctggggtagtttggccaatcctctgggcctttggagctcaggacgcgagttcaaatctcgcggggacaa

SEQ ID NO: 79 - DNA, 75 bp, synthetic construct
  source 1..75: mol_type = other DNA
agtcccctggggtagtttggccaatcctcagcgcctttggagcgctggacgcgagttcaaatctcgcggggacaa

SEQ ID NO: 80 - DNA, 132 bp, synthetic construct
  source 1..132: mol_type = other DNA
agccccgtggccgaatggttcaaggcgctattacgtcgcttctgagatttcagccacgttttgtggagcgattggaagcgttacttcgctatctggaagcagagattgtaggttcgaatcctaccggggcta

SEQ ID NO: 81 - DNA, 72 bp, synthetic construct
  source 1..72: mol_type = other DNA
gggcgattggtctactggatatgacacatccctgcgaaggatgagaagggagttcgattctctcattgccca

SEQ ID NO: 82 - DNA, 71 bp, synthetic construct
  source 1..71: mol_type = other DNA
gccgaagtggtctaaaagtaagatgcgtgcctgtcacgcacgagactcgggagcattacccggcttcggcg

SEQ ID NO: 83 - DNA, 88 bp, synthetic construct
  source 1..88: mol_type = other DNA
gctggcttggggtagtggacgcacggcattaagccaatccttcgaccctttcgaggtcgagacacgagttcgaatctcgtagccagta

SEQ ID NO: 84 - DNA, 74 bp, synthetic construct
  source 1..74: mol_type = other DNA
gggtcgataggataatttgggaatccacctgtttgaagaacaggcgcatctcggttcaaatccgggtcggccca

SEQ ID NO: 85 - DNA, 76 bp, synthetic construct
  source 1..76: mol_type = other DNA
gctgatggagtctccgctggcgagagacgctacgttctcagcgtagaggtcgcgggttcaattcctgtcatcagca

SEQ ID NO: 86 - DNA, 71 bp, synthetic construct
  source 1..71: mol_type = other DNA
gcgtggctgttccaatggcaagatgcgggccttccaagcctgagatacgagttcgactctcgtgccgcgca

SEQ ID NO: 87 - DNA, 71 bp, synthetic construct
  source 1..71: mol_type = other DNA
gcgtggttggtccaatggaaagacggctccctgccacggagcagactcgggttcgattcccgaatcgcgca

SEQ ID NO: 88 - DNA, 73 bp, synthetic construct
  source 1..73: mol_type = other DNA
gtcgcggtagttcaattagtagaacactcggctgttacccgagatgttggaggtgcaagtcctccctgcgacg

SEQ ID NO: 89 - DNA, 73 bp, synthetic construct
  source 1..73: mol_type = other DNA
ctctcgaagacccgctggtaagggtacacgcctgtagagcgtgttgcagtaggttcgaatcctactcgggaga

SEQ ID NO: 90 - DNA, 76 bp, synthetic construct
  source 1..76: mol_type = other DNA
gggctgatggcgaaacttggctaaacgcggaaggctcttgaccttctacatacgggttcaaatcctgttcagccca

SEQ ID NO: 91 - DNA, 86 bp, synthetic construct
  source 1..86: mol_type = other DNA
cccgacgttgagcagtctggctatgctcggcaggctcatatcctgttgtgcttcgcacgaaatgggttcgaatcccatcgtcggaa

SEQ ID NO: 92 - DNA, 103 bp, synthetic construct
  source 1..103: mol_type = other DNA
gggccgctggtctaatggcaagacacttgctttgcaagagttgaagttacggcttctgctccaaattagcaaggaactcccggttcgaatccggggcggtcca

SEQ ID NO: 93 - DNA, 74 bp, synthetic construct
  source 1..74: mol_type = other DNA
tccgttgtggcccaatttggacgggcactcgcctcgtaagcgagaggttgtgggttcgattcccaccagcggat

SEQ ID NO: 94 - DNA, 71 bp, synthetic construct
  source 1..71: mol_type = other DNA
tccagtatggtctaatggtagggcacggctttggtatggctgacgtgagggttcaactccctctgctggat

SEQ ID NO: 95 - DNA, 73 bp, synthetic construct
  source 1..73: mol_type = other DNA
gggccggtggcgaaatggcttaacgcaggaggcttttaacctcccatcgcgggttcgattcctgctcggccca

SEQ ID NO: 96 - DNA, 73 bp, synthetic construct
  source 1..73: mol_type = other DNA
agtccccgagtccgctggcgaaagacgctcggctttggaccgagagagcgaggttcgattcctcgggggacta

SEQ ID NO: 97 - DNA, 71 bp, synthetic construct
  source 1..71: mol_type = other DNA
gaggtcttggggtaatagaatcctactcgcctccagagcgagtgatgggagtgcaaatctctcagacctca

SEQ ID NO: 98 - DNA, 85 bp, synthetic construct
  source 1..85: mol_type = other DNA
gccggtggttgagcaatctggtcgctcacctgtctcgaaaacaggctcccattgtggaatgcaggttcgaatcctgccctcggcg

SEQ ID NO: 99 - DNA, 71 bp, synthetic construct
  source 1..71: mol_type = other DNA
acgaggatagtgtaatggaaacactccggtcagatggaccggcactacctgttcgattcaggttcctcgta

SEQ ID NO: 100 - DNA, 83 bp, synthetic construct
  source 1..83: mol_type = other DNA
gccgtggttgagtcaggcaaactcggttggattgctaatccaacggcctttatggtcagaaggttcgaatccttccctcggcg

SEQ ID NO: 101 - DNA, 73 bp, synthetic construct
  source 1..73: mol_type = other DNA
agtcggtttgtgtagcggccaatcatacgaggctctgaccctcgtgacagaggttcgaatcctctaccgacta

SEQ ID NO: 102 - DNA, 75 bp, synthetic construct
  source 1..75: mol_type = other DNA
gcgttcgtagccaaatcaggtaaggcgttccgctgataacggaaagatttgttggttcaaatccaaccggacgca

SEQ ID NO: 103 - DNA, 72 bp, synthetic construct
  source 1..72: mol_type = other DNA
gtcgggatgccagagtggtcacggcccggacagcagattcggtgtacgggggttcgaatccctctcccgact

SEQ ID NO: 104 - DNA, 74 bp, synthetic construct
  source 1..74: mol_type = other DNA
gcccacgtagcctaattggataaggcaatggccttcgaagccagagattgcaggttcgagtcctgtcgtgggcg

SEQ ID NO: 105 - DNA, 73 bp, synthetic construct
  source 1..73: mol_type = other DNA
aggcgtataaggtagtggactatcccgcgagtcttctaaacttgcgacaggggttcgaatccccttacgcctg

SEQ ID NO: 106 - DNA, 84 bp, synthetic construct
  source 1..84: mol_type = other DNA
gagtcggtacgagaaatggttaaactcacgaggcttaggacctcgcgctttagtgctttgcaggttcgaatcctgcccggctca

SEQ ID NO: 107 - DNA, 84 bp, synthetic construct
  source 1..84: mol_type = other DNA
gcatctgtagggaactggtgaaacccataggcttgaggggccttgcccgtaggggctttcttggttcgattccaagcgggtgca

SEQ ID NO: 108 - DNA, 83 bp, synthetic construct
  source 1..83: mol_type = other DNA
gagtggatagccaagtggtcaaaggcaggagagtcaaatcctcccggtatagtccttcgcaggttcgaatcctgctccgctca

SEQ ID NO: 109 - DNA, 85 bp, synthetic construct
  source 1..85: mol_type = other DNA
aggtcggtgcgccgagtctggtcaaaggcagggcacttaagatgccccgattagttcttcacaggttcaaatcctgtccggccta

SEQ ID NO: 110 - DNA, 71 bp, synthetic construct
  source 1..71: mol_type = other DNA
tggctggtggtccaatggtacgattcggcgtttgggacgccgagatatgggttcgactcccttccagccaa

SEQ ID NO: 111 - DNA, 75 bp, synthetic construct
  source 1..75: mol_type = other DNA
acgggtataattcaatctggtcagaatactccgcttacatcggagatgttgaaggttcaaatccttctgcccgta

SEQ ID NO: 112 - DNA, 75 bp, synthetic construct
  source 1..75: mol_type = other DNA
gggtgtgtggtccaactcggttaagataccgcattgacattgcggtgatttgaggttcaaatcctctcgcaccca

SEQ ID NO: 113 - DNA, 75 bp, synthetic construct
  source 1..75: mol_type = other DNA
agtcccctggggtagtttggccaatcctctgggcctttggagcccaggacgcgagttcaaatctcgcggggacaa

SEQ ID NO: 114 - DNA, 96 bp, synthetic construct
  source 1..96: mol_type = other DNA
ggcgcaaattggcaaccaacgctattttacgtcgttcagttccctcttttccgcctccggagccggaggtcccgggttcaaatcccggcgggtccg

SEQ ID NO: 115 - DNA, 64 bp, synthetic construct
  source 1..64: mol_type = other DNA
ggggtgtccccctaatgccggcctcccaagccggtgatcccgggttcaaatcccggcggccgca

SEQ ID NO: 116 - DNA, 80 bp, synthetic construct
  source 1..80: mol_type = other DNA
gcagtgggggttttagggggtgtcccccctaatgcgggcctttcgagcccgtgacccgggttcaaatcccggccgcggca

SEQ ID NO: 117 - DNA, 71 bp, synthetic construct
  source 1..71: mol_type = other DNA
gggggttgtgggggcgttacccccacagggctctggcccctgggaccagggttcgaatccctgcccggcta

SEQ ID NO: 118 - DNA, 105 bp, synthetic construct
  source 1..105: mol_type = other DNA
aggtggtcaggttttacattaaaaacgctaaaacgggggttgtgggggcgttccccccacagggctctggcccctgggaccagggttcgaatccctgcccggcta

SEQ ID NO: 119 - DNA, 64 bp, synthetic construct
  source 1..64: mol_type = other DNA
ggggtgcccccctaatgccggcctcccaagccggtgatcccgggttcaaatcccggcggccgca

SEQ ID NO: 120 - DNA, 71 bp, synthetic construct
  source 1..71: mol_type = other DNA
gggtgtgggggtaccccccaccatgggggcctgtcgagcccctgacccgggttcaaatcccggccgcggcg

SEQ ID NO: 121 - DNA, 3539 bp, synthetic construct
  source 1..3539: mol_type = other DNA
cggctaactaagcggcctgctgactttctcgccgatcaaaaggcattttgctattaagggattgacgagggcgatctgcgcagtaagatgcgccccgcattgcacctatagcccaactggtagaggcagcagacttgaaatctgctcagtgtgagttcgaatctctctaggtgtaccactttcttcccgagcgtgattgatggaattggcatacataccgtccttgaaagtcgggttttgagggttcgaatcccttgtcacgcaccaaattatgtaaaaaagcgccctaaaggcgcttttttactatctgcgatactcaaagattcgaacctgcagcaggtttgagttgagcgcagcgaaacaacggagccgctcgtgctggcgacggcccgaagggcgagcgaagcgagtcatcctgcacgacccaccaattcgaaaagcctgctcaacgagcaggcttttttgcatgcgggtccccaataattacgatttaaatttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagcctatgcctcgggcatccaagcagcaagcgcgttacgccgtgggcatagaatccaagcaaggagggaagatttatgtcacgctcctctaacgatgttacgcagcagggctcccgccctaaaacaaagtcgggtggctcttccatgggcatcattcgcacatgtcgttcaggccctgaccaagtcaaatccatgcgggctgcttcagattcattcggtcgtgagttcggagacgtagccacctactcccaacatcagccggactccgattactcggggaactcatcgcgttccaagacattcatcgcgtcggctgccttcgaccaagaagcggttgttggcgcttcagcggcttacgtttcacccaagtttgagcagccgcgttctgagatctatatctatgattcagcagtctccggagagcaccggcgtcagggcattgccaccgcgtcaatcaattcgtcaaagcatgaggccaacgcgtcaggtgcttatgtgatctacgtgcaagcagattacggtgacgatcccgcagtggcttcatatacaaagtcgggcatacgggaagaagtgatgcactttgatatcgacccatctaccgccacctaaagcgttggtaacagttacgataccagagaacactattgaggggagaccagaaacaaaaaaaggccgcgttagcggccttcaataattggttacttcacgagggattgcttttcattgagaagagaaaagaaaaccgccgatcctgtccaccgcattactgcaaggtagtggacaagaccggcggtcttaagttttttggctgaatttcgaatttggtacacctagagagattcgaactcacactgagcagatttcgagtctgctgcctctaccagttgggctataggtgcattaaatctttttagagaagtggtgcgtgacaagggattcgaaccctcaaaacccgactttcgaggacggtatgtatgccaattccatcaatcacgcaatagcgccggaaatgcccgatgggggtaatcattataaaaggaccttcttggagtgtcaacacgaatttctgtcgagagcgtcagtttcacctgttttacgtaaaaacccgcttcggcgggtttttacttttggaaggttgagcgaccactattcttcgtaaaagttctgactgctgattgtaattattagaaaaactcatccgacattgaatgaaactgtgatttattcatatcaggattatcaataccatatttttgaaatgaccgtttctgtgaagaaggagaaaactcacctgagcagttccatgagatggctgaatcctggtatcggtctgcgattccgactcgtccaacatcaatacaacctatcgatttcccctcgtcaaaaatcgagttatccgaagagaaatcaccatgagtgacgacggaatccggagagaatggtgacgatttatgcatttctttccagacttgttcaacaggccagccattacgctcgtcatcaaaatcagacgcatcaactgaaccgttattcattcgagattgcgcctgagctgaacgaaatacgcggtcagagttaaaaggacaattacaaacaggaatggaatgcgaccggcgtgagaacactgctgacgcatcaacaatattttcaccagaatcaggatattcttccgatacctggaatgctgttttcccggggatcgcagtggttgacgaccatgcatcatcaggagtacggataaaatgcttgatggtcggtgaaggcataaattccgtcgaccagtttgatcggaccatctcatctgtaacatcattggcaacggaacctttgccatgttttgaaaatgactctggcgcatcgggcttcccatatgatcgatagattgtcgcaccggattgcccgacattatcgcgagcccatttatacccatacgaatcagcatccatgttggaattcgatcgcggacgagagcaagacgtttcccgttgaatatgagacattaaacctccttaagtccgaatattattgaagcatttatcagggttattgtctcatgagcggatacatatttgaatgtatttagaaaaataaacaaataggggttccgcgaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggttaccaccgtcaaaaaaaacggcgctttttagcgccgtttttatttttcaaccttgcatgatcagtgca

SEQ ID NO: 122 - DNA, 3540 bp, synthetic construct
  source 1..3540: mol_type = other DNA
cggctaactaagcggcctgctgactttctcgccgatcaaaaggcattttgctattaagggattgacgagggcgtatctgcgcagtaagatgcgccccgcattgcacctatagcccaactggtagaggcagcagacttgaaatctactcagtgtgagttcgaatctctctaggtgtaccactttcttcccgagggggtgtaatcgaattggcataggtactggacttgaaattcaggttttgtgggttcgaatcccaccacccctaccaaattatgtaaaaaagcgccctaaaggcgcttttttactatctgcgatactcaaagattcgaacctgcagcaggtttgagttgagcgcagcgaaacaacggagccgctcgtgctggcgacggcccgaagggtgagcgaagcgagtcatcctgcacgacccaccaattcgaaaagcctgctcaacgagcaggcttttttgcatgcgggtccccaataattacgatttaaatttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagcctatgcctcgggcatccaagcagcaagcgcgttacgccgtgggcatagaatccaagcaaggagggaagatttatgtcacgctcctctaacgatgttacgcagcagggctcccgccctaaaacaaagtcgggtggctcttccatgggcatcattcgcacatgtcgttcaggccctgaccaagtcaaatccatgcgggctgcttcagattcattcggtcgtgagttcggagacgtagccacctactcccaacatcagccggactccgattactcggggaactcatcgcgttccaagacattcatcgcgtcggctgccttcgaccaagaagcggttgttggcgcttcagcggcttacgtttcacccaagtttgagcagccgcgttctgagatctatatctatgattcagcagtctccggagagcaccggcgtcagggcattgccaccgcgtcaatcaattcgtcaaagcatgaggccaacgcgtcaggtgcttatgtgatctacgtgcaagcagattacggtgacgatcccgcagtggcttcatatacaaagtcgggcatacgggaagaagtgatgcactttgatatcgacccatctaccgccacctaaagcgttggtaacagttacgataccagagaacactattgaggggagaccagaaacaaaaaaaggccgcgttagcggccttcaataattggttacttcacgagggattgcttttcattgagaagagaaaagaaaaccgccgatcctgtccaccgcattactgcaaggtagtggacaagaccggcggtcttaagttttttggctgaatttcgaatttggtacacctagagagattcgaactcacactgagcagatttcgagtctgctgcctctaccagttgggctataggtgcattaaatctttttagagaagtggtaggggtggtgggattcgaacccacaaaacctgaatttcgagtccagtacctatgccaattcgattacacccccaatagcgccggaaatgcccgatgggggtaatcattataaaaggaccttcttggagtgtcaacacgaatttctgtcgagagcgtcagtttcacctgttttacgtaaaaacccgcttcggcgggtttttacttttggaaggttgagcgaccactattcttcgtaaaagttctgactgctgattgtaattattagaaaaactcatccgacattgaatgaaactgtgatttattcatatcaggattatcaataccatatttttgaaatgaccgtttctgtgaagaaggagaaaactcacctgagcagttccatgagatggctgaatcctggtatcggtctgcgattccgactcgtccaacatcaatacaacctatcgatttcccctcgtcaaaaatcgagttatccgaagagaaatcaccatgagtgacgacggaatccggagagaatggtgacgatttatgcatttctttccagacttgttcaacaggccagccattacgctcgtcatcaaaatcagacgcatcaactgaaccgttattcattcgagattgcgcctgagctgaacgaaatacgcggtcagagttaaaaggacaattacaaacaggaatggaatgcgaccggcgtgagaacactgctgacgcatcaacaatattttcaccagaatcaggatattcttccgatacctggaatgctgttttcccggggatcgcagtggttgacgaccatgcatcatcaggagtacggataaaatgcttgatggtcggtgaaggcataaattccgtcgaccagtttgatcggaccatctcatctgtaacatcattggcaacggaacctttgccatgttttgaaaatgactctggcgcatcgggcttcccatatgatcgatagattgtcgcaccggattgcccgacattatcgcgagcccatttatacccatacgaatcagcatccatgttggaattcgatcgcggacgagagcaagacgtttcccgttgaatatgagacattaaacctccttaagtccgaatattattgaagcatttatcagggttattgtctcatgagcggatacatatttgaatgtatttagaaaaataaacaaataggggttccgcgaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctacggaaaaacgccagcaacgcggcctttttacggttcctggttaccaccgtcaaaaaaaacggcgctttttagcgccgtttttatttttcaaccttgcatgatcagtgca

SEQ ID NO: 123 - DNA, 1726 bp, synthetic construct
  source 1..1726: mol_type = other DNA
atgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcacctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtccgttgctcaatgtacctataaccagaccgttcagtcagatattacggccttttcaaagaccgtaaagaaaaataagcacaagttttatccggcctttattcacatttcggcccgctcgatgaatgctcatccggaatttcgtatggcaatgaaagacggtgagtcggtgatatgggattctgttcacccttgttacaccgttttccatgagcaaactgaaacgttttcctcttcatggtccgaataccacgacgatttccggcagttttcacacatatattcccaagatgtggcgtgttacggtgaaaactcagcctatttccctaaagggtttattgagaatatgtttttcgtctccgccaatccctgggtgtccttcacctcctttgattcaaacgtggccaatatggacaacttcttcgcccccgttttcaccatgggcaaatattatacgcaaggcgacaaggtgtcaatgccgtcagcgattcaggttcatcatgccgtttgtgatggcttccatgtcggccgcatgtcaaatgaatcgcaacagtactgcgatgagtggcagggcggggcgtaaaaggttgaaaaataaaaacggcgctaaaaagcgccgttttttttgacggtggtatatcagcgtaacaacggaaaccggccattgcgccggttttttttggcct

SEQ ID NO: 124 - DNA, 1851 bp, synthetic construct
  source 1..1851: mol_type = other DNA
atgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcaaggtggcacttttcggggaaatgtgcgcggaacccctatttgtttatttttctaaatacattcaaatatgtatccgctcatgagacaataaccctgataaatgcttcaataatattcaggttaccctacgatgtccattcaacatttccgtgtcgcctcgattcccttttttgcggcattttgctcgcctgtttttgctcacccagaaacgtcagtgaaagtaaaagatgctgaagatcagtcaggtgcacgagtgggttacatcgaatcagattcaaactctggtaagatctcggagtcctttcgccccgaagaacgttttccaatgatgtccacttttaaagtttcgtcatgtggcgcggtatcatcccgtattgacgccgggcaagagcaatcgggtcgccgcatacactattctcagaatgactcagttgagtactccccagtcacagaaaagcattcaacggatggcatgacagtacgcgaatcgtgctctgctgccataaccatgtctgataacactgcggccaactcatcgtcaacaacgatcggaggaccgaaggagtcaaccgctttttcacacaacatgggggatcatgtaactcgctcggatcgttgggaaccggagtcgaatgaagccataccaaacgacgagcgtgacaccacgatgcctgtagcaatggcaacaacgtcgcgcaaatcgtcaactggcgaatcgtcaacttcagcttcccggcaacaatcaatagactggatggaggcggataaagttgcaggaccatcgtcacgctccgcctcaccggctggctggtttattgctgataaatctggagccggtgagcgtgggtctcgcggtatcattgcagcatcagggccagatggtaagccctcccgtatcgtagttatctacacgacggggtcccaggcaactatggatgaacgaaatcgccagatcgctgagataggtgcctcttcgattaagcattggtaataatcagcgtaacaacggaaaccggccattgcgccggttttttttggcct

SEQ ID NO: 125 - DNA, 1690 bp, synthetic construct
  source 1..1690: mol_type = other DNA
atgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagggtccccaataattacgatttaaatttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagcctatgcctcgggcatccaagcagcaagcgcgttacgccgtgggcatagaatccaagcaaggagggaagatttatgtcacgctcctctaacgatgttacgcagcagggctcccgccctaaaacaaagtcgggtggctcttccatgggcatcattcgcacatgtcgttcaggccctgaccaagtcaaatccatgcgggctgcttcagattcattcggtcgtgagttcggagacgtagccacctactcccaacatcagccggactccgattactcggggaactcatcgcgttccaagacattcatcgcgtcggctgccttcgaccaagaagcggttgttggcgcttcagcggcttacgtttcacccaagtttgagcagccgcgttctgagatctatatctatgattcagcagtctccggagagcaccggcgtcagggcattgccaccgcgtcaatcaattcgtcaaagcatgaggccaacgcgtcaggtgcttatgtgatctacgtgcaagcagattacggtgacgatcccgcagtggcttcatatacaaagtcgggcatacgggaagaagtgatgcactttgatatcgacccatctaccgccacctaaaaggttgaaaaataaaaacggcgctaaaaagcgccgttttttttgacggtggtatatcagcgtaacaacggaaaccggccattgcgccggttttttttggcct

SEQ ID NO: 126 - DNA, 2693 bp, synthetic construct
  source 1..2693: mol_type = other DNA
ccaaaagtaaaaacccgccgaagcgggtttttacgtaaaacaggtgaaactgacttacgccccgccctgccactcatcgcagtactgttgcgattcatttgacatgcggccgacatggaagccatcacaaacggcatgatgaacctgaatcgctgacggcattgacaccttgtcgccttgcgtataatatttgcccatggtgaaaacgggggcgaagaagttgtccatattggccacgtttgaatcaaaggaggtgaaggacacccagggattggcggagacgaaaaacatattctcaataaaccctttagggaaataggctgagttttcaccgtaacacgccacatcttgggaatatatgtgtgaaaactgccggaaatcgtcgtggtattcggaccatgaagaggaaaacgtttcagtttgctcatggaaaacggtgtaacaagggtgaacagaatcccatatcaccgactcaccgtctttcattgccatacgaaattccggatgagcattcatcgagcgggccgaaatgtgaataaaggccggataaaacttgtgcttatttttctttacggtctttgaaaaggccgtaatatctgactgaacggtctggttataggtacattgagcaacggactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacaggtccatgggtatggacagttttccctttgatatgtaacggtgaacagttgttctacttttgtttgttagtcttgatgcttcactgatagatacaagagccataagaacctcagatacgtccgtatttagctgatatgttctccgatgtggttcgttgtttttgcgttgaccaggagaaggaaccatttgaatcatacttggattgcatgtcagacgaaaattttgcctcaaaagaggttgactgaatttttgcagttgaagcatcgtgcgatgttttacgtgatccgttacgtgagtaggaatcagatgtaatggttgttggtattttgtcaccattcatttttatctggttgttctctgattcggttactgaatccatttgtcgatccgattcaacttggaaaatcgacgtatcagtcgggcggcctcgcttatcaaccaccgatttcatattagagtaagtgttcgaatctttagatattggttttgaaacccattggtttgagcgtttaaactcatggtagttattttctgacatcgacatgaacttaaattcatctgaggaaatctctatatttgccttgtgagttttcttttgtgtcgattctttcgatgaccactcataaatccgcatagagtatttgttttcaaaagacttaacatgttctgaattatattttatgaattttttcgactggaatgaataaggtgatatctcttcagaaaaaaccgattccgatttttcagaggagaacttggcatagtttgtccactggaaaatctcaaagcctttaactgaaggattacggatttccacagtacgcgtcattgactcacgggttgctttagctgatacaccataagcattttcacgagagatgttcatcatctgagcgtattggttataagtgaaagataccgtccgttctttacgtgtagggttttcaatcgtggggttcgatgatgccacacagcacgaaatcgacttggtttcatgctccgttgagtcatagcgagaaatcgccgattcatttgctttgaaaacaaccgattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacg

SEQ ID NO: 127 - DNA, 2863 bp, synthetic construct
  source 1..2863: mol_type = other DNA
ttacgccccgccctgccactcatcgcagtactgttgcagttcattaagcatgcggccgacatggaagccatcacaaacggcatgatgaacctgaatcgccagcggcatcagcaccttgtcgccttgcgtataatatttgcccatggtgaaaacgggggcgaagaagttgtccatattggccacgttgagatcaaaggaggtgaaggacacccagggattggcggagacgaaaaacatattctcaataaaccctttagggaaataggccaggttttcaccgtaacacgccacatcttgggaatatatgtgtagaaactgccggaaatcgtcgtggtattcggaccagagagaggaaaacgtttcagtttgctcatggaaaacggtgtaacaagggtgaacagaatcccatatcaccagctcaccgtctttcattgccatacgaaattccggatgagcattcatcaggcgggcaagaatgtgaataaaggccggataaaacttgtgcttatttttctttacggtcttcagaaaggccgtaatatccagctgaacggtctggttataggtacattgagcaacggactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacaggtaccgcctttgagtgagctgataccgctcgccgcagtagaaaagatcaaaggatcttcttgagatcctttttttctgcgggggatcaggaccgctgccggagcgcaacccactcactacagcagagccatgtagggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggaggtttctcttctttcactttacggctagctcagccctaggtattatgctagcagccccatacgatataaggagggtgaatgaatcggacgtttgaccggaaggcataccgacaagaatcaatcgacgcggggttttccgccgaggatgccgaaaccatcgcatctcgcaccgtcatgcgtgcgccccgcgaaaccttccagtccgtcggctctatagtccagcaagctacggccaagatcgagcgcgactctgtgcaatcggctccccctgcctcgcccgcgccatctgccgccgtggagcgttctcgtcgttcggaacaggaggcggcaggttcggcgaagtctatgaccatcgatacacgaggtactatgacgaccaagaagcgaaaaaccgccggcgaggactcagcaaaacaggtctctgaagccaagcaggccgcgtcgtcaaaacacacgaagcagcagatcaaggaaatgcagtcatcctcattcgatattgcgccgtggccggacacgatgcgagcgatgccaaacgacacggcccgctctgcctcgttcaccacgcgcaacaagaaaatcccgcgcgaggcgtcacaaaacaaggtcattttccacgtcaacaaggacgtgaagatcacctacaccggcgtcgagtcacgggccgacgatgacgaatcggtgtggcagcaggtgtcagagtacgcgaagcgcacccctatcggcgagccgatcaccttcacgttctacgagtcatgccaggactcgggctggtccatcaatggccggtattacacgaaggccgaggaatgctcatctcgctcacaggcgacggcgatgggcttcacgtccgaccgcgttgggcactcagaatctgtgtcttcatcgcaccgcttccgcgtctcggaccgtggcaagaaaacgtcccgttgccaggtctcgatcgacgaggaaatcgtcgtgtcatttgctggcgaccactacacgaaattcatatgggagaagtaccgcaagtcatctccgacggcccgacggatgttcgactatttctcttcccaccgggagccgtacccgtcaaagtcagaaaccttccgctcaatgtgcggatctgattccacccgcgtgaagaagtggcgcgagcaggtcggcgaagcctgcgaagagtcacgaggctccggctcagtggaacacgcctgggtcaatgatgactcggtgcattgcaaacgctaatacctcgctctgctaatcctgttaccagtggctgctaccaccgtcaaaaaaaacggcgctttttagcgccgtttttatttttcaacctt

SEQ ID NO: 128 - DNA, 90 bp, Escherichia coli
  source 1..90: mol_type = genomic DNA
ggagagatgccggagcggctgaacggaccggtctcgaaaaccggagtaggggcaactctaccgggggttcaaatccccctctctccgcca

SEQ ID NO: 129 - DNA, 90 bp, synthetic construct
  source 1..90: mol_type = other DNA
ggaggcgtggcagagtggtttaatgcaccggtcttgaaaaccggcagtcgctccggcgactcataggttcaaatcctatcgcctccgcca

SEQ ID NO: 130 - DNA, 87 bp, synthetic construct
  source 1..87: mol_type = other DNA
ggaggcgtggcagagtggtttaatgcaccggtcttgaaaaccggcagtcgctccggcgactcataggttcaaatcctatcgcctccg

SEQ ID NO: 131 - DNA, 87 bp, synthetic construct
  source 1..87: mol_type = other DNA
ggaggcgtggcagagtggtttaatgcaccggtcttgaaaaccggcagtcgctccggcgactcataggttcgaatcctatcgcctccg

SEQ ID NO: 132 - DNA, 90 bp, synthetic construct
  source 1..90: mol_type = other DNA
ggaggtgtggcagagtggttgaatgcaccggtcttgaaaaccggcagtcgctccggcgattcataggttcgaatcctatcacctccgcca

SEQ ID NO: 133 - DNA, 92 bp, synthetic construct
  source 1..92: mol_type = other DNA
ggaaatgtggcagagcatggttgattgcgccgccctcgaaaagcggaggctgtgaaaacggcccgcaggttcgaatcctgccatttccgcca

SEQ ID NO: 134 - DNA, 87 bp, synthetic construct
  source 1..87: mol_type = other DNA
ggagagtagggagcatggtgctcaagcggtcttgaaaaccgtcccatagcgcaagcgatgatggttcgattccattactctccgcca

SEQ ID NO: 135 - DNA, 87 bp, synthetic construct
  source 1..87: mol_type = other DNA
ggagagtagggggcatggtgctcaagcggtcttgaaaaccgtcccattgcgcaagcgatgatggttcgattccattactctccgcca

SEQ ID NO: 136 - DNA, 87 bp, synthetic construct
  source 1..87: mol_type = other DNA
ggagagtagggagcatggtgctcaagcggtcttgaaaaccgtcccattgcgcaagcgatgatggttcgattccattactctccgcca

SEQ ID NO: 137 - DNA, 91 bp, synthetic construct
  source 1..91: mol_type = other DNA
ggagggtagggagcaatggtgctcaagcggtcttgaaaaccgtcccgttgaggatgactcgatgatggttcgattccattactctccgcca

SEQ ID NO: 138 - DNA, 90 bp, synthetic construct
  source 1..90: mol_type = other DNA
ggaaggtaggacatagtggtatgtaacaggtcttgaaaacctgcccgctgtagcgatatggtgatggttcgactccattaccttcctcca

SEQ ID NO: 139 - DNA, 90 bp, synthetic construct
  source 1..90: mol_type = other DNA
ggaagtacgggcgtagtggtatgcaactagtcttgaaaactagcccgctgtagcgatatggtgatggttcgactccatgtgcttccgcca

SEQ ID NO: 140 - DNA, 90 bp, synthetic construct
  source 1..90: mol_type = other DNA
agaagataggacgtagtggtacgtaaccggtcttgaaaaccggcccgctgtagtgatatggtgatggttcgactccattatcttctgcca

SEQ ID NO: 141 - DNA, 89 bp, synthetic construct
  source 1..89: mol_type = other DNA
ggaaggtggcgagcaacggcgctcaactagtcttgaaaactagggctgctcagagatgggtaagggttcgaatccttcactttccgcca

SEQ ID NO: 142 - DNA, 88 bp, synthetic construct
  source 1..88: mol_type = other DNA
ggtaggtagcggctaatggcagccaaacagtcttgaaaactgttgccactgtagagatacggtgagggttcgactcctttacttaccg

SEQ ID NO: 143 - DNA, 91 bp, synthetic construct
  source 1..91: mol_type = other DNA
tgagggtagggagcaatggtgctcaagcggtcttgaaaaccgtcccgttgaggatgactcgatgatggttcgattccattaccctcagcca

SEQ ID NO: 144 - DNA, 90 bp, synthetic construct
  source 1..90: mol_type = other DNA
ggaaggtagggcgtagtggtacgcaactagtcttgaaaactagcccgctgtagtgatacggtgatggttcgactccattaccttcctcca

SEQ ID NO: 145 - DNA, 88 bp, synthetic construct
  source 1..88: mol_type = other DNA
ggtaggtagcggctaatggtagccaaactgtcttgaaaacagttgccactgtagagatacggtgagggttcgactcctttacttaccg

SEQ ID NO: 146 - DNA, 88 bp, synthetic construct
  source 1..88: mol_type = other DNA
ggtaggtagcggctaatggtagccaaactgtcttgaaaacagttgccactgtagatatacggtgagggttcgactcctttacttaccg

SEQ ID NO: 147 - DNA, 90 bp, synthetic construct
  source 1..90: mol_type = other DNA
agaagataggacgtagtggtacgtaactggtcttgaaaaccagcccgctgtagtgatacggtgatggttcgactccattatcttctgcca

SEQ ID NO: 148 - DNA, 90 bp, synthetic construct
  source 1..90: mol_type = other DNA
ggaaggtagggcgcagcggtgcgcaaccaggtttgaaccctggcccattgtagcgatacggtgatggttcgactccattatcttcctcca

SEQ ID NO: 149 - DNA, 89 bp, synthetic construct
  source 1..89: mol_type = other DNA
ggaggctggggtgcatggtgcacaagctggtttgaacccagtcccgctgtagagatatggtgacagttcgattctgtcagcctcctcca

SEQ ID NO: 150 - DNA, 95 bp, synthetic construct
  source 1..95: mol_type = other DNA
ggtaggtcaatccaacggctggcgatggactccgacttgaaatcggttgatcgtagtgatacggtgtaagggttcgatccccttacctacctcca

SEQ ID NO: 151 - DNA, 95 bp, synthetic construct
  source 1..95: mol_type = other DNA
ggtaggtcaatccaacggctggcgctggactccgacttgaaatcggttgatcgtagtgatacggtgtaagggttcgatccccttacctaccccca

SEQ ID NO: 152 - DNA, 48 bp, synthetic construct
  source 1..48: mol_type = other DNA
gtccccggggccggggtcccagggtcaggtgtccctggggttggtaag

SEQ ID NO: 153 - protein, 16 aa, synthetic construct
VPGAGVPGSGVPGVGK

SEQ ID NO: 154 - protein, 13 aa, synthetic construct
LYGKPDAPELFLK

SEQ ID NO: 155 - DNA, 39 bp, synthetic construct
  source 1..39: mol_type = other DNA
tcatatgggaagcccgatgcgccagagtcattttcaaaa

SEQ ID NO: 156 - DNA, 30 bp, synthetic construct
  source 1..30: mol_type = other DNA
tcagaaaacggaacttattacgtccatcgt

SEQ ID NO: 157 - protein, 10 aa, synthetic construct
LENGTYYVHR

(19) United States
(12) Patent Application Publication
(10) Pub. No.: US 2026/0258406 A1
(43) Pub. Date: Sep. 3, 2026

(54) METHODS AND COMPOSITIONS FOR CONFERRING CELLULAR RESISTANCE TO VIRAL INFECTION

(71) Applicant: President and Fellows of Harvard College, Cambridge, MA (US)

(72) Inventors: Akos J. Nyerges, Cambridge, MA (US); George M. Church, Cambridge, MA (US); Svenja Vinke, Cambridge, MA (US)

(73) Assignee: President and Fellows of Harvard College, Cambridge, MA (US)

(21) Appl. No.: 18/877,716

(22) PCT Filed: Jun. 30, 2023
(86) PCT No.: PCT/US2023/069468

§ 371 Date: Dec. 20, 2024

Related U.S. Application Data
(60) Provisional application No. 63/358,474, filed on Jul. 5, 2022.

(51) Int. Cl. C12N 15/11; C12N 15/86

(52) U.S. Cl. CPC C12N 15/11; C12N 15/86; C12N 2795/10121; C12N 2840/60

(57) ABSTRACT

Provided herein are methods and compositions for conferring cellular resistance to viral infection.

### CROSS-REFERENCE TO RELATED APPLICATIONS

This application claims the benefit under 35 U.S.C. § 119(e) of U.S. Provisional Application No. 63/358,474, filed Jul. 5, 2022, entitled “METHODS AND COMPOSITIONS FOR CONFERRING CELLULAR RESISTANCE TO VIRAL INFECTION,” the entire disclosure of which is hereby incorporated by reference in its entirety.

### GOVERNMENT LICENSE RIGHTS

This invention was made with government support under 2123243 awarded by National Science Foundation (NSF) and under DE-FG02-02ER63445 awarded by U.S. Department of Energy (DOE). The government has certain rights in the invention.

### REFERENCE TO AN ELECTRONIC SEQUENCE LISTING

The contents of the electronic sequence listing (H049870768WO00-SEQ-KVC.xml; Size: 165,708 bytes; and Date of Creation: Jun. 29, 2023) is herein incorporated by reference in its entirety.

### BACKGROUND

Removing cellular transfer RNAs (tRNAs), making their cognate codons unreadable, creates a genetic firewall that prevents viral replication and horizontal gene transfer. However, numerous viruses and mobile genetic elements encode parts of the translational apparatus, including tRNAs, potentially rendering a genetic-code-based firewall ineffective.

### SUMMARY

The data provided herein shows that such horizontally transferred tRNA genes can enable viral replication in Escherichia coli cells despite the genome-wide lack of three codons and the previously essential cognate tRNAs and release factor 1. By repurposing viral tRNAs, recoded cells were developed bearing an amino-acid-swapped genetic code that reassigns two of the six serine codons to leucine during translation. This amino acid-swapped genetic code renders cells completely resistant to viral infections by mistranslating viral proteomes and prevents the escape of synthetic genetic information by engineered reliance on serine codons to produce leucine-requiring proteins. The third free codon was also repurposed to biocontain this virus-resistant host via dependence on an amino acid not found in nature.

Some aspects provide a method of conferring viral resistance to a cell, comprising delivering to the cell an engineered nucleic acid encoding the first viral tRNA variant, wherein the first viral tRNA variant binds to a first messenger ribonucleic acid (mRNA) codon and is charged with an amino acid not encoded by the first mRNA codon.

Other aspects provide a method of conferring viral resistance to a cell, comprising: delivering to the cell a first viral transfer ribonucleic acid (tRNA) variant, wherein the first viral tRNA variant binds to a first messenger ribonucleic acid (mRNA) codon and is charged with an amino acid not encoded by the first mRNA codon.

Some aspects provide cell, comprising: an engineered nucleic acid encoding the first viral tRNA variant, wherein the first viral tRNA variant binds to a first messenger ribonucleic acid (mRNA) codon and is charged with an amino acid not encoded by the first mRNA codon, wherein the cell is resistant to viral infection.

Other aspects provide a cell, comprising: a first viral transfer ribonucleic acid (tRNA) variant, wherein the first viral tRNA variant binds to a first messenger ribonucleic acid (mRNA) codon and is charged with an amino acid not encoded by the first mRNA codon, wherein the cell is resistant to viral infection.

In some embodiments, the cell comprises second viral tRNA variant, wherein the second viral tRNA variant binds to a second mRNA codon and is charged with an amino acid not encoded by the second mRNA codon, and wherein the second mRNA codon is different from the first mRNA codon.

In some embodiments, the cell comprises an engineered nucleic acid encoding the second viral tRNA variant.

In some embodiments, the cell comprises a second viral tRNA variant, wherein the second viral tRNA variant binds to a second mRNA codon and is charged with an amino acid not encoded by the second mRNA codon, and wherein the second mRNA codon is different from the first mRNA codon.

In some embodiments, the method further comprises delivering to the cell the second viral tRNA variant or an engineered nucleic acid encoding the second viral tRNA variant.

In some embodiments, the first mRNA codon encodes an amino acid selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

In some embodiments, the first mRNA codon encodes a serine, optionally wherein the first mRNA codon is selected from UCA, UCG, UCC, and UCU, preferably UCA and UCG.

In some embodiments, the second mRNA codon encodes an amino acid selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

In some embodiments, the second mRNA codon encodes a serine, optionally wherein the second mRNA codon is selected from UCA, UCG, UCC, and UCU, preferably UCA and UCG.

In some embodiments, the first amino acid is selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine, optionally wherein the first amino acid is leucine.

In some embodiments, the second amino acid is selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine, optionally wherein the second amino acid is leucine.

In some embodiments, the cell does not comprise an endogenous tRNA that binds to the first mRNA codon.

In some embodiments, the cell does not comprise an endogenous tRNA that binds to the second mRNA codon.

In some embodiments, the cell expresses an essential gene encoding a protein that comprises a nonstandard amino acid.

In some embodiments, the cell comprises a third viral tRNA variant, wherein the third viral tRNA variant binds to a TAG stop codon and is charged with the nonstandard amino acid.

In some embodiments, the method further comprises delivering to the cell an engineered nucleic acid encoding the third viral tRNA variant.

In some embodiments, the nonstandard amino acid is L-4,4′-biphenylalanine (bipA).

In some embodiments, the cell does not comprise an endogenous tRNA that binds to a TAG stop codon.

In some embodiments, the cell is a mammalian cell, a yeast cell, a plant cell, or a bacterial cell.

In some embodiments, the cell further comprises an mRNA comprising the first mRNA codon to which the first viral tRNA variant binds. In some embodiments, the mRNA further comprises the second mRNA codon to which the second viral tRNA variant binds.

In some embodiments, the cell comprises an artificial genetic code in which an mRNA codon encodes an amino acid (e.g., leucine) different from its natural amino acid identity (e.g., serine).

In some embodiments, the cell comprises a vector (e.g., plasmid) comprising a gene of interest, wherein the vector can only be expressed in the cell comprising the artificial genetic code (i.e., cannot be expressed in a cell comprising a canonical genetic code). In some embodiments, the method further comprising delivering to the cell the vector (e.g., plasmid) comprising a gene of interest, wherein the vector can only be expressed in the cell comprising the artificial genetic code. In some embodiments, the vector is a plasmid comprising a sequence selected from Table 4 or a sequence having at least 80%, at least 85%, at least 90%, or at least 95% identity to a sequence selected from Table 4.

In some embodiments, the first and/or second viral tRNA variant comprises the sequence of any one of SEQ ID NOs: 1-6. In some embodiments, the first and/or second viral tRNA variant is selected from the tRNA variants of Table 3.

### BRIEF DESCRIPTION OF DRAWINGS

FIGS. 1A-1D show the discovery of mobile TCR codon suppressor tRNAs in E. coli Syn61Δ3. (FIG. 1A) We screened the mobile tRNAome for tRNAs that can simultaneously suppress TCA and TCG (i.e., TCR) codons by computationally identifying tRNA genes in mobile genetic elements (1.) and then synthesizing select candidates as an oligonucleotide library and cloning these variants under the control of a constitutive bacterial promoter into a plasmid vector carrying an nptII_{40TCA,68TCG,104TCA,251TCG} marker (conferring kanamycin resistance) (2.). Following the transformation of this library into Syn61Δ3 (3.), in which the deletion of serU (encoding tRNA-Ser_{CGA}) and serT (encoding tRNA-Ser_{UGA}) makes TCG and TCA codons unreadable, only variants carrying functional TCR suppressor tRNAs survive kanamycin selection (4.). Finally, PCR amplification and high-throughput sequencing of the tRNA inserts from kanamycin-resistant clones identified suppressor tRNAs (5.). (FIG. 1B) Multiple sequence alignment of mobile TCR codon suppressor tRNAs. The anticodon region is indicated by an asterisk, while the host's serine-tRNA-ligase identity elements are represented by a star. (FIG. 1C) Viral TCR suppressor tRNAs decode TCA codons as serine. (FIG. 1D) The expression of viral TCR suppressor tRNAs and serU (tRNA-Ser_{CGA}) restores the replication of T6 bacteriophage in Syn61Δ3. Cultures were infected at an MOI of 0.01 with T6 phage, and the figure shows total T6 titer after 24 hours of incubation. All experiments were performed in three independent replicates, the dots represent data from independent replicates, and the bar graph represents the mean. ND represents below the detection limit (i.e., <10³ PFU/ml); ns indicates lack of significance (p>0.5) based on Student's t test.

FIGS. 2A-2E show the lytic phages of Syn61Δ3. (FIG. 2A) Titer of lytic Syn61Δ3 phage isolates. Early exponential phase cultures of Syn61Δ3 were infected at an MOI of 0.001 with corresponding phages and free phage titers were determined after 24 hours of incubation. All experiments were performed in two independent replicates; dots represent data from independent replicates; bar graphs represent the mean; the error bars represent SEM. (FIG. 2B) Single-step growth curve of S13_4 lytic Syn61Δ3 phage isolate. Growth experiment was performed in triplicate, and dots represent total viral titer. (FIG. 2C) Genomic maps of tRNA operons in phage isolates. Black arrows represent predicted tRNA genes; circled arrows denote tRNA genes identified in our earlier TCR codon suppressor screen (FIG. 1B); gray arrow with a star represents homing endonuclease gene, while gray arrows represent protein-coding genes. 1.=REP1; 2.=REP2; 3.=REP4; 4.=REP6; 5.=REP12. (FIG. 2D) Viral tRNA operon-expressed tRNAs translate TCR codons. Syn61Δ3 expressing elastin16 GCA(alanine)-sfGFP-His6 served as wild-type expression control and the elastin16TCR-sfGFP-His6 expression was compared with and without the coexpression of the REP12 viral tRNA operon. xxx marks the analyzed codon, TCA or GCA. A.U. denotes arbitrary fluorescence units. Error bars represent SD. (FIG. 2E) Viral tRNA operon-expressed tRNAs decode TCR codons as serine. The amino acid identity of the translated TCA codon within elastin16TCA-sfGFP-His6 was confirmed by tandem mass spectrometry from Syn61Δ3 cells containing the REP12 tRNA operon and its cognate promoter. The figure shows the amino acid sequence and MS/MS spectrum of the analyzed elastin16 TCR peptide. MS/MS data was collected once.

FIGS. 3A-3E show how amino-acid-swapped genetic code provides multi-virus resistance. (FIG. 3A) The creation of an E. coli GRO, Ec_Syn61Δ3-SL, in which both TCA and TCG-naturally serine-meaning-codons are translated as leucine. The introduction of bacteriophage-derived Leu-tRNA_{UGA} and Leu-tRNA_{CGA} to Syn61Δ3 reassigns TCA and TCG codons to leucine, while the reassignment of the TAG stop codon to encode L-4,4′-biphenylalanine (bipA) in essential genes of the host ensures the biocontainment of Ec_Syn61Δ3-SL. (FIG. 3B) The reassignment of TCR codons as leucine within the coding sequence of aph3Ia_{29×Leu→TCR} in Syn61Δ3-LS was confirmed by tandem mass spectrometry. (FIG. 3C) Schematic of viral infection in Ec_Syn61Δ3-SL. The reassignment of sense codons TCA and TCG to leucine in Ec_Syn61Δ3-SL provides multivirus resistance by mistranslating the viral proteome. (FIG. 3D) Bacteriophage-derived Leu-tRNA_{UGA} and Leu-tRNA_{CGA} expression in Syn61Δ3 provides multivirus resistance. The figure shows the titer of lytic Syn61Δ3 phages following the infection of the corresponding Leu-tRNA_{YGA}-expressing Syn61Δ3 strain with a mixture of twelve distinct lytic Syn61Δ3 phage. Early exponential phase cultures of Syn61Δ3 carrying the corresponding Leu-tRNA_{Y}GA expression construct were infected at an MOI of 0.1 with corresponding phage mixture, free phages were removed, and phage titers were determined after 24 hours of incubation (Methods). All experiments were performed in three independent replicates; dots represent data from independent replicates; bar graphs represent the mean; the error bars represent SD. (FIG. 3E) Mistranslated viral protein synthesis in Ec_Syn61Δ3-SL. The figure shows the amino acid sequence and MS/MS spectrum of a bacteriophage-expressed protein, together with its viral genomic sequence, in which the naturally serine-coding TCA codon is mistranslated as leucine. The experiment was performed by infecting Ec_Syn61Δ3-SL cells, expressing Leu9-tRNA_{YGA} from Escherichia phage OSYSP, with the REP12 phage at an MOI=12, and the proteome of infected cells was analyzed by tandem mass spectrometry (Methods).

FIGS. 4A-4C show how the addiction of synthetic genetic information to a genetic code in which TCR codons encode leucine prevents horizontal gene transfer. (FIG. 4A) We developed a set of plasmid vectors, termed the pLS plasmids, that rely on TCR codons to express leucine containing proteins. pLS plasmids can only function in Ec_Syn61Δ3-SL expressing bacteriophage-derived synthetic tRNA-Leu_{Y}GA tRNAs, and encoded proteins on pLS plasmids become mistranslated in cells bearing the canonical genetic code. (FIG. 4B) The pLS plasmids offer multiple mutually orthogonal antibiotic resistance markers together with low to high copy-number origins-of-replication that are addicted to a genetic code in which leucine is encoded as TCR codons. Number in parenthesis marks the number of Leu_{TCR} codons. (FIG. 4C) The transformation of pLS plasmids into wild-type E. coli K-12 MG1655 cells results in no escapees after 168 hours (7 days) of incubation. In each case, 1 g pLS plasmid from Ec_Syn61Δ3-SL cells was electroporated into E. coli K-12 MG1655 cells (electroporation efficiency=~3×10⁹ transformants/g, based on transformation with a plasmid carrying a pUC origin-of-replication and kanamycin resistance (pUC-Kan^{R})) and then plated on agar plates containing the corresponding antibiotic. Experiments were performed in triplicates; data points represent data from independent experiments.

### DETAILED DESCRIPTION

The universal genetic code allows organisms to exchange functions through horizontal gene transfer (HGT) and enables recombinant gene expression in heterologous hosts. However, the shared language of the same code permits the undesired spread of antibiotic resistance genes and allows viruses to replicate, to kill both pro- and eukaryotic cells, and to cause diseases. Horizontal gene transfer also threatens the safe use of Genetically Modified Organisms (GMOs) by enabling the release and spread of their engineered genetic information into natural ecosystems. It is widely hypothesized that Genomically Recoded Organisms (GROs), whose genomes have been systematically redesigned to confer an alternate genetic code, would offer genetic isolation from natural ecosystems by obstructing the translation of horizontally transferred genetic material, including both resistance to viral infections and horizontal gene transfer. Indeed, the genome-wide removal of TAG stop codons and release factor 1 (RF1) from Escherichia coli, which abolishes cells' ability to terminate translation at TAG stop codons, provides substantial resistance to bacteriophages and horizontal plasmid transfer. Most recently, a strain of E. coli, Syn61Δ3, was created with a synthetic recoded genome in which all annotated instances of two serine codons, TCG and TCA (together TCR), and the TGA stop codon were replaced with synonymous alternatives, and the corresponding serine tRNA genes (serU and serT) and RF1 (prfA) have been deleted. Syn61Δ3 resists a broad range of phages without detectable viral replication, including those that could overcome RF1-deletion-based resistance.

However, numerous viruses and mobile genetic elements encode parts of the translational apparatus, ranging from single transfer RNA (tRNA) genes and release factors up to lacking only ribosomal genes for a fully host-independent translation. These translational elements allow viruses to reduce their dependency on host translational processes by substituting elements of the translational apparatus or, in more extreme cases, even alter the host's genetic code during viral replication. Similarly, mobile genetic elements that encode transfer RNAs are widespread in nature. Recent studies highlighted the presence of mobile tRNA genes in diverse species, ranging from plasmids to actively spreading conjugative elements capable of decoding all twenty amino acids with their encoded tRNAs. Therefore, the selection pressure posed by the altered genetic codes of GROs might facilitate the rapid evolution of viruses and mobile genetic elements capable of crossing a genetic-code-based barrier.

Herein, the data shows that horizontally transferred tRNA genes can readily substitute cellular tRNAs in Genomically Recoded Organisms and thus abolish genetic-code-based resistance to viral infections and HGT. Next, by repurposing virus-encoded tRNAs, an amino-acid-swapped genetic code was develop that—by reassigning the amino acid identity of two sense codons provides complete virus resistance and enables the tight biocontainment of engineered genetic information. These developments provide a fundamental advance toward engineering multi-virus-resistant cell lines and the safer use of GMOs in open environments.

### Cellular Resistance to Horizontal Gene Transfer and Viral Infection

Horizontal gene transfer (HGT) (also known as lateral gene transfer (LGT)) is the movement of genetic material between organisms other than by the (vertical) transmission of DNA from parent to offspring (reproduction). HGT is the primary mechanism for the spread of antibiotic resistance in bacteria, and plays an important role in the evolution of bacteria that can degrade novel compounds such as human-created pesticides and in the evolution, maintenance, and transmission of virulence. It often involves temperate bacteriophages and plasmids. Genes responsible for antibiotic resistance in one species of bacteria, for example, can be transferred to another species of bacteria through various mechanisms of HGT such as transformation, transduction and conjugation, subsequently arming the antibiotic resistant genes' recipient against antibiotics. The rapid spread of antibiotic resistance genes in this manner is becoming medically challenging to deal with. Ecological factors may also play a role in the HGT of antibiotic resistant genes. It is also postulated that HGT promotes the maintenance of a universal life biochemistry and, subsequently, the universality of the genetic code.

Some aspects of the present disclosure provide methods of preventing organisms from exchange functions through HGT and thus preventing, for example, recombinant gene expression in heterologous hosts. This may, for example, prohibit the undesired spread of antibiotic resistance genes and prevent viruses from replicating, to kill both pro- and eukaryotic cells, ultimately preventing diseases. The methods provided herein may also prevent the release and spread of engineered genetic information from genetically modified organisms into natural ecosystems. For example, the methods provided herein would offer genetic isolation from natural ecosystems by obstructing the translation of horizontally transferred genetic material, including both resistance to viral infections and horizontal gene transfer.

Cellular “resistance” to viral infection refers to a decrease (relative to a naturally occurring environment) in cellular susceptibility to viral infection, for example, by arming a cell with the molecular machinery (e.g., viral tRNA variant) to prohibit viral replication.

### Engineered Host Cells

Cells according to the present disclosure include any cell into which an engineered nucleic acid encoding a viral tRNA variant can be introduced and expressed as described herein. The cells are considered “engineered” cells because they include an engineered nucleic acid, i.e., they do not exist in nature with that particular engineered nucleic acid. It should be understood that the basic concepts of the present disclosure are not limited by cell type. Cells provided by the present disclosure include prokaryotic and eukaryotic cells. Prokaryotic cells include bacterial cells (e.g., Escherichia coli) and archaeal cells. Eukaryotic cells include animal cells, plant cells, fungi cells, protist cells, and yeast cells. In some embodiments, the eukaryotic cells are mammalian cells. In some embodiments, the mammalian cells are human cells (e.g., HEK293 or K562 cells), primary cells, or pluripotent cells. Primary cells are cells taken directly from living tissue. Pluripotent cells are cells that are able to develop into many different types of cells or tissues. In some embodiments, the pluripotent cells are stem cells, such as human induced pluripotent stem cells (iPSCs).

### Amino Acid Swapping

In cells, messenger mRNA codons (3-nucleotide sequences) encode (code for) one of twenty individual amino acids: alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, or valine. One (e.g., a first) mRNA codon is considered to be different from another (e.g., a second) mRNA codon if the former mRNA codon encodes an amino acid that not the same as the amino acid encoded by the latter. Amino acid swapping herein refers to the process in a cell by which a viral tRNA variant inserts an amino acid in a growing polypeptide chain in response to (following binding to) an mRNA codon encoding an amino acid that is different from the amino acid inserted by the tRNA variant. For example, amino acid has occurring in a cell when in response to a serine mRNA codon, such as UCA or UCG, the tRNA instead is charged with and inserts a leucine residue in a growing polypeptide chain.

Table 1 provides the mRNA codon sequences for the standard genetic code.

TABLE 1
Standard Genetic Code

| | | 2^{nd} base in codon | | | | | |
|---|---|---|---|---|---|---|---|
| | | U | C | A | G | | |
| 1st | U | Phe | Ser | Tyr | Cys | U | 3rd |
| base in | | Phe | Ser | Tyr | Cys | C | base in |
| codon | | Leu | Ser | STOP | STOP | A | codon |
| | | Leu | Ser | STOP | Trp | G | |
| | C | Leu | Pro | His | Arg | U | |
| | | Leu | Pro | His | Arg | C | |
| | | Leu | Pro | Gin | Arg | A | |
| | | Leu | Pro | Gin | Arg | G | |
| | A | Ile | Thr | Asn | Ser | U | |
| | | Ile | Thr | Asn | Ser | C | |
| | | Ile | Thr | Lys | Arg | A | |
| | | Met | Thr | Lys | Arg | G | |
| | G | Val | Ala | Asp | Gly | U | |
| | | Val | Ala | Asp | Gly | C | |
| | | Val | Ala | Glu | Gly | A | |
| | | Val | Ala | Glu | Gly | G | |

A “nonstandard amino acid” refers to an amino acid that has been chemically modified. Non-limiting examples of nonstandard amino acids include L-4,4′-biphenylalanine (bipA), cystine, desmosine, and isodesmosine, hydroxyproline and hydroxylysine, gamma-carboxyglutamate, phosphoserine, phosphothreonine, and phosphotyrosine, N-acetyl lysine, methyllysine, and inositol.

### Viral Transfer RNA Variants

Transfer RNA (tRNA) is a small RNA molecule that serves as a physical link (or adaptor) between a messenger RNA (mRNA) and a growing chain of amino acids that make up a polypeptide (e.g., protein). tRNAs are a necessary component of translation. Each time an amino acid is added to the chain, a specific tRNA pairs with (binds to) its complementary 3-nucleotide sequence on the mRNA (to a specific mRNA codon), ensuring that an amino acid (e.g., under “normal” circumstances, ensuring that the appropriate amino acid encoded by the codon to which the tRNA binds) is inserted into the polypeptide being synthesized. A tRNA “variant” is a tRNA that performs an amino acid swapping function in the cell, i.e., inserts an amino acid in a growing polypeptide chain in response to an mRNA codon encoding an amino acid that is different from the one inserted by the tRNA variant. A tRNA “binds” to an mRNA codon through canonical Watson-Crick base pairing (i.e., A-U base pairing, G-C base pairing). In some embodiments, a tRNA variant binds to an mRNA codon that encodes an amino acid that is different from the amino acid that is bound to the tRNA variant. In some embodiments, a tRNA variant is bound to or “charged” with a leucine amino acid but binds to an mRNA codon that encodes a serine amino acid under normal conditions.

A tRNA is “charged” with an amino acid through a process referred to as aminoacylation—the process of adding an aminoacyl group to a compound. Aminoacylation covalently links an amino acid to the CCA 3′ end of a tRNA molecule. Each tRNA is aminoacylated (or “charged”) with a specific amino acid by an aminoacyl tRNA synthetase. There is normally a single aminoacyl tRNA synthetase for each amino acid, despite the fact that there can be more than one tRNA, and more than one anticodon for an amino acid. Recognition of the appropriate tRNA by the synthetases is not mediated solely by the anticodon, and the acceptor stem often plays a prominent role.

In some embodiments, tRNA variants that are charged with an amino acid and bind to an mRNA codon that encodes a different amino acid are identified by generation and screening of mutagenized tRNA libraries. In some embodiments, tRNA variants that are charged with an amino acid and bind to an mRNA codon that encodes a different amino acid are identified by generation and screening of libraries of naturally-occurring viral tRNA libraries. In some embodiments, a gene encoding a tRNA is mutagenized. In some embodiments, a gene encoding a tRNA is naturally-occurring. In some embodiments, a plurality of genes encoding tRNA variants are used to produce a tRNA variant library. In some embodiments, a gene encoding a tRNA variant is incorporated into a plasmid (expression construct) under the control of a promoter. In some embodiments, a gene encoding a tRNA variant is inserted (transformed or electroporated) into a cell. In some embodiments, cells comprising tRNA variants that are charged with an amino acid and that bind to an mRNA codon that encodes a different amino acid are identified and selected. In some embodiments, the cells require a protein that is dependent on leucine. In some embodiments, the leucine codons in the protein are swapped to serine codons. In some embodiments, only cells that comprise a tRNA variant that is charged with a leucine but binds to a serine codon can produce the protein. In some embodiments, the cells comprise a selection system. In some embodiments, the selection system is an antibiotic selection system. In some embodiments, the cells are exposed to kanamycin. In some embodiments, only cells that are kanamycin resistant are selected. In some embodiments, kanamycin resistance depends on the presence of a tRNA variant that is charged with a leucine and that binds to an mRNA codon that encodes a serine.

In some embodiments, the tRNA variant is derived from a virus. In some embodiments, the virus is a lytic virus. In some embodiments, the virus is a lysogenic virus. In some embodiments, the virus infects prokaryotes. In some embodiments, the virus infects bacteria. In some embodiments, the virus is a bacteriophage (phage). In some embodiments, the virus infects bacteria of the family Enterobacteriaceae. In some embodiments, the virus infects bacteria of the genus Escherichia. In some embodiments, the virus infects Escherichia coli. In some embodiments, the virus infects bacteria of the genus Bacillus. In some embodiments, the virus infects bacteria of the genus Lactobacillus. In some embodiments, the virus is derived from an environmental source. In some embodiments, the virus is a phage of the Caudovirales order. In some embodiments, the virus is a phage of the Caudovirales order and the Myoviridae family.

In some embodiments, the virus infects eukaryotes. In some embodiments, the virus infects fungal cells. In some embodiments, the virus infects yeast. In some embodiments, the virus infects Saccharomyces cerevisiae. In some embodiments, the virus infects Yarrowia lipolytica. In some embodiments, the virus infects industrial microbes. In some embodiments, the virus infects animal cells. In some embodiments, the virus infects mammalian cells. In some embodiments, the virus infects rodent cells. In some embodiments, the virus infects human cells. In some embodiments, the virus infects archaeal cells.

### Engineered Nucleic Acids

Also provided herein are engineered nucleic acids, for example, encoding a viral tRNA variant. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) amino occurring nucleic acid molecules. Recombinant and synthetic nucleic acids also include those molecules that result from the replication of either of the foregoing. Engineered nucleic acids of the present disclosure may be produced using standard molecular biology methods (see, e.g., Green and Sambrook, Molecular Cloning, A Laboratory Manual, 2012, Cold Spring Harbor Press), or by any method known in the art.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine. In some aspects, the set of nucleic acids comprises a sequence encoding a fusion protein as described herein. In some embodiments, the set of nucleic acids comprises an open reading frame encoding a fusion protein as described herein. In some embodiments, the set of nucleic acids comprises one or more nucleic acids further comprising a sequence encoding an inducible promoter operably linked to the sequence encoding the fusion protein. In some embodiments, the set of nucleic acids comprises nucleic acids further comprising a sequence encoding an inducible promoter operably linked to the sequence encoding the fusion protein.

A promoter is a DNA sequence recognized by the synthetic machinery of the cell, or introduced synthetic machinery, required to initiate the specific transcription of a gene. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control transcriptional initiation and/or expression of that sequence.

An inducible promoter is one that is characterized by initiating or enhancing transcriptional activity when in the presence of, influenced by or contacted by an inducing agent. An inducing agent may be endogenous or a normally exogenous condition, compound or protein that contacts an engineered nucleic acid in such a way as to be active in inducing transcriptional activity from the inducible promoter. Inducible promoters for use in accordance with the present disclosure include any inducible promoter described herein or known to one of ordinary skill in the art. In some embodiments provided herein, the inducible promoter is a doxycycline-inducible promoter.

The present disclosure provides, in some embodiments, a vector comprising a promoter operably linked to the open reading frame of a nucleic acid encoding a fusion protein as described herein. The term “vector,” as used herein, refers to a nucleic acid that is able to enter into a host cell, mutate and replicate within the host cell, and then transfer a replicated form of the vector into another host cell. As presented herein, vectors include plasmids, viral vectors, cosmids, and artificial chromosomes. Vectors also include any template formats known in the art (e.g., linear DNA generated by polymerase chain reaction (PCR), or chemical synthesis).

### EXAMPLES

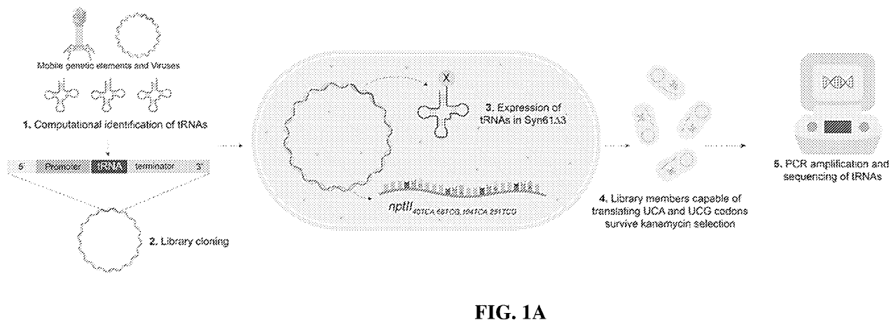

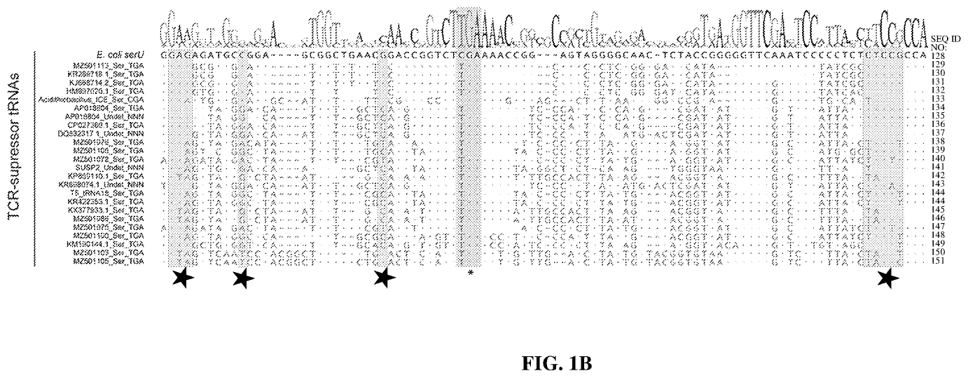

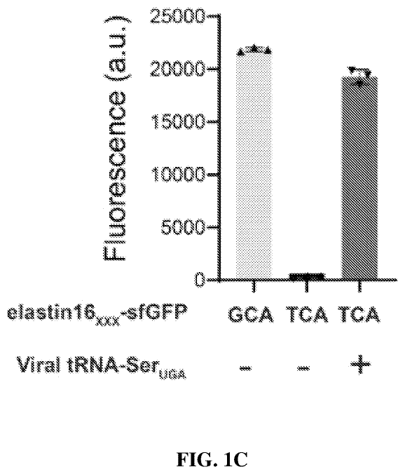

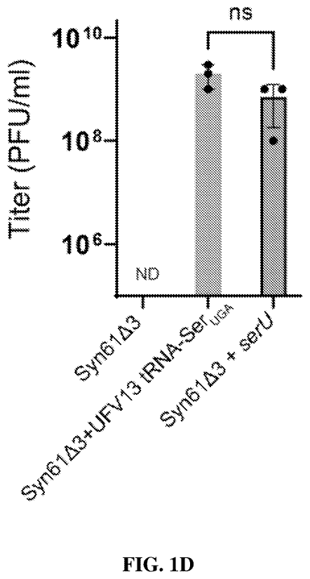

The data provided below demonstrates that tRNAs expressed by horizontally transferred genetic elements, including bacteriophages, plasmids, and integrative conjugative elements—the mobile tRNAome-readily substitute cellular tRNAs and can abolish the genetic-code-based isolation of Genomically Recoded Organisms (GRO). By screening more than a thousand mobile tRNAome-derived tRNAs, we discovered tRNA species capable of restoring viral replication in a recently developed E. coli GRO, Syn61Δ3, lacking sense TCR serine codons and the TAG stop codon together with their cognate serine tRNAs and release factor 1. We have also shown that the mobile tRNAome is not limited to bacteria, as multiple Archaeal and Eukaryotic viruses also carry predicted tRNA genes. We hypothesize that in future studies, our general, multiplexed tRNA suppressor screen described herein (FIG. 1A) will facilitate the analysis of mobile tRNA genes in these organisms.

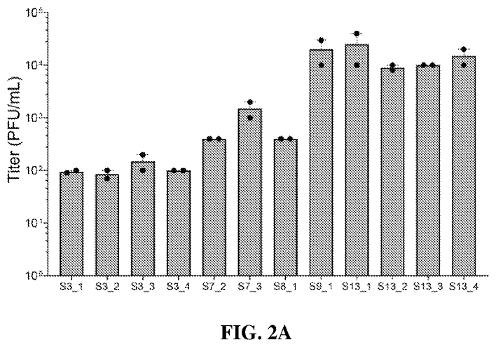

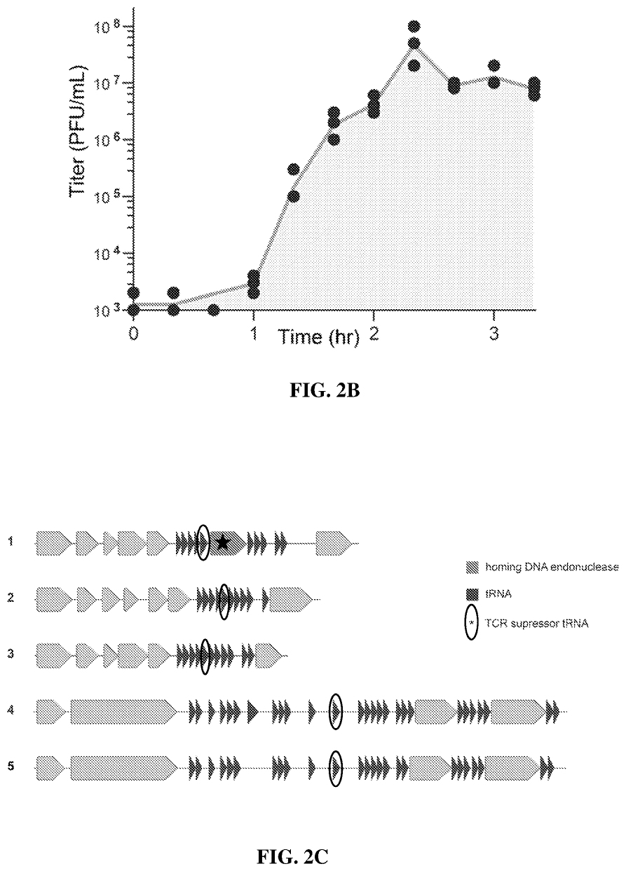

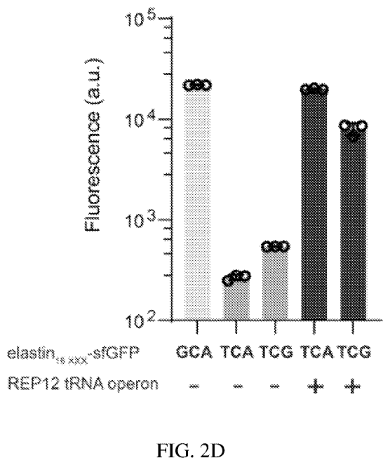

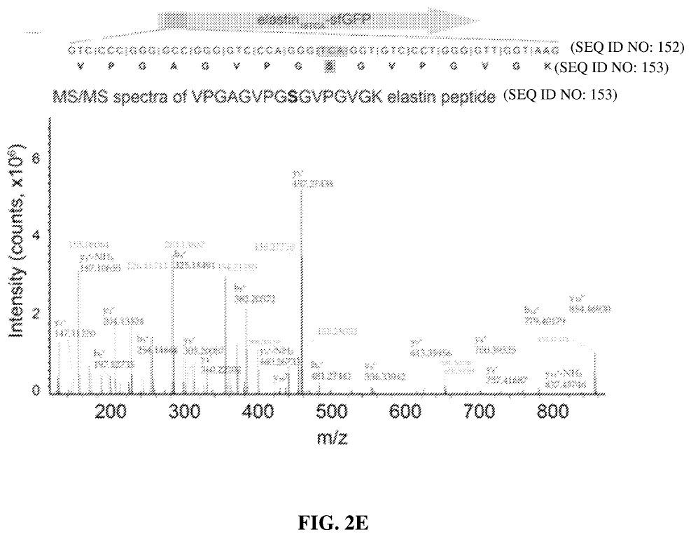

We then discovered twelve lytic viruses in environmental samples that can lyse E. coli Syn61Δ3 (FIGS. 2A-2B). These bacteriophages harbor and express up to 27 tRNA genes, including a functional tRNA-SerUGA needed to overcome the host's genetic-code-based virus resistance. These findings impact currently ongoing recoding projects, including our aim to engineer a TAG stop codon recoded human GRO and a 57-codon recoded strain of E. coli, as some of the identified viral tRNAs might enable viral replication in these recoded organisms (data not shown). Therefore, we are now implementing additional genetic firewalls to ensure the complete virus resistance of these engineered hosts.

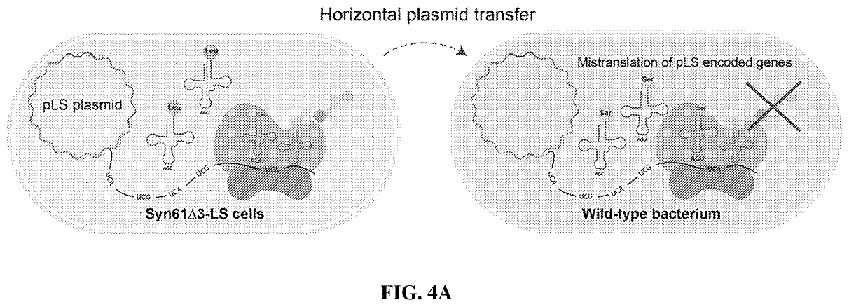

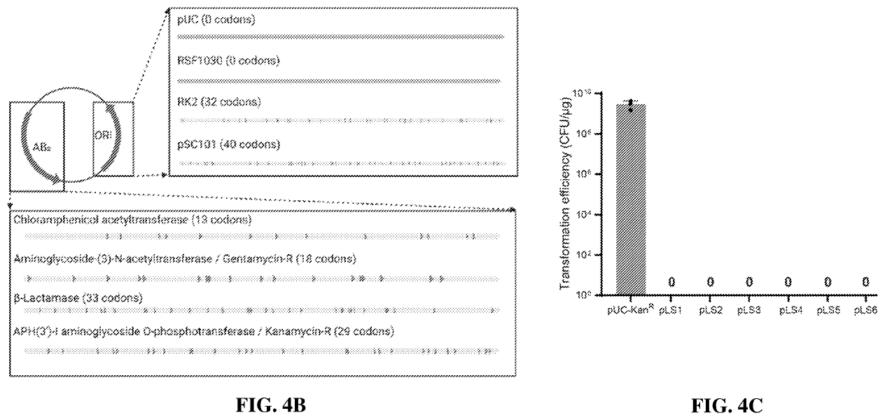

Finally, we have created a biocontained E. coli GRO, Ec_Syn61Δ3-SL, with an artificial genetic code that resists infection by a wide range of bacteriophages, including phages in raw sewage and the most virulent tRNA-encoding isolates of this study. Ec_Syn61Δ3-SL achieves this remarkable virus resistance by the engineered reassignment of TCR codons to leucine—an amino acid different from their natural serine identity—and thus mistranslating viral proteomes and mobile genetic elements that rely on the standard genetic code. Consequently, the genetic code of Ec_Syn61Δ3-SL poses a bidirectional genetic firewall that simultaneously prevents viral replication inside and the escape of synthetic genetic information from Ec_Syn61Δ3-SL into natural ecosystems. By addicting plasmids to express leucine-containing proteins with TCR codons, we also developed a set of vectors that cannot function in cells bearing the canonical genetic code. These plasmids, called the pLS plasmid series (FIGS. 4A-4B), restrict any synthetic constructs' functionality to Ec_Syn61Δ3-SL cells and thus provide escape-free containment for engineered genetic information.

We expect that these results will have broad implications on the safe use of Genetically Modified Organisms in open environments by establishing a generalizable method for genetic code alteration in GROs that simultaneously prevents viral predation in natural ecosystems and blocks incoming and outgoing HGT with natural organisms. The combination of genome recoding and codon reassignment might provide a universal strategy to make any species resistant to all natural viruses.

### Example 1. Mobile tRNA Genes Participate in Translation and Facilitate Horizontal Gene Transfer

We first investigated whether mobile genetic element-encoded tRNAs can complement cellular tRNAs and support viral infection in cells with a compressed genetic code. We sampled the mobile tRNAome, tRNA genes encoded by horizontally transferred genetic elements, by selecting and synthesizing 1192 tRNA genes from phylogenetically diverse plasmids, transposable elements, and bacteriophages infecting members of the Enterobacteriaceae family (data not shown). Next, we assayed these tRNAs for their ability to produce functional tRNAs in an E. coli host and substitute genomic tRNA genes to translate TCR codons. As depicted in FIG. 1A, this assay relies on an E. coli strain with a synthetic recoded genome in which all annotated instances of two sense serine codons (TCG, TCA) and a stop codon (TAG) were replaced with synonymous alternatives, and the corresponding serU, serT tRNA genes and release factor 1 (prfA) have been deleted. This strain, E. coli Syn61Δ3, thereby relies on a 61-codon genetic code and prevents the expression of protein-coding genes containing TCR codons. Candidate tRNAs have been synthesized and cloned into a plasmid carrying each tRNA under a strong constitutive promoter together with an nptII_{40TcA,68TcG,104TCA,251TCG} aph(3′)-II aminoglycoside-O-phosphotransferase antibiotic resistance gene containing TCA codons at positions 40, 104 and TCG codons at positions 68 and 251. In wild-type E. coli cells bearing the canonical genetic code, nptII_{40TCA,68TCG,104TCA,251TCG} confers resistance to kanamycin through serine incorporation at positions 40, 68, 104, and 251, and the production of full-length aph(3′)-II aminoglycoside-O-phosphotransferase, but in Syn61Δ3, the production of this resistance-conferring gene product is inhibited due to the lack of serU and serT-encoded tRNA-Ser_{UGA} and -Ser_{CGA} needed for TCR codon decoding. Therefore, in our screen, only plasmid variants that are expressing tRNAs capable of decoding TCR codons will survive kanamycin selection. The transformation of this plasmid library into Syn61Δ3 and subsequent selection in the presence of kanamycin yielded thousands of colonies, indicating the presence of TCR suppressor tRNAs in our library. Pooled extraction of plasmid variants from kanamycin-resistant colonies followed by amplicon sequencing of their tRNA-insert identified 61 tRNA sequences capable of promoting nptII_{40TCA,68TCG,104TCA,251TCG} expression (FIG. 1B). These tRNAs represent 89% of all predicted TCR codon-recognizing tRNAs in our library and share 61.1-33.7% (median=46.2%) similarity to the endogenous serU tRNA of E. coli. In agreement with the anticodon composition of mobile Ser tRNAs, most tRNA hits contained a UGA anticodon and carried the identity elements necessary for recognition by the host's SerS serine-tRNA-ligase (FIG. 1B).

Notable examples include the UAG anticodon-containing serine tRNA of the laboratory model coliphage T5, tRNAs from plasmids of multidrug-resistant E. coli isolates (GenBank IDs AP018804 and CP023851), and the Ser-tRNA_{CGA} of the integrative conjugative element of Acidithiobacillus ferrooxidans. The presence of mobile tRNAs in integrative conjugative elements is especially concerning as these mobile genetic elements can carry up to 38 tRNAs corresponding to all 20 amino acids in a single operon and are capable of excision and transfer into neighboring bacterial cells. In agreement with prior studies, our computational screen also showed that mobile tRNA genes are not limited to mobile genetic elements of bacteria. Computational analysis of viruses infecting Vertebrates and Archaea highlighted the presence of sense and stop codon suppressor tRNA encoding genes in both groups, suggesting that mobile tRNAs are prevalent across viruses infecting Prokaryotic, Archaeal, and Eukaryotic hosts (data not shown).

We confirmed the TCR codon-recognizing tRNAs' predicted serine amino acid identity by coexpressing selected tRNA hits with an elastin_{16TCA}-sfGFP-6×HIS construct harboring a single TCA codon at position 16. Tryptic digest followed by liquid chromatography and tandem mass spectrometry (LC/MS-MS) confirmed serine incorporation at the TCA position for all tested tRNAs (FIG. 1C).

Next, we investigated whether mobile tRNAome-derived tRNAs could promote viral replication. A previous study demonstrated that Syn61Δ3 resists infection by multiple bacteriophages, including Enterobacteria phage T6. Infecting Syn61Δ3 with T6 phage recapitulated these results. In contrast, the infection of Syn61Δ3 harboring a bacteriophage-derived Ser-tRNA_{UGA} gene with T6 resulted in rapid lysis, indicating that tRNA genes that reside in viral genomes can substitute cellular tRNAs and promote phage infection (FIG. 1D).

These results indicate that mobile tRNA genes are widespread and readily complement the lack of cellular tRNAs to promote viral replication and horizontal gene transfer.

### Example 2. Isolation of Lytic Viruses Infecting Syn61Δ3

We next investigated whether lytic viruses of Syn61Δ3 exist. We infected Syn61Δ3 cells with eleven coliphages whose genome harbor TCR suppressor tRNA genes according to our earlier plasmid-based screen (FIG. 1B). Surprisingly, none of these eleven phages could overcome the recoded host's genetic isolation, indicating that the presence of tRNA genes on viral genomes does not directly translate into viral replication in recoded organisms (data not shown).

We next attempted to isolate lytic viruses from diverse environmental samples by performing a standard two-step enrichment-based phage isolation protocol and using Syn61Δ3 as host. First, bacteria-free filtrates of environmental and wastewater samples (n=13, from Massachusetts (USA), data not shown) were mixed with Syn61Δ3 and grown until stationary phase. Next, bacterial cells were removed, and we analyzed the presence of lytic phages by mixing sample supernatants with Syn61Δ3 in soft-agar overlays. Five samples produced visible lysis. Viral plaque isolation from these samples followed by DNA sequencing and de novo genome assembly identified 12 distinct strains. All identified phages belong to the Caudovirales order and the Myoviridae family, taxa rich in tRNA-encoding bacteriophages (data not shown). Computational identification of tRNA genes revealed the presence of tRNA gene operons in all phage isolates, with 10 to 27 tRNA genes in each genome. Surprisingly, all isolates harbored TCR suppressor serine tRNAs with a UGA anticodon that we identified in our earlier nptII_{40TCA,68TCG,104TCA,251TCG} suppressor screen (FIG. 1B). One isolate, S3_1, also harbored a predicted homing endonuclease within its tRNA operon (FIGS. 2A-2C). Homing endonucleases encoded in tRNA operons have been shown to be responsible for the horizontal transfer of tRNA gene clusters within phage genomes.

Lytic phages of Syn61Δ3 showed more than two orders of magnitude difference in viral titers after growth on recoded cells. One of the most virulent isolates, S13_4, required 60 minutes to complete a replication cycle at 37° C. in Syn61Δ3.

The isolated viral strains infecting Syn61Δ3 show that bacteriophages that can overcome sense codon recoding-based viral resistance exist and are widespread in environmental samples.

### Example 3. Viral tRNAs Substitute Cellular tRNAs to Support Translation

We next investigated how tRNA-encoding viruses evade genetic-code-based resistance. Time-course transcriptome analysis of infected Syn61Δ3 cells during the viral replication cycle revealed early and high-level expression of the viral tRNA operon (data not shown). In agreement with this observation, the computational prediction of bacterial promoters driving the tRNA array indicated the presence of multiple strong constitutive promoters upstream of the tRNA operon region (data not shown). We then investigated the time-course kinetics of tRNA expression in Syn61Δ3 cell that were infected with our S134 phage by performing tRNA sequencing (tRNAseq). Time-course tRNAseq experiments revealed remarkably high-level expression of the viral tRNA-Ser_{UGA} immediately after phage attachment (i.e., a relative viral tRNA-Ser_{UGA} abundance of 56.1% (±5%) compared to the host serV tRNA). Throughout the entire phage replication cycle, the phage tRNA-Ser_{UGA} remained one of the most abundant viral tRNA species inside infected Syn61Δ3 cells (data not shown). We next investigated whether phage tRNA-Ser_{UGA} participate in translation by analyzing the presence of their mature form. The gene encoding the tRNA-Ser_{UGA} in S13_4's genome does not encode the universal 5′-CCA tRNA tail, which allows for amino acid attachment as well as for interaction with the ribosome. Therefore, CCA tail addition must happen before these tRNAs can participate in translational processes. The sequencing-based analysis of phage tRNA-Ser_{UGA} ends detected CCA tail addition in 62.9% (±1.9%) of all tRNA sequencing reads immediately after phage attachment, indicating that mature tRNA-Ser_{UGA}s are instantly being produced after host infection (data not shown).

We have also investigated transcriptomic changes in Syn61Δ3 during phage replication. Analysis of the host transcriptome after phage infection revealed upregulation in genes responsible for tRNA maturation and modification. Upregulated genes include queG, encoding epoxyqueuosine reductase that catalyzes the final step in the de novo synthesis of queuosine in tRNAs, and trmJ, tRNA C^{m32}/U^{m32} methyltransferase, which introduces methyl groups at the 2′-O position of U³² of several tRNAs, including tRNA-Ser_{UGA}, suggesting the potential posttranscriptional modification of phage-derived tRNAs (data not shown). Finally, we also validated the role of phage tRNA-SeruGAs in decoding TCR codons. We first cloned the viral tRNA operon containing the hypothetical tRNA-Ser_{UGA} and its predicted promoter to identify the tRNA responsible for decoding TCR codons in viral proteins. Coexpression of this tRNA operon with an elastin_{16TCA}-sfGFP-6×HIS and elastin_{16TCG}-sfGFP-6×HIS construct harboring either a single TCA or TCG codon at position 16, respectively, resulted in near wild-type level production of elastin-sfGFP-6×HIS (FIG. 2D). Furthermore, tryptic digestion followed by LC/MS-MS analysis confirmed serine incorporation in response to both the TCA and TCG codon in these samples (FIG. 2E). Finally, the coexpression of the same elastin16TCA-sfGFP-6×HIS construct with the only tRNA-Ser_{UGA} of the viral tRNA operon conferred a similar effect, and LC/MS-MS analysis confirmed the role of this tRNA in decoding viral TCR codons as serine (data not shown).

Together these results show that lytic phages of Syn61Δ3 overcome genetic-code-based viral resistance by rapidly complementing the cellular tRNA pool with virus-encoded tRNAs.

### Example 4. Creation of an Amino-Acid-Swapped Genetic Code

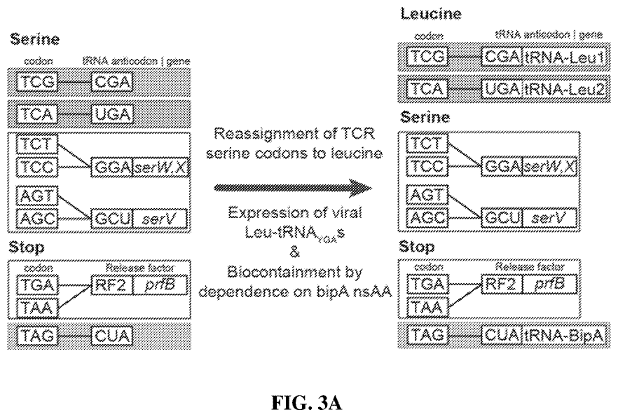

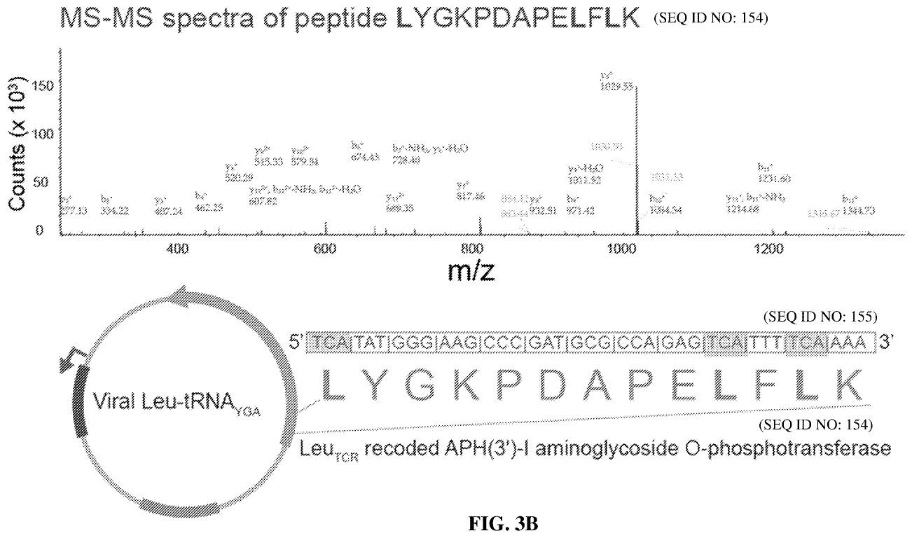

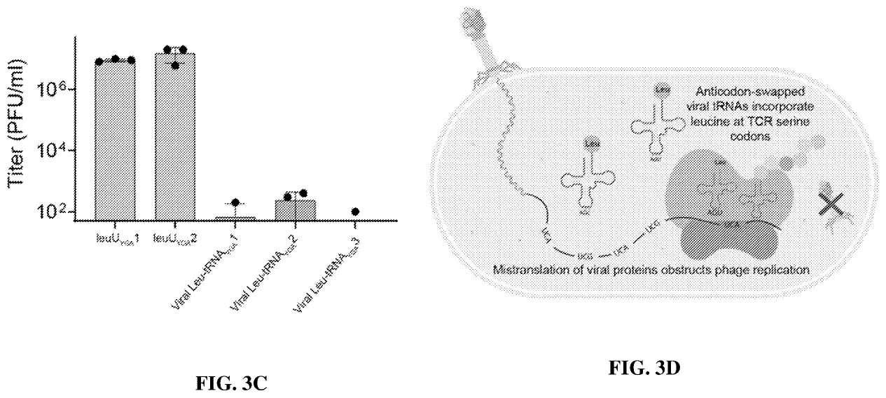

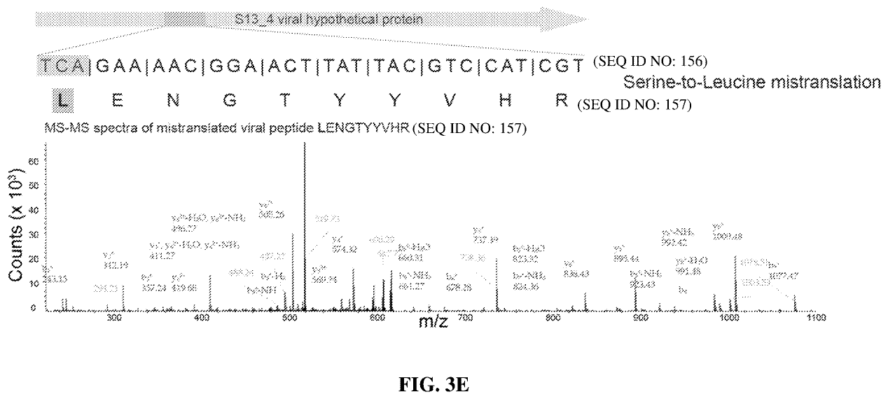

We predicted that establishing an artificial genetic code, in which TCR codons encode an amino acid different from their natural serine identity, would create a genetic firewall that safeguards cells from horizontal gene transfer and infection by tRNA-encoding viruses. In an amino acid swapped genetic code, viral tRNAs would compete with host-expressed tRNAs that decode TCR codons as a non-serine amino acid resulting in the severe mistranslation of viral proteins. Although swapping the amino acid identity of sense codons presents a possible way to prevent horizontal gene transfer, it was impossible to test this hypothesis in vivo until now. To establish a serine_{TCR}-to-leucine swapped genetic code (FIG. 3A), we utilized Syn61Δ3, which genome-wide lacks annotated instances of TCR codons and their corresponding tRNA genes and sought to identify tRNAs capable of efficiently translating TCR codons as leucine. To this aim, we modified our previous tRNA library selection screen (FIG. 1A) to evolve efficient TCR suppressors from the endogenous E. coli leuU tRNA carrying a TCA and TCG decoding anticodon. We coexpressed a 65,536-member mutagenized library of the anticodon-swapped leuU tRNA gene in which the anticodon loop of both tRNAs have been fully randomized, together with an aph31a_{29×Leu→TCR}, a kanamycin resistance-conferring gene in which all 29 instances of leucine codons were replaced with TCR serine codons. In this system, only anticodon-swapped leuU variants capable of translating all 29 TCR codons as leucine would confer resistance to kanamycin. We identified two distinct leuU variants by applying “high” kanamycin concentration (i.e., 200 g/ml) as selection pressure to Syn61Δ3 cells carrying the anticodon-swapped tRNA library. These variants, carrying tRNAs containing distinct anticodon loop mutations, were then infected with a cocktail of all twelve phage isolates that are capable of lysing Syn61Δ3 at a 10:1 cell-to-phage ratio (i.e., a Multiplicity of Infection (MOI) of 0.1). Surprisingly, all selected leuU library members allowed robust phage replication, with phage titers reaching ~10⁷ PFU/ml after 24 hours (FIG. 3C). We hypothesized that viral replication in the presence of TCR suppressing leuU variants is due to these tRNAs lower suppression efficiency compared to phage-carried serine tRNAs, which leads to rapid viral takeover. Viral tRNA-Ser_{Y}GA, that are tRNA-Ser_{UGA} and tRNA-Ser_{CGA}, might have: i.) higher aminoacylation efficiency by their corresponding E. coli aminoacyl-tRNA-ligase than our selected leuU variants, ii.) higher affinity towards the bacterial ribosome, and/or iii.) better evade phage- and host-carried tRNA-degrading effector proteins.

Based on this observation, we hypothesized that bacteriophage-encoded tRNAs might provide higher suppression efficiencies for their cognate codons than their native E. coli counterpart. Therefore, we next constructed a small, focused library that coexpressed YGA anticodon swapped mutants of 13 phage-encoded leucine tRNAs, together with our previous aph31a_{29×Leu→TCR} gene in which all 29 instances of leucine coding codons were replaced with TCR serine codons. The transformation of this library into Syn61Δ3 cells and subsequent “high” concentration (i.e., 200 g/ml) kanamycin selection identified three distinct variants displaying robust growth. Identified tRNAs showed 48.3-37.9% similarity to E. coli leuU but carried most of the canonical leuS E. coli leucine-tRNA ligase identity elements. Furthermore, the analysis of the total tRNA content of these cells by tRNAseq confirmed the presence of synthetic phage Leu-tRNA_{Y}GA tRNAs with similar abundances as the cellular endogenous serine tRNAs (i.e., a relative expression level of 172% and 140% for Leu-tRNA_{UGA} and Leu-tRNA_{CGA} respectively, compared to serV (data not shown).

Next, similarly to our previous infection assay, phage tRNA_{Y}GA expressing cells were infected with a mixture of twelve distinct, lytic phages of Syn61Δ3 at a MOI=0.1. The analysis of phage titer in culture supernatants after 24 hours showed a marked drop compared to the input phage inoculum, suggesting that anticodon-swapped viral leucine tRNAs block phage replication (FIG. 3C).

We then investigated the mechanism of phage resistance in E. coli cells carrying phage-derived tRNA-Leu_{YGA} tRNAs (Ec_Syn61Δ3 Ser>Leu Swap, or Ec_Syn61Δ3-SL in short) by performing total proteome analysis. Untargeted proteome analysis of uninfected cells by tandem mass spectrometry validated the translation of TCR codons as leucine in Ec_Syn61Δ3-SL (FIG. 3B). Time-course untargeted proteome analysis after bacteriophage infection revealed extensive mistranslation at TCR codons in newly synthesized phage proteins (FIG. 3E), indicating that an amino acid swapped genetic code broadly obstructs viral protein synthesis. In agreement with earlier reports that showed the partial recognition of TCT codons by tRNA_{UGA}, we also detected serine-to-leucine mistranslation at TCT codon positions in Ec_Syn61Δ3-SL cells (data not shown). The recognition of TCT codons by phage tRNA-Leu_{YGA} tRNAs might also be responsible for the slight fitness decrease of Ec_Syn61Δ3-SL cells compared to its ancestor strain (i.e., a doubling time of 69.3 minutes, compared to 44.29 minutes for the parental Syn61Δ3 ΔrecA (ev1) in rich 2×YT media (data not shown). Alternatively, the fitness decrease of Ec_Syn61Δ3-SL might be also attributable to the presence of nonrecoded TCR codons in essential genes of Syn61Δ3. According to our genome analysis, at least four essential genes of Syn61Δ3, mukE, ykjM, yjbS, safA, contain TCR codons and became mistranslated in Ec_Syn61Δ3-SL (data not shown).

Finally, we also sought to develop a tightly biocontained version of Ec_Syn61Δ3-SL because a virus-resistant strain might have competitive advantage in natural ecosystems due to lack of predating bacteriophages. Synthetic auxotrophy based on the engineered reliance of essential proteins on human-provided nonstandard amino acids, e.g., L-4,4′-biphenylalanine (bipA), offers tight, likely escape-free biocontainment that remains stable under long-term evolution. Therefore, we generated a recombination deficient, biocontained version of Ec_Syn61Δ3-SL bearing a bipA-dependent essential adk gene and the bipA aminoacyl-tRNA synthetase/tRNA-bipA_{CUA} system using Cas9-assisted multiplexed ssDNA recombineering. This strain maintained the low escape rate of previously reported synthetic auxotrophs and provided robust growth. We also tested the viral resistance of the resulted biocontained Ec_Syn61Δ3-SL strain under mock environmental conditions by repeating our phage enrichment and isolation process with a mixture of 12 environmental samples, including raw sewage, but could not detect lytic phages in culture supernatants (data not shown).

Together, these results demonstrate that reassigning sense codons TCA and TCG to leucine in vivo provides multivirus resistance and the TAG stop codon of can be simultaneously utilized to provide tight biocontainment.

### Example 5. Addiction to an Amino-Acid-Swapped Genetic Code Provides a Bidirectional Firewall for Synthetic Genetic Information

Finally, we developed a set of plasmid vectors that we systematically addicted to an artificial genetic code in which leucine is encoded as TCR codons. Genetically Modified Organisms (GMOs) are increasingly deployed for large-scale use in agriculture, therapeutics, bioenergy, and bioremediation. Consequently, it is critical to implement robust biocontainment strategies that prevent the unintended proliferation of GMOs and protect natural ecosystems from engineered genetic information. Although efficient biocontainment strategies for GMOs exist (e.g., bipA nsAA-based synthetic auxotrophy, as in Ec_Syn61Δ3-SL), current methods fail to prevent the horizontal gene transfer (HGT)-based escape of engineered genetic information. Synthetic addiction to an artificial genetic code offers a solution to this problem. Using our phage-derived tRNA-Leu_{Y}GA expressing Ec_Syn61Δ3-SL cells, we, therefore, developed a set of plasmid vectors that depend on TCR codons to express leucine-containing proteins and thus can only function in cells that efficiently translate TCR codons as leucine (FIG. 4A). These plasmids, called the pLS plasmids, offer four orthogonal antibiotic resistance markers in combination with four mutually orthogonal low- to high-copy-number origins-of-replication for stable maintenance in Ec_Syn61Δ3-SL cells (FIG. 4B). Antibiotic resistance genes and proteins necessary for pLS plasmid replication encode leucine as TCR-naturally serine-meaning-codons and, therefore, fail to function in cells bearing the canonical genetic code. The addiction of resistance markers and replication proteins to an artificial genetic code ensures that pLS plasmids can stably and safely maintain synthetic genetic functions but restrict these genes' functionality to Ec_Syn61Δ3-SL cells. We tested the ability of our pLS vectors to function in cells bearing the standard genetic code cells by transforming six variants into wild-type E. coli K-12 MG1655 cells but failed to detect escapees carrying functional plasmids. The escape of pLS plasmids was similarly prevented when the phage tRNA-Leu_{Y}GA expression cassette was encoded within the plasmid backbone, indicating that anticodon-swapped tRNAs are severely toxic to wild-type cells (FIG. 4C). Based on these results we also expect that similarly to pLS' genes, any leucine-containing protein can be addicted to Ec_Syn61Δ3-SL by recoding target genes to encode one or more leucine positions as TCR codons.

In sum, the addiction of pLS plasmids to an artificial genetic code in which leucine is encoded as TCR codons, in combination with nsAA-based synthetic auxotrophy, offers escape-free biocontainment for engineered genetic information.

### Methods

### Bacterial Media and Reagents

Lysogeny Broth Lennox (LBL) was prepared by dissolving 10 g/l tryptone, 5 g/l yeast extract, and 5 g/l sodium chloride in deionized H₂O and sterilized by autoclaving. Super Optimal Broth (SOB) was prepared by dissolving 20 g/l tryptone, 5 g/l yeast extract, 0.5 g/l sodium chloride, 2.4 g/l magnesium sulfate and 0.186 g/l potassium chloride in deionized H₂O and sterilized by autoclaving. 2×YT media consisted of 16 g/l casein digest peptone, 10 g/l yeast extract, 5 g/l sodium chloride. LBL and 2×YT agar plates were prepared by supplementing LBL medium or 2×YT with agar at 1.6% w/v before autoclaving. Top agar for agar overlay assays was prepared by supplementing LBL medium with agarose at 0.7% w/v before autoclaving. SM Buffer, 50 mM Tris-HCl (pH 7.5), 100 mM NaCl, 8 mM MgSO₄, 0.01% gelatin, was used for storing and diluting bacteriophage stocks (Geno Technology, Inc., St. Louis, MO, USA). L-4,4′-biphenylalanine (bipA) was obtained from PepTech Corporation (USA).

### Bacteriophage Isolation

Bacteriophages were isolated from environmental samples from Greater Massachusetts, United States by using E. coli Syn61Δ3(ev5) (from the laboratory of Jason W. Chin (Addgene strain #174514)) as host. For aqueous samples, including sewage, we directly used 50 ml filter-sterilized filtrates, while samples with mainly solid components, like soil and animal feces, were first resuspended to release phage particles and then sterilized by centrifugation and subsequent filtration. This protocol avoided the inactivation of chloroform sensitive viruses. Sterilized samples were then mixed with exponentially growing cultures of Syn61Δ3(ev5) in SOB supplemented with 10 mM CaCl2) and MgCl2. Infected cultures were grown overnight at 37° C. aerobically and then filter sterilized by centrifugation at 4000×g for 15 minutes and filtered through a 0.45 m PVDF Steriflip™ disposable vacuum filter unit (MilliporeSigma). Next, 1 ml from each sterilized enriched culture was mixed with 10 ml exponentially growing Syn61Δ3 (OD₆₀₀=0.2), supplemented with 10 mM CaCl2) and MgCl2, and mixed with 10 ml 0.7% LBL top agar. Top agar suspensions were then poured on top of LBL agar plates in 145×20 mm Petri dishes (Greiner Bio-One). Petri dishes were incubated overnight at 37° C. and inspected for phage plaques on the next day. Areas with visible lysis or plaques were excised, resuspended in SM buffer, and diluted to single plaques on top agar lawns containing 99% Syn61Δ3 and 1% MDS42 cells. We note that adding trace amounts of MDS42 cells greatly increased the visibility of plaques and clear plaques could be easily picked indicating phage replication on the recoded host. Dilutions and single plaque isolations were repeated four times for each plaque to purify isogenic phages. Finally, high-titer stocks were prepared by mixing sterilized suspensions from single plaques with exponentially growing MDS42 cells (OD₆₀₀=0.3) in SOB supplemented with 10 mM CaCl2) and MgCl2. Phage infected samples were grown at 37° C. until complete lysis (~4 hrs) and then sterilized by filtration.

### Bacteriophage Culturing

Bacteriophage stocks were prepared by a modified liquid lysate Phages on Tap protocol in LBL medium. High-titer lysates were prepared from single plaques by picking well isolated phages plaques into SM buffer and then seeding 3-50 ml early exponential phase cultures of E. coli MDS42 cells in SOB broth supplemented with 10 mM CaCl₂) and MgCl₂. Phage infected samples were grown at 37° C. until complete lysis and then sterilized by filtration. High-titer phage lysates were stored at 4° C. in the dark and phages were archived as virocells and stored at −80° C. in the presence of 25% glycerol for long-term storage.

### Phage Replication Assay

Genomic TCR-suppressor tRNA-Ser_{UGA} gene containing phages, corresponding to NCBI GenBank numbers MZ501046, MZ501058, MZ501065, MZ501066, MZ501067, MZ501074, MZ501075, MZ501089, MZ501096, MZ501098, MZ501105, MZ501106, were obtained from DSMZ (Germany). Exponential phase cultures (OD₆₀₀=0.3) of MDS42 and Syn61Δ3(ev5) were grown in SOB supplemented with 10 mM CaCl2) and MgCl2 at 37° C. Cultures were infected with phage at an MOI of approximately 0.001. Simultaneously, the same amount of each phage was added to sterile SOB broth supplemented with 10 mM CaCl2) and MgCl2 to act as a cell-free control for input phage calculation. Infected cultures were grown at 37° C. with shaking at 250 rpm. After 24 hours, cultures were transferred to 1 ml tubes and centrifuged at 19,000×g to remove cells and cellular debris, and the clarified supernatant was serially diluted in SM buffer to enumerate output phage concentration. 1.5 μl of the diluted supernatants were applied to LBL 0.7% top agar seeded with MDS42 cells and 10 mM CaCl2) and MgCl2 using a 96 fixed pin multi-blot replicator (VP407, V&P Scientific). Following 18 hours of incubation at 37° C., plaques were counted, and the number of plaques were multiplied by the dilution to calculate the phage titer of the original sample.

### Single-Step Phage Growth Curve

An exponential phase culture (OD₆₀₀=0.3) of Syn61Δ3 was grown in 50 ml SOB broth supplemented with 10 mM CaCl2) and MgCl2 at 37° C. with shaking at 250 rpm. Cultures were then spun down and resuspended in 3 ml SOB supplemented with 10 mM CaCl2) and MgCl2 and 1 ml samples were infected with S134 phage at an MOI of 0.01. Infected cultures were incubated at 37° C. without shaking for phage attachment and then washed twice with 1 ml SOB broth by pelleting cells at 4000×g for 3 minutes. Infected cells were then diluted into 50 ml SOB supplemented with 10 mM CaCl2) and MgCl2 and incubated at 37° C. with shaking at 250 rpm. At every 20 minutes, 1 ml sample was measured out into a sterile Eppendorf tube containing 100 μl chloroform, immediately vortexed, and then placed on ice. Phage titers were determined by centrifuging chloroformed cultures at 6000×g for 3 minutes and then serially diluting supernatants in SM buffer and spotting 1 μl dilutions to LBL 0.7% top agar plates seeded with MDS42 cells and 10 mM CaCl2) and MgCl2. Following 18 hours of incubation at 37° C., plaques were counted, and the number of plaques were multiplied by the dilution to calculate the phage titer of the original sample.

### Bacteriophage Genome Sequencing, Assembly, and Annotation

Genomic DNA of bacteriophages was prepared from high-titer (i.e., >10¹⁰ PFU/mL) stocks after DNase treatment using the Norgen Biotek Phage DNA Isolation Kit (Cat #46800) according to the manufacturer's guidelines and sequenced at the Microbial Genome Sequencing Center (MiGS, Pittsburgh, PA, USA). Sequencing libraries were prepared using the Illumina DNA Prep kit and IDT 10 bp UDI indices, and sequenced on an Illumina NextSeq 2000, producing 150 bp paired end reads. Demultiplexing, quality control and adapter trimming was performed with bcl-convert (v3.9.3). Reads were trimmed to Q28 using BBDuk from BBTools. Phage genomes were then assembled de novo using SPAdes 3.15.2 in—careful mode with an average read coverage of 10-50×. Assembled genomes were then annotated by using Prokka version 1.14.6 with default parameters except that the PHROGs HMM database was used as input to improve phage functional gene annotations.

### Bacterial Genome Sequencing and Annotation

Genomic DNA from overnight saturated cultures of isogenic bacterial clones was prepared using the MasterPure™ Complete DNA and RNA Purification Kit (Lucigen) according to the manufacturer's guidelines and sequenced at the Microbial Genome Sequencing Center (MiGS, Pittsburgh, PA, USA). Sequencing libraries were prepared using the Illumina DNA Prep kit and IDT 10 bp UDI indices, and sequenced on an Illumina NextSeq 2000, producing 150 bp paired end reads. Demultiplexing, quality control and adapter trimming was performed with bcl-convert (v3.9.3). Reads were then trimmed to Q28 using BBDuk from BBTools and aligned to their corresponding reference by using Bowtie2 2.3.0 in—sensitive-local mode. Single-nucleotide polymorphisms (SNPs) and indels were called using breseq (version 0.36.1). Only variants with prevalence higher than 75% were voted as mutations. Following variant calling, mutations were also manually inspected within the aligned sequencing reads in all cases. The de novo sequencing and genome assembly of Syn61Δ3(ev5) (from on a single-colony isolate of Addgene strain #174514) was performed by generating 84,136 Oxford Nanopore (ONT) long-reads by PCR-free library generation (Oxford Nanopore, UK) on a MinION Flow Cell (R9.4.1) and 4.5×10⁶ 150 bp paired-end reads on an Illumina NextSeq 2000. Quality control and adapter trimming was performed with bcl2fastq 2.20.0.445 and porechop 0.2.3_seqan2.1.1 for Illumina and ONT sequencing, respectively. Next, we performed hybrid assembly with Illumina and ONT reads by using Unicycler 0.4.8 (default parameters). Finally, the resulted single, circular contig representing the entire genome was manually inspected for errors in Geneious Prime® 2022.1.1. and annotated based on sequence homology by using the BLAST function implemented in Geneious Prime® 2022.1.1. based on E. coli K-12 MG1655 (NCBI ID: U00096.3) as reference. Gene essentiality was determined based on.

### Transcriptome Analysis of Phage Infected Cells

We explored transcriptomic changes and mRNA production in phage infected Syn61Δ3 cells by performing a modified single-step growth experiment and collected samples at 20 minutes intervals. 50 ml of early-exponential (OD₆₀₀=0.15) Syn61Δ3 cells (corresponding to 2×10¹⁰ CFU) growing at 37° C., 250 rpm in SOB containing 10 mM CaCl2) and MgCl2 were spun down at room temperature and resuspended in 1 ml of SOB. 50 μl of this uninfected sample was immediately frozen in liquid N2 and stored at −80° C. until RNA extraction. Next, 900 μl of this cell suspension was mixed with 10 ml prewarmed S134 phage stock (i.e., ~7×10¹⁰ PFU to achieve a MOI, multiplicity of infection, of ~4) in SOB containing 10 mM CaCl2) and MgCl2, and then incubated at 37° C. for 10 minutes without shaking for phage absorption. Following phage attachment, samples were spun down, washed with 1 ml SOB twice to remove unabsorbed phages, and then resuspended in 10 ml SOB containing 10 mM CaCl2) and MgCl2. Samples were then incubated at 37° C., 250 rpm. After 20- and 40-minutes post-infection, we spun down 1 ml cell suspension from each sample and the cell pellets were frozen in liquid nitrogen and stored at −80° C. until RNA extraction. As expected, after 60 minutes post-infection no cell pellet was visible. Phage infections were performed in three independent replicates. Total RNA from frozen samples was extracted by using the RNeasy Mini Kit (Qiagen, USA) according to the manufacturer's instructions and the extracted RNA was DNAse treated with Invitrogen RNase-free DNAse (Thermo Fisher Scientific, USA). Sequencing library preparation was then performed using Stranded Total RNA Prep Ligation kit with Ribo-Zero Plus for rRNA depletion and by using 10 bp IDT for Illumina indices (all from Illumina, USA). Sequencing was done on a NextSeq2000 instrument in 2×50 bp paired-end mode. Demultiplexing, quality control, and adapter trimming was performed with bcl-convert (v3.9.3).

tRNA Sequencing Sample Preparation

We explored tRNA expression levels and changes in phage infected Syn61Δ3 cells by performing a modified single-step growth experiment with an increased MOI and cell mass to increase extracted tRNA amounts for successful sequencing library generation. Early-exponential (OD₆₀₀=0.2) Syn61Δ3 cells (corresponding to approximately 5×10¹⁰ CFU) growing at 37° C., 250 rpm in SOB containing 10 mM CaCl2) and MgCl2 were spun down at room temperature and resuspended in 1.1 ml of SOB. 100 μl of this uninfected sample was immediately frozen in liquid N2 and stored at −80° C. until tRNA extraction. Next, 1000 ul of this cell suspension was mixed with 20 ml prewarmed S134 phage stock (i.e., ~10² PFU to achieve a MOI of ~20) in SOB containing 10 mM CaCl2) and MgCl2, and then incubated at 37° C. for 10 minutes without shaking for phage absorption. Following phage attachment, samples were spun down, the supernatant containing unabsorbed phages was removed, and the cell pellet was then resuspended in 7 ml SOB containing 10 mM CaCl2) and MgCl2. Samples were then incubated at 37° C., 250 rpm. Immediately after phage attachment and after 20- and 40-minutes post-infection, 1 ml cell suspensions from each sample were spun down, and cell pellets were frozen in liquid N2 and stored at −80° C. until total RNA extraction. Phage infections were performed in two independent replicates.

We analyzed of the total tRNA content of Ec_Syn61Δ3-SL cells expressing KP869110.1 viral tRNA24-Leu_{UGA} and tRNA24-Leu_{CGA} by pelleting cells from 5 ml mid-exponential (OD₆₀₀=0.3) culture at 4000×g and flash-freezing the cell pellet in liquid nitrogen.

We extracted tRNAs by lysing samples at room temperature (RT) for 30 mins in 150 μl lysis buffer containing 8 mg/mL lysozyme (from chicken egg white, #76346-678, VWR, USA), 10 mM Tris HCl pH 7.5, and 1 μl murine RNase inhibitor (New England Biolabs). Samples were then mixed with 700 μl Qiazol reagent (#79306, Qiagen) and incubated for 5 minutes at RT. Next, 150 μl chloroform was added, vortexed, and incubated until phase-separation. Samples were then spun at 15,000×g for 15 min at in a cooled centrifuge. The supernatant was transferred into a fresh tube and mixed with 350 μl 170% ethanol. Larger RNA molecules were then bound to a RNeasy MinElute spin column (#74204, Qiagen), and the flow-through was mixed with 450 μl of 100% ethanol and tRNAs were bound to a new RNeasy MinElute spin column. The tRNA fraction was then washed first with 500 μl wash buffer (#74204, Qiagen), next with 80% ethanol, and then eluted in RNase-free water. The eluted tRNAs were deacylated in 60 mM pH 9.5 borate buffer (J62154-AK, Alfa Aesar, Thermo Fisher Scientific) for 30 minutes and then purified using a Micro Bio-Spin P-30 Gel Column (7326251, from Bio-Rad).

tRNA Sequencing Library Preparation, Sequencing, and Data Analysis

We prepared tRNA cDNA libraries by reverse-transcribing tRNAs using the TGIRT™-III template-switching reverse-transcriptase (TGIRT50, InGex, USA) according to the manufacturer's instructions. In brief, we prepared reaction mixtures containing 1 μl (~100 g) of the deacylated tRNAs, 2 μl of 1 μM TGIRT DNA/RNA heteroduplex (prepared by hybridizing equimolar amounts of rCrUrUrUrGrArGrCrCrUrArArUrGrCrCrUrGrArArArGrArUrCrGrGrArArGrArGrCrArCrArC rGrUrCrUrArGrUrUrCrUrArCrArGrUrCrCrGrArCrGrArU/3SpC3/(SEQ ID NO: 7) and ATCGTCGGACTGTAGAACTAGACGTGTGCTCTTCCGATCTTTCAGGCATTAGGCTCA AAGN (SEQ ID NO: 8) oligos), 4 μl 5×TGIRT™ reaction buffer (2.25 M NaCl, 25 mM MgCl2, 100 mM Tris-HCl, pH 7.5), 2 μl of 100 mM DTT, 9 μl RNase-free water, and 1 μl TGIRT-III, and incubated at room temperature for 30 minutes to initiate template-switching. Next, 1 μl of 25 mM dNTPs (Thermo Fisher Scientific, USA) were added to the reaction mixture and samples were incubated at 60° C. for 30 minutes to perform reverse transcription. RNA was then hydrolyzed by NaOH, neutralized by HCl, and the cDNA library was purified using MinElute PCR purification kit. cDNAs were then ligated to a preadenylated DNA adapter/5Phos/GATCNNNAGATCGGAAGAGCGTCGTGT/3SpC3/(SEQ ID NO: 9), in which NNN denotes an N, NN, NNN spacer to increase library diversity during sequencing (preadenylated oligos were prepared by 5′ DNA adenylation kit (E2610L) using thermostable 5′ App DNA/RNA ligase (M0319L, both from New England Biolabs) following the manufacturer's protocol. The cDNA library was purified using the MinElute PCR purification kit (Qiagen) and amplified using Q5 Host-Start High-Fidelity 2× Master Mix (New England Biolabs). PCR products were then size selected to remove adaptor-dimers below 200 bp using three subsequent size-selection rounds with a Select-a-Size DNA Clean & Concentrator Kit (D4080, Zymo Research). Finally, amplicon libraries were barcoded using the using the IDT 10 bp UDI indices (Illumina) and sequenced on an Illumina MiSeq to produce 250 bp paired end reads. Read-demultiplexing was performed with bcl-convert (v3.9.3). Paired-end reads were then aligned to their reference sequences by using Geneious assembler, implemented in Geneious Prime® 2022.1.1, allowing a maximum of ten SNPs within tRNA reads compared to their reference. These settings allowed us to specifically map lower-fidelity TGIRT-III-transcribed cDNA reads to their corresponding reference sequence without cross-mapping to tRNAs sharing sequence homology. tRNA reads from Ec_Syn61Δ3-SL cells expressing KP869110.1 viral tRNA24 -Leu_{UGA} and tRNA24-Leu_{CGA} were mapped without allowing the presence of SNPs in sequencing reads to distinguish tRNA24-Leu_{UGA} and tRNA24-Leu_{CGA} that differs by only a single SNP within the anticodon region.

### Adaptive Laboratory Evolution of Syn61Δ3

Adaptive laboratory evolution experiments were performed in rich bacterial media for 30 days (~270 cell generations) on Syn61Δ3(ev5) ΔrecA cells to increase fitness. At each transfer step, 10⁹-10¹⁰ bacterial cells were transferred into 500 ml LBL medium containing 1.5 g/l Tris/Tris-HCl and incubated aerobically for 24 hours at 37° C., 250 rpm in a 2000 ml Erlenmeyer flask with vented cap. Following adaptation, cells were spread onto LBL agar plates, individual colonies were isolated, and then subjected to whole-genome sequencing.

### Doubling Time Measurements

To determine growth parameters under standard laboratory conditions, saturated overnight cultures of E. coli Syn61Δ3(ev5), Syn61Δ3(ev5) ΔrecA, and its evolved variant, Syn61Δ3(ev5) ΔrecA (ev1) were diluted 1:200 into 50 ml of 2×YT broth and LBL broth in a 300 ml Erlenmeyer flask with vented cap and incubated aerobically at 37° C., 250 rpm. Ec_Syn61Δ3-SL cells were characterized similarly, but by using 2×YT broth containing 50 g/ml kanamycin. All growth measurements were performed in triplicates. Optical density at 600 nm (OD₆₀₀) measurements were taken every 20 minutes for 8 hours or until stationary phase was reached on a C08000 Cell Density Meter, WPA. The doubling time was calculated for each independent replicates by log₂-transforming OD₆₀₀ values and calculating the doubling time based on every six consecutive data points during the exponential growth phase. We calculated the doubling time (1/slope) from a linear fit to log₂-derivatives of the six data points within this window and reported the shortest doubling time for each independent cultures. Curve fitting, linear regression, and doubling time calculations were performed with Prism9 (GraphPad). Error bars show ±standard deviation.

tRNA Annotation

We detected tRNA genes in the Viral genomic NCBI Reference Sequence Database (Accessed: Jan. 2, 2022) and in individual phage isolates' genome by using tRNAscan-SE 2.0.9 in bacterial (-B), archaeal (-A), or eukaryotic (-E) maximum sensitivity mode (-I—max). tRNAscan-SE detection parameters were chosen according to the predicted host of the corresponding viral isolate.

Mobile tRNAome tRNA Library Generation and Selection

We generated our mobile tRNAome expression library by synthesizing tRNAscan-SE predicted tRNAs from diverse sources, driven by a strong bacterial proK tRNA promoter and followed by two strong transcriptional terminators, as 10 pmol ssDNA oligonucleotide libraries (10 pmol oPool, from Integrated DNA Technologies, USA). Oligonucleotides were resuspended in 1×TE buffer and then amplified by 5′ phosphorylated primers. Amplicons were then blunt-end ligated into pCR4Blunt-TOPO (Invitrogen, Zero Blunt™ TOPO™ PCR Cloning Kit) for 18 hrs at 16° C. and then purified by using the Thermo Scientific GeneJET PCR Purification Kit. We then electroporated 50 ng purified plasmid in five parallel electroporations into 5×40 μl freshly made electrocompetent cells of MDS42 and Syn61Δ3(ev5). Prior to electrotransformation, bacterial cells were made electrocompetent by growing cells after a 1:100 dilution in SOB until mid-log phase (OD=0.3) at 32° C. and then washing cells three-times by using ice-cold water. Electroporated cultures were allowed to recover overnight at 37° C. and then plated to LBL agar plates containing 50 g/ml kanamycin in 145×20 mm Petri dishes (Greiner Bio-One). Plates were incubated at 37° C. until colony formation. Approximately 1000-5000 colonies were then washed off from selection plates and plasmids were extracted by using the Monarch® Plasmid Miniprep Kit (New England Biolabs). The tRNA inserts from isolated plasmids were then amplified with primers bearing the standard Nextera Illumina Read 1 and Read 2 primer binding sites, barcoded using the IDT 10 bp UDI indices, and sequenced on an Illumina NextSeq 2000, producing 150 bp paired end reads. Demultiplexing was performed with bcl-convert (v3.9.3). Paired-end reads were then trimmed using BBDuk from BBTools (in Geneious Prime® 2022.1.1, Biomatters Ltd.), merged, and aligned to their reference sequences by using Geneious assembler, implemented in Geneious Prime® 2022.1.1, allowing maximum a single SNV within the tRNA read.

tRNA-Leu_{YGA} Library Generation and Selection

We identified leucine tRNAs that can suppress TCR codons as leucine by performing two consecutive screens with plasmid libraries expressing an anticodon loop mutagenized 65,536-member library of leuU tRNA variants and a smaller, 13-member tRNA-Leu_{YGA} expression library consisting of bacteriophage derived Leu tRNA variants, both bearing two tRNAs under the control of a strong proK promoter and a UGA and CGA anticodon. To construct a 65,536-member library of leuU tRNA library, we synthesized an expression construct consisting of a proK promoter-leuU_{UGA}-leuU_{CGA}-proK terminator sequence, in which the anticodon loop of both leuU tRNAs has been fully randomized, as an oPool library (Integrated DNA Technologies, USA) (Tables 2 and 3). Next, we amplified leuU variant by using Q5 Hot Start High-Fidelity Master mix using 5′ phosphorylated primers, and then ligated the resulted library into a plasmid backbone containing a high copy-number pUC origin-of-replication and an APH(3′)-I aminoglycoside O-phosphotransferase gene in which all 29 instances of leucine coding codons were replaced with TCR serine codons (synthesized as a gBlock dsDNA fragment by Integrated DNA Technologies, USA). The ligation was performed at a 3:1 insert-to-vector ratio and by using T4 DNA ligase (NEB) for 16 hours at 16° C. according to the manufacturer's instructions. Finally, the ligation product was purified using the GeneJet PCR purification kit (Thermo Fischer Scientific, USA).

We constructed the second, 13-member tRNA-Leu_{YGA} expression library consisting of bacteriophage derived Leu tRNA variants bearing a UGA and CGA anticodon by using the same method as for our leuU_{YGA} library. Following library generation, 100 ng from each was electroporated into freshly made electrocompetent cells of Syn61Δ3 ΔrecA (ev1) and recovered in SOB broth at 32° C. for 16 hours, 250 rpm. After recovery, the cells were plated to 2×YT agar plates containing kanamycin at 200 μg/ml concentration and selection plates were incubated at 37° C. until colony formation. Finally, plasmids from clones purified using a Monarch plasmid miniprep kit (NEB) and subjected to whole plasmid sequencing (SNPsaurus, Eugene, Oregon, US).

### Virus Resistance Analysis of Ec_Syn61Δ3-SL Cells

An exponential phase culture (OD₆₀₀=0.3) of the corresponding strain was grown in 3 ml SOB broth supplemented with 10 mM CaCl₂) and MgCl2 and 75 g/ml kanamycin at 37° C. with shaking. Cultures were then spun down and resuspended in 1 ml SOB supplemented with 10 mM CaCl2) and MgCl2 and infected with a 1:1 mixture of all 12 Syn61Δ3-lytic phage isolates from this study at an MOI of 0.1. Infected cultures were incubated at 37° C. without shaking for 10 minutes for phage attachment and then washed three times with 1 ml SOB supplemented with 10 mM CaCl2) and MgCl2 by pelleting cells at 4000×g for 3 minutes. Infected cells were then diluted into 4 ml SOB supplemented with 10 mM CaCl2) and MgCl2 and 75 g/ml kanamycin and incubated at 37° C. with shaking at 250 rpm. After 24 hours of incubation, 500 μl sample was measured out into a sterile Eppendorf tube containing 50 μl chloroform, immediately vortexed, and then placed on ice. Phage infection experiments were performed in three independent replicates. Phage titers were determined by centrifuging chloroformed cultures at 6000×g for 3 minutes and then plating 5 μl of the supernatant directly or its appropriate dilutions mixed with 300 μl MDS42 cells in LBL 0.7% top agar with 10 mM CaCl2) and MgCl2. Following 18 hours of incubation at 37° C., plaques were counted, and the number of plaques were multiplied by the dilution to calculate the phage titer of the original sample.

Phage enrichment experiments were performed by mixing 50 ml early exponential phase cultures (OD₆₀₀=0.2) of bipA-biocontained Ec_Syn61Δ3-SL carrying pLS1 and pLS2 plasmids with 10 ml environmental sample mix, containing the mixture of Sample 2-13 from our study. Infected Ec_Syn61Δ3-SL cells with the corresponding plasmid were grown overnight in SOB supplemented with 200 mM bipA, 10 mM CaCl2) and MgCl2, and 75 g/ml kanamycin at 37° C. with shaking at 250 rpm. On the next day, cells were removed by centrifugation at 4000×g for 20 minutes and the supernatant was filter sterilized by using a 0.45 m filter. Next, 5 ml of the sterilized sample was mixed again with 50 ml early exponential phase cultures (OD₆₀₀=0.2) of the corresponding strain, incubated for 20 minutes at 37° C. for phage absorption, pelleted by centrifugation at 4000×g for 15 minutes, and then resuspended in 50 ml SOB supplemented with 200 mM bipA, 10 mM CaCl2) and MgCl2, and 75 g/ml kanamycin. Infected cultures were then incubated at 37° C. with shaking at 250 rpm. Cultures were grown overnight and then sterilized by centrifugation at 4000×g for 15 minutes and filtered through a 0.45 m PVDF Steriflip™ disposable vacuum filter unit (MilliporeSigma). Finally, phage titers were determined by using MDS42 cells as above. Phage enrichment experiments were performed in two independent replicates.

### Construction of pLS Plasmids

All pLS plasmids were synthesized as gBlocks by IDT and circularized either by ligation by T4 DNA ligase, or, in the case of pSC101 and RK2 plasmid-derived variants, by isothermal assembly using the HiFi DNA Assembly Master Mix (NEB). Following assembly, purified assemblies of pLS3-5 were electroporated into Ec_Syn61Δ3-SL cells carrying pLS1. pLS1 or pLS2 were designed to express two distinct combinations of the previously identified phage tRNA-Leu_{YGA} tRNAs in antiparallel orientation to avoid repeat-mediated instability, together with a pUC origin-of-replication, the aph3Ia_{29×Leu→TCR} and aminoglycoside-(3)-N-acetyltransferase_{18×Leu→TCR} marker genes. Transformants carrying pLS1 or pLS2 were identified by transforming assemblies into Syn61Δ3(ev5) ΔrecA (ev1) and selecting for kanamycin resistance. Finally, plasmids from antibiotic resistant clones purified using a Monarch plasmid miniprep kit (NEB) and subjected to whole plasmid sequencing (SNPsaurus, Eugene, Oregon, US).

### Escape Rate Analysis of Viral Leu-tRNA_{YGAS} and pLS Plasmids

We analyzed the ability of pLS plasmids to function outside Ec_Syn61Δ3-SL cells by transforming extracted plasmids from the corresponding strains cells into E. coli K-12 MG1655. Plasmids were purified from biocontained Ec_Syn61Δ3-SL cells, carrying either pLS1 or pLS2 to express tRNA-Leu_{Y}GA, or pLS1 together with pLS3-5, by using the PureLink™ Fast Low-Endotoxin Midi Plasmid Purification Kit (Thermo Fisher Scientific, USA) according to the manufacturer's instructions. Next, we electroporated 1 g from each plasmid prep into freshly made electrocompetent cells of E. coli K-12 MG1655. Cells were made electrocompetent by diluting an overnight SOB broth culture of MG1655 1:100 into 500 ml SOB broth in a 2000 ml flask and growing cells aerobically at 32° C. with shaking at 250 rpm. At OD600=0.3, cells were cooled on ice and then pelleted by centrifugation and resuspended in 10% glycerol-in-water. Cells were washed 4-times with 10% glycerol-in-water and then resuspended in 400 μl 20% glycerol-in-water. 500-500 ng from each plasmid sample was then mixed with 40-40 μl electrocompetent cells and electroporated by using standard settings (1.8 kV, 200 Ohm, 25 F) for E. coli electroporation in a 1 mm cuvette. Electroporations were performed in three replicates. Electroporated cells were then resuspended in 1 ml SOB broth, and the 2 ml culture was allowed to recover overnight at 37° C. with shaking at 250 rpm. Finally, 500 μl from each recovery culture was plated to LBL agar plates containing antibiotics corresponding to the given pLS plasmid's resistance marker (15 g/ml gentamycin plus 50 g/ml kanamycin in the case of pLS1 and 2; 100 g/ml carbenicillin, 30 g/ml chloramphenicol for pLSxx) in 145×20 mm Petri dishes (Greiner Bio-One). Plates were incubated at 37° C. for 7 days and inspected for growth.

Cloning of Phage tRNA Operon

We analyzed the incorporated amino acid in Syn61Δ3 cells bearing the tRNA operon and its native promoter from the S13_4 phage by subcloning the genomic tRNA operon into a low-copy plasmid containing a RK2 origin-of-replication and a chloramphenicol-acetyltransferase marker gene, both recoded to contain no TCR and TAG codons. The genomic tRNA operon of S13_4 was PCR amplified using the Q5 Hot Start Master Mix (NEB) from extracted phage gDNA and purified using the GeneJet PCR purification kit (Thermo Fischer Scientific, USA). Next, 100 ng of the amplified tRNA operon was assembled into the linearized pRK2-cat backbone using the HiFi DNA Assembly Master Mix (NEB). After incubation for 60 mins at 50° C., the assembly was purified using the DNA Concentrator & Clean kit (Zymo Research, USA) and transformed into Syn61Δ3 ΔrecA (ev1) cells expressing MSKGPGKVPGAGVPGxGVPGVGKGGGT (SEQ ID NO: 10)—elastin peptide fused to sfGFP with a terminal 6×His tag (in which x denotes the analyzed codon, TCA or TCG) on a plasmid containing a kanamycin resistance gene and a pUC origin of replication. Following an overnight recovery at 32° C. cells were plated to 2×YT agar plates containing kanamycin (50 μg/ml) and chloramphenicol (22 μg/ml). Finally, plasmid sequences in outgrowing colonies were validated by whole-plasmid sequencing. Elastin_{16TCR}-sfGFP-6×HIS expression measurements has been performed as described below.

### Elastin_{16TCA}-sfGFP-6×HIS Expression Measurements

We assayed the amino acid identity of the serine tRNA_{UGA}S of MZ501075, MZ501113 phages, and tRNA_{UGA}s from lytic phage isolates of Syn61Δ3, S3_1 and S13_4, by coexpressing selected tRNAs with constitutively expressed MSKGPGKVPGAGVPGxGVPGVGKGGGT (SEQ ID NO: 10)—elastin peptide fused to sfGFP with a terminal 6×His tag (in which x denotes the analyzed codon, TCA or TCG) on a plasmid containing a kanamycin resistance gene and a pUC origin of replication. Similarly, the pRK2-S13_4 plasmid carrying the tRNA array under the control of its native promoter from S13_4, was coexpressed with the same pUC plasmids carrying no tRNA genes. As control, we utilized the same elastin-sfGFP-6×HIS expression construct in which position x has been replaced with alanine. For fluorescence and MS/MS measurements, we diluted cultures 1:100 from overnight starters grown at 32° C. in 2×YT media into 50 ml 2×YT in 300 ml shake flasks containing the corresponding antibiotics and cultivated for 48 hours at 37° C., 200 rpm aerobically. We then determined sfGFP expression levels in samples by pelleting and washing 1 ml of the culture with PBS and resuspending cell pellets in 110 μL BugBuster Protein Extraction Reagent (Millipore). Reactions were incubated for 5 minutes, spun down at 13,000× rpm for 10 minutes. The fluorescence of the BugBuster-treated supernatants and the OD₆₀₀ of the original culture were measured using the Synergy H1 Hybrid Reader (BioTek) plate reader using the bottom mode analysis with an excitation at 480 nm and emission measurement at 515 nm, with a gain set to 50. The remaining 49 mL culture has been spun down and the cell pellet was resuspended in 2 ml BugBuster Protein Extraction Reagent (Millipore) and incubated at room temperature for 5 minutes. The lysed cell mixture has been spun down at 13,000× rpm for 10 min and the supernatant was mixed in 1:1 ratio with HIS-Binding/Wash Buffer (G-Biosciences, USA) and 50 μl HisTag Dynabeads (Thermo Fischer Scientific, USA). Following an incubation period of 5 mins, the beads were separated on a magnetic rack and washed with 300 μl HIS-Binding/Wash Buffer and PBS (Phosphate Buffered Saline) three-times. After the last wash step, the bead pellets containing the bound elastin-sfGFP-6×HIS were frozen at −80° C. until MS/MS sample preparation. Protein production and purification assays were all performed in three independent replicates.

### Tandem Liquid Chromatography and Mass Spectrometry (LC/MS-MS) Analysis of Tryptic Elastin-sfGFP-6×HIS

Samples from elastin-sfGFP-6×HIS expression experiments were digested directly on HisTag Dynabeads according to the FASP digest procedure. In brief, samples were washed with 50 mM TEAB (triethylammonium bicarbonate buffer) and then rehydrated with 50 mM TEAB-trypsin solution followed by a three-hour digest at 50° C. Digested peptides were then separated from HisTag Dynabeads and concentrated by spinning and drying samples at 3.000× rpm using a SpeedVac concentrator. Samples were then solubilized in 0.1% formic acid-in-water for subsequent analysis by tandem mass spectrometry. LC-MS/MS analysis of digested samples was performed on a Lumos Tribrid Orbitrap Mass Spectrometer equipped with an Ultimate 3000 nano-HPLC (both from Thermo Fisher Scientific, USA). Peptides were separated on a 150 μm inner diameter microcapillary trapping column packed first with 2 cm of C18 Reprosil resin (5 μm, 100 Å, from Dr. Maisch GmbH, Germany) followed by a 50 cm analytical column (PharmaFluidics, Belgium). Separation was achieved through applying a gradient from 4% to 30% acetonitrile in 0.1% formic acid over 60 mins at 200 nl/min. Electrospray ionization was performed by applying a voltage of 2 kV using a custom electrode junction at the end of the microcapillary column and sprayed from metal tips (PepSep, Denmark). The mass spectrometry survey scan was performed in the Orbitrap in the range of 400-1,800 m/z at a resolution of 6×10⁴, followed by the selection of the twenty most intense ions for fragmentation using Collision Induced Dissociation in the second MS step (CID-MS2 fragmentation) in the Ion trap using a precursor isolation width window of 2 m/z, AGC (automatic gain control) setting of 10,000 and a maximum ion accumulation of 100 ms. Singly charged ion species were excluded from CID fragmentation. Normalized collision energy was set to 35 V and an activation time of 10 ms. Ions in a 10-ppm m/z window around ions selected for MS-MS were excluded from further selection for fragmentation for 60 seconds.

The raw data was analyzed using Proteome Discoverer 2.4 (Thermo Fisher Scientific, USA). Assignment of MS/MS spectra was performed using the Sequest HT algorithm by searching the data against a protein sequence database including all protein entries from E. coli K-12 MG1655, all proteins sequences of interest (including the elastin-sfGFP fusion protein), as well as other known contaminants such as human keratins and common lab contaminants. Quantitative analysis between samples were performed by LFQ (label free quantitation) between different samples. Sequest HT searches were performed using a 10-ppm precursor ion tolerance and requiring each peptides N-/C termini to adhere with trypsin protease specificity, while allowing up to two missed cleavages. Methionine oxidation (+15.99492 Da), deamidation (+0.98402 Da) of asparagine and glutamine amino acids, phosphorylation at serine, threonine, and tyrosine amino acids (+79.96633 Da) and N-terminus acetylation (+42.01057 Da) was set as variable modifications. We then determined the amino acid incorporated at position X in our elastin-sfGFP construct by analyzing changes compared to Phe. To cover all 20 possible amino acids exchange cases at the X position, we performed five separate searches with four different amino acids as possible variable modification in each search. All cysteines were set to permanent no modification due to no alkylation procedure. An overall false discovery rate of 1% on both protein and peptide level was achieved by performing target-decoy database search using Percolator.

### Total Proteome Analysis and the Detection of Serine-to-Leucine Mistranslation Events

Samples from control and phage infected Syn61Δ3 cells were digested according to the FASP digest procedure. In brief, samples were washed with 50 mM TEAB buffer on a 10 kDa cutoff filter (Pall Corp, CA) and then rehydrated with 50 mM TEAB-trypsin solution followed by a three-hour digest at 37° C. Digested peptides were then extracted and separated into 10 fractions by using the Pierce™ High pH Reversed-Phase Peptide Fractionation Kit according to the manufacturer's protocol (Thermo Fisher Scientific, USA). Following fractionation, peptides were concentrated and dried by spinning samples at 3.000× rpm using a SpeedVac concentrator. Samples were then solubilized in 0.1% formic acid-in-water for subsequent analysis by tandem mass spectrometry. LC-MS/MS analysis of digested samples was performed on a Lumos Tribrid Orbitrap Mass Spectrometer equipped with an Ultimate 3000 nano-HPLC (both from Thermo Fisher Scientific, USA). Peptides were separated on a 150 μm inner diameter microcapillary trapping column packed first with 2 cm of C18 Reprosil resin (5 μm, 100 Å, from Dr. Maisch GmbH, Germany) followed by a 50 cm analytical column (PharmaFluidics, Belgium). Separation was achieved through applying a gradient from 5% to 27% acetonitrile in 0.1% formic acid over 90 mins at 200 nl/min. Electrospray ionization was performed by applying a voltage of 2 kV using a custom electrode junction at the end of the microcapillary column and sprayed from metal tips (PepSep, Denmark). The mass spectrometry survey scan was performed in the Orbitrap in the range of 400-1,800 m/z at a resolution of 6×10⁴, followed by the selection of the twenty most intense ions for fragmentation using Collision Induced Dissociation in the second MS step (CID-MS2 fragmentation) in the Ion trap using a precursor isolation width window of 2 m/z, AGC (automatic gain control) setting of 10,000 and a maximum ion accumulation of 100 ms. Singly charged ion species were excluded from CID fragmentation. Normalized collision energy was set to 35 V and an activation time of 10 ms. Ions in a 10-ppm m/z window around ions selected for MS-MS were excluded from further selection for fragmentation for 60 seconds.

The raw data was analyzed using Proteome Discoverer 2.4 (Thermo Fisher Scientific, USA). Assignment of MS/MS spectra was performed using the Sequest HT algorithm by searching the data against a protein sequence database including all protein entries from E. coli K-12 MG1655, all proteins sequences of interest (including the elastin-sfGFP fusion protein), as well as other known contaminants such as human keratins and common lab contaminants. Quantitative analysis between samples were performed by LFQ (label free quantitation) between different samples. Sequest HT searches were performed using a 10-ppm precursor ion tolerance and requiring each peptides N-/C termini to adhere with trypsin protease specificity, while allowing up to two missed cleavages. Methionine oxidation (+15.99492 Da), deamidation (+0.98402 Da) of asparagine and glutamine amino acids, phosphorylation at serine, threonine, and tyrosine amino acids (+79.96633 Da) and N-terminus acetylation (+42.01057 Da) was set as variable modifications. Special modification of serine to leucine amino acids exchange (+26.052036 Da) on all serine amino acid positions was used as variable modification. All cysteines were set to permanent no modification due to no alkylation procedure. An overall false discovery rate of 1% on both protein and peptide level was achieved by performing target-decoy database search using Percolator.

TABLE 2
Leucine tRNAs that can suppress TCR codons as leucine

| tRNA ID | Sequence |
|---|---|
| leuU_{UGA} variant 1 | GCCGAGGTGGTGGAATTGGTAGACACGCTACCTTTGAAAGGTAGTGCCCAATA |
| | GGGCTTACGGGTTCAAGTCCCGTCCTCGGTACCA (SEQ ID NO: 11) |
| leuU_{UGA} variant 2 | GCCGAGGTGGTGGAATTGGTAGACACGCTACCGTTGAATGGTAGTGCCCAATA |
| | GGGCTTACGGGTTCAAGTCCCGTCCTCGGTACCA (SEQ ID NO: 12) |
| leuU_{CGA} Variant 1 | GCCGAGGTGGTGGAATTGGTAGACACGCTACCCCCCGAAAGGTAGTGCCCAAT |
| | AGGGCTTACGGGTTCAAGTCCTTACCCTCGGTACCA (SEQ ID NO: 13) |
| leuU_{CGA} variant 2 | GCCGAGGTGGTGGAATTGGTAGACACGCTACCTTCGAATGGTAGTGCCCAATA |
| | GGGCTTACGGGTTCAAGTCCCGTCCTCGGTACCA (SEQ ID NO: 14) |
| Viral Leu-tRNA_{YGA} 1 | GCGTGATTGATGGAATTGGCATACATACCGTCCTCGAAAGTCGGGTTTTGAGG |
| (MF402939.1_tRNA9_Leu_ | GTTCGAATCCCTTGTCACGCACCA (SEQ ID NO: 1) |
| CGA) | |
| Viral Leu-tRNA_{YGA} 1 | GCGTGATTGATGGAATTGGCATACATACCGTCCTTGAAAGTCGGGTTTTGAGG |
| (MF402939.1_tRNA9_Leu_ | GTTCGAATCCCTTGTCACGCACCA (SEQ ID NO: 2) |
| UGA) | |
| Viral Leu-tRNA_{YGA} 2 | GGGGGTGTAATCGAATTGGCATAGGTACTGGACTCGAAATTCAGGTTTTGTGG |
| (MF402939.1_tRNA21_Leu_ | GTTCGAATCCCACCACCCCTACCA (SEQ ID NO: 3) |
| CGA) | |
| Viral Leu-tRNA_{YGA} 2 | GGGGGTGTAATCGAATTGGCATAGGTACTGGACTTGAAATTCAGGTTTTGTGG |
| (MF402939.1_tRNA21_Leu_ | GTTCGAATCCCACCACCCCTACCA (SEQ ID NO: 4) |
| UGA) | |
| Viral Leu-tRNA_{YGA} 3 | GCACCTATAGCCCAACTGGTAGAGGCAGCAGACTCGAAATCTGCTCAGTGTGA |
| (KP869110.1_tRNA24_Leu_ | GTTCGAATCTCTCTAGGTGTACCA (SEQ ID NO: 5) |
| CGA) | |
| Viral Leu-tRNA_{YGA} 3 | GCACCTATAGCCCAACTGGTAGAGGCAGCAGACTTGAAATCTGCTCAGTGTGA |
| (KP869110.1_tRNA24_Leu_ | GTTCGAATCTCTCTAGGTGTACCA (SEQ ID NO: 6) |
| UGA) | |

TABLE 3
Viral Genomic tRNAs

| | | | |
|---|---|---|---|
| | tRNA | Anti- | |
| Host | type | codon | tRNA sequence (5′-3′) |

Vertebrate viral genomic tRNAs

| | | | |
|---|---|---|---|
| AF105037.1 | Gly | ACC | GCCAGAGTAGCTCAATTGGTAGAGCAACAGGTCACCGATCCTGG |
| | | | TGGTTCTCGGTTCAAGTCCGAGCTCTGGTC (SEQ ID NO: 15) |
| AF105037.1 | Gln | CTG | GCCAGGGTAGCTCAATCGGTAGAGCAGCGGTTCCTGGAGTCCGC |
| | | | TGGTTCTCGGTTCAAGCCCGAGCCCTGGTT (SEQ ID NO: 16) |
| AF105037.1 | Val | AAC | GTCGGGGTAGCTCAAATGGTAGAGTGGCAGGCCAACATAGCCAG |
| | | | CAGATCTCGGTTCAAACCCGAGCCCTGACC (SEQ ID NO: 17) |
| AF105037.1 | Met | CAT | GTCGGGGTAGCTCAATTGGTAGAGCGGCAGGCTCATCCCCTGCA |
| | | | GGTTCTCGGTTCAATCCCGGGTCCCGACG (SEQ ID NO: 18) |
| AF105037.1 | Thr | AGT | GCCAGGGTAGCTCAATTGGTAGAGCATCAGGCTAGTATCCTGTC |
| | | | GGTTCCGGTTCAAGTCCGGGCCCTGGTT (SEQ ID NO: 19) |
| AF105037.1 | Undet | NNN | GCCAGCGTAGCTCAATTGTTAGAGCAGCGGCCACCAAGCCTGCA |
| | | | GGTTCTCGGTTCAAGTCCGGGCGCTGGCA (SEQ ID NO: 20) |
| AF105037.1 | Undet | NNN | GCCAGGGTAGCTCAATTGGTAGAGCGGCAGACACCACCTACGTG |
| | | | GTCTAGTCTGTGGATCTCGGTTCAAGTCCGAGTCCTGGCC |
| | | | (SEQ ID NO: 21) |
| AF105037.1 | Undet | NNN | ACCAGAGTGGCTCACCTGGTAGAGCACCAGGCTGCCCATCCTGT |
| | | | TGGTTCTCGGTTCAAATCCGAGCTCTGGTG (SEQ ID NO: 22) |
| AY666015.1 | Ile | TAT | GCTCCAGTAGCGCAATCGGCAGCGCGTGGTACTTATAATGCCAA |
| | | | GGTTGTGAGTTCGATCCTCACCTGGAGCA (SEQ ID NO: 23) |
| HM133903.1 | Cys | GCA | TCCCTTATAGCTCAGTTGGTAGAGACTTTGCCTGCAGTGCAGGA |
| | | | GACCCAGGTTCGATTCCTGGGTTGGGAA (SEQ ID NO: 24) |
| HM133903.1 | Cys | GCA | TTCCATGGTGGCTCAAAGGTTAAAGCATCTGCCTGCAATGCGGG |
| | | | AGACCCATGTTCAATCCCTGAGTGAGGAAG (SEQ ID NO: 25) |
| KF286430.1 | Asp | ATC | GGTGGTGTAGCTTAGTTGTTAGAGCGTCGGTGCATCACACCGGA |
| | | | GACCCCAGATCGAAACTGGGCATCACCA (SEQ ID NO: 26) |
| KJ452170.1 | Asp | ATC | GGTGGTGTAGCTTAGTTGTTAGAGCGTCGGTGCATCACACCGGA |
| | | | GACCCCAGATCGAAACTGGGCATCACCA (SEQ ID NO: 26) |
| KJ452171.1 | Asp | ATC | GGTGGTGTAGCTTAGTTGTTAGAGCGTCGGTGCATCACACCGGA |
| | | | GACCCCAGATCGAAACTGGGCATCACCA (SEQ ID NO: 26) |
| KJ452172.1 | Asp | ATC | GGTGGTGTAGCTTAGTTGTTAGAGCGTCGGTGCATCACACCGGA |
| | | | GACCCCAGATCGAAACTGGGCATCACCA (SEQ ID NO: 26) |
| KJ452173.1 | Asp | ATC | GGTGGTGTAGCTTAGTTGTTAGAGCGTCGGTGCATCACACCGGA |
| | | | GACCCCAGATCGAAACTGGGCATCACCA (SEQ ID NO: 26) |
| KX247011.1 | Ala | TGC | GACAGTGTAGCTCAGCCGTAAGAGCGTCGGACTTGCACTCCGGA |
| | | | GGTCCCCAGATCGAAACTGGGTACTGCCG (SEQ ID NO: 27) |
| KX247012.1 | Ala | TGC | GACAGTGTAGCTCAGCCGTAAGAGCGTCGGACTTGCACTCCGGA |
| | | | GGTCCCCAGATCGAAACTGGGTACTGCCG (SEQ ID NO: 27) |
| M12349.1 | Lys | CTT | GCCAAGACATCATCCAGTCGGTAGAGCATGGGACTCTTAATCCC |
| | | | AGGGTCGTGGGTTCGTGCCCCACGTTGGGCG (SEQ ID NO: 28) |
| MF168407.1 | Ala | TGC | GACAGTGTAGCTCAGCCGTAAGAGCGTCGGACTTGCACTCCGGA |
| | | | GGTCCCCAGATCGAAACTGGGTACTGCCG (SEQ ID NO: 27) |
| MK050972.1 | Ala | TGC | GACAGTGTAGCTCAGCCGTAAGAGCGTCGGACTTGCACTCCGGA |
| | | | GGTCCCCAGATCGAAACTGGGTACTGCCG (SEQ ID NO: 27) |
| NC_001550.1 | Lys | CTT | GCCAAGACATCATCCAGTCGGTAGAGCATGGGACTCTTAATCCC |
| | | | AGGGTCGTGGGTTCGTGCCCCACGTTGGGCG (SEQ ID NO: 28) |
| NC_001720.1 | Ala | TGC | GACAGTGTAGCTCAGCCGTAAGAGCGTCGGACTTGCACTCCGGA |
| | | | GGTCCCCAGATCGAAACTGGGTACTGCCG (SEQ ID NO: 27) |
| NC_001813.1 | Asp | ATC | GGTGGTGTAGCTTAGTTGTTAGAGCGTCGGTGCATCACACCGGA |
| | | | GACCCCAGATCGAAACTGGGCATCACCA (SEQ ID NO: 26) |
| NC_001826.2 | Gly | ACC | GCCAGAGTAGCTCAATTGGTAGAGCAACAGGTCACCGATCCTGG |
| | | | TGGTTCTCGGTTCAAGTCCGAGCTCTGGTC (SEQ ID NO: 15) |
| NC_001826.2 | Gln | CTG | GCCAGGGTAGCTCAATCGGTAGAGCAGCGGTTCCTGGAGTCCGC |
| | | | TGGTTCTCGGTTCAAGCCCGAGCCCTGGTT (SEQ ID NO: 16) |
| NC_001826.2 | Val | AAC | GTCGGGGTAGCTCAAATGGTAGAGTGGCAGGCCAACATAGCCAG |
| | | | CAGATCTCGGTTCAAACCCGAGCCCTGACC (SEQ ID NO: 17) |
| NC_001826.2 | Met | CAT | GTCGGGGTAGCTCAATTGGTAGAGCGGCAGGCTCATCCCCTGCA |
| | | | GGTTCTCGGTTCAATCCCGGGTCCCGACG (SEQ ID NO: 18) |
| NC_001826.2 | Thr | AGT | GCCAGGGTAGCTCAATTGGTAGAGCATCAGGCTAGTATCCTGTC |
| | | | GGTTCCGGTTCAAGTCCGGGCCCTGGTT (SEQ ID NO: 19) |
| NC_001826.2 | Undet | NNN | GCCAGCGTAGCTCAATTGTTAGAGCAGCGGCCACCAAGCCTGCA |
| | | | GGTTCTCGGTTCAAGTCCGGGCGCTGGCA (SEQ ID NO: 20) |
| NC_001826.2 | Undet | NNN | GCCAGGGTAGCTCAATTGGTAGAGCGGCAGACACCACCTACGTG |
| | | | GTCTAGTCTGTGGATCTCGGTTCAAGTCCGAGTCCTGGCC |
| | | | (SEQ ID NO: 21) |
| NC_001826.2 | Undet | NNN | ACCAGAGTGGCTCACCTGGTAGAGCACCAGGCTGCCCATCCTGT |
| | | | TGGTTCTCGGTTCAAATCCGAGCTCTGGTG (SEQ ID NO: 22) |
| NC_006549.1 | Ile | TAT | GCTCCAGTAGCGCAATCGGCAGCGCGTGGTACTTATAATGCCAA |
| | | | GGTTGTGAGTTCGATCCTCACCTGGAGCA (SEQ ID NO: 23) |
| NC_014564.2 | Pro | CGG | GGCGGTGTAGCTCAACGGCAGAGCGCCGGCCCCGTCCCCGCGCT |
| | | | CCCCGGGTTCGGGACCGGAGGGACCGAGATCGACCCTCGGCATC |
| | | | GCCG (SEQ ID NO: 29) |
| NC_021221.1 | Leu | TAA | GGCGGTATAGCTCAGCTGTCAGAGCGTCGGACCTAAAATCCGGA |
| | | | GGACCCCAGATCAAAACTGGGTATCGCCG (SEQ ID NO: 30) |
| NC_022612.1 | Val | AAC | GGCGGCATAGCTCAGGTGGCAGAGCGTCGGACTAACAGTTCGGA |
| | | | GGTCCCCAGATCGAAACTGGGTGCAGTCG (SEQ ID NO: 31) |
| NC_022613.1 | Phe | AAA | GGCGGTGTAGCTCAGCCGTAAGAGCGTCGGGTCAAAAGTCCGGA |
| | | | GGTCCCCAGATCGAAACTGGGTATCGCCG (SEQ ID NO: 32) |
| NC_024450.1 | Arg | CCT | TCCCTCGTTAGTTCAGTGGTAGAATACCCGCCTCCTCTCCGGGC |
| | | | GGGAGACCGAGGTTCGATTCCTCGACGAGGAA (SEQ ID NO: |
| | | | 33) |
| NC_024450.1 | Ala | CGC | GTCCCGATAGTACAACGGCTAGTATGCCCGCACCGCCCGGATGC |
| | | | CGCTCTGGCGGCGGCGGCCGTGCGGGGGACCGGGGTTCGACTCC |
| | | | CCGACGGGACG (SEQ ID NO: 34) |
| NC_024450.1 | Arg | CCG | TCCTCGTTAGTATAGCGGCTAGTACGCCCGCGGCCGACCCGGCA |
| | | | TACAGATACCGGCCGCGGGAGACCGGGGTTCGAGTCCCCGACGA |
| | | | GGGT (SEQ ID NO: 35) |
| NC_024450.1 | Cys | ACA | TCCTCGTTAGTATAGCGGTTAGTACGCCCGCAACACTAACCCGC |
| | | | CCCCATTTGCACAAGCGGGAGACCGGGGTTCGAATCCCCGACGA |
| | | | GGAA (SEQ ID NO: 36) |
| NC_024450.1 | Arg | TCT | TCCTCGTTAGTTTAGAGGTAGAACGCCCATCGTCTTCGCGAGGA |
| | | | GAATGGGAGACCGGGGTTCGAGTCCCCGACGGGGAT (SEQ ID |
| | | | NO: 37) |
| NC_024450.1 | Asp | GTC | TCCTCGTTAGTATAGTGGTTAGTATTCCCGTCTGTCGCACGGGA |
| | | | GACCGGGGTTCGATTCCCCGACGAGGAG (SEQ ID NO: 38) |
| NC_024450.1 | Leu | GAG | GTCCCCGTTTGTACATTGGTAGTACTCCCGCTCGAGGTGCTGGG |
| | | | ATTGCGGGGTTCGAGTCCCCGGCGGGGACA (SEQ ID NO: 39) |
| NC_024450.1 | Ala | CGC | TCCTCGTTAGTATAGTGGTAGTACAACCGGTTCGCGCGGGCGGC |
| | | | CGGGAGACCGAGGTTCGATTCCTCGGCGAGGAC (SEQ ID NO: |
| | | | 40) |
| NC_024450.1 | Val | CAC | TCCCCGTTAGTTTAGTGGTATAACACCCATCTCACCTTGGTAGA |
| | | | CGGGAGACCGGGGTTCGATTCCCCGACGGGGAC (SEQ ID NO: |
| | | | 41) |
| NC_024450.1 | Leu | CAA | TCCTCGTTAGTATAGTGGCAGAACACCCATCTCAAGGCCTCCGG |
| | | | ATGGGAGACCGGGGTTCGATTCCCCGACGAGGGT (SEQ ID NO: |
| | | | 42) |
| NC_024450.1 | Val | AAC | TCCTCGTTAGTACAGTGGCAGTACGCAGCCTGTCAACGCAGACG |
| | | | GGCAAACCGGGGTTCGATTCCCCGACGAGGAT (SEQ ID NO: |
| | | | 43) |
| NC_024450.1 | Tyr | ATA | TCCTCGGTAGTACAACGGTTACTACGCCCGTATATATATAGAGT |
| | | | AAACGGGAGATCGGGGTTCGACTCCCCGCCGAGGAA (SEQ ID |
| | | | NO: 44) |
| NC_024450.1 | Asp | GTC | TCCTCGTTAGTATAGTGGTAGTATGCCCGTCTGTCGCACGGGAG |
| | | | ACCGGGGTTCGATTCCCCGACGAGGAG (SEQ ID NO: 45) |
| NC_024450.1 | Arg | CCT | TCCCTCGTTAGTTCAGTGGTAGAATACCCGCCTCCTCTCCGGGC |
| | | | GGGAGACCGAGGTTCGATTCCTCGACGAGGAA (SEQ ID NO: |
| | | | 33) |
| NC_024450.1 | Ala | CGC | GTCCCGATAGTACAACGGCTAGTATGCCCGCACCGCCCGGATGC |
| | | | CGCTCTGGCGGCGGCGGCCGTGCGGGGGACCGGGGTTCGACTCC |
| | | | CCGACGGGACG (SEQ ID NO: 34) |
| NC_024450.1 | Arg | CCG | TCCTCGTTAGTATAGCGGCTAGTACGCCCGCGGCCGACCCGGCA |
| | | | TACAGATACCGGCCGCGGGAGACCGGGGTTCGAGTCCCCGACGA |
| | | | GGGT (SEQ ID NO: 35) |
| NC_024450.1 | Ser | GGA | CCCTCGTTAGTCTAGCGGCAAAACGCCCAGTCTGGAATGTCGAT |
| | | | GGGAGTCTGGGGTTCGATTCCCCAACGAGGGT (SEQ ID NO: |
| | | | 46) |
| NC_024450.1 | Undet | NNN | CCCTCATTAGTACAGCGGTTAGTACACCCGGACGCTCCACGGAC |
| | | | ACACGGGAGAGCGGGGTTCGACTCCCCGATGAGGGC (SEQ ID |
| | | | NO: 47) |
| NC_024450.1 | Ser | TGA | TCCTCGTTAGTACAGCGGTTAGTATACGCACTTGACTGTTTGTC |
| | | | AGGTAGCGAGAGCGGGGTTCGACTCCCCGGCGGGGAT (SEQ ID |
| | | | NO: 48) |
| NC_024450.1 | Thr | GGT | GCCTCGCTAGTACAGGGGTTAGTATGCCCGTATGGTATATGTGT |
| | | | CACGCGGGAGACCGGGGTTCGACTCCCCGGCGAGGCG (SEQ ID |
| | | | NO: 49) |
| NC_024450.1 | Val | AAC | CCCTTGTTAGTACAGCGGTTAGTATACCAGTACGGCCCAACAAC |
| | | | CGTGCTGGAGACCGGGGTTCGACTCCCCGACGAGGGT (SEQ ID |
| | | | NO: 50) |
| NC_024450.1 | Ser | GGA | TCCCCGTTAGTACAGTGGCAGTACGCCGTCCGTGGAGCGGATGG |
| | | | ACAACCGGGGTTCGATTCCCCGACGGGGCA (SEQ ID NO: 51) |
| NC_024450.1 | Sup | TTA | TCCTCGTTAGTATAGCGGTTAATACACCCGCCGAAAGGCTTTAG |
| | | | ACCTTTGACGGGAAAGTGGGGTTCGACTCCCCGACGAGGAT |
| | | | (SEQ ID NO: 52) |
| NC_024450.1 | Ile | AAT | TCCTCGTTAGTACAGCGGTTAGTACGCCAACCCAATAGCTATCT |
| | | | GGGTTGGAAACCGGGGTTCAACTCCCCGACGAGGAT (SEQ ID |
| | | | NO: 53) |
| NC_024450.1 | His | GTG | CCCTCGCTAGTATAGTGGTTATTACGCTCGGCGGTGCCTAGGCC |
| | | | GAGAGACCGGGGTTCGATCCCCCGGCGAGGGA (SEQ ID NO: |
| | | | 54) |
| NC_024450.1 | Leu | GAG | TCCCCATTAGTACAGCGGTTAGTACACTCGCATGAGTCATACCT |
| | | | GATGCGAGAGACCGGGGTTCGACTCCCCGATGGGGGA (SEQ ID |
| | | | NO: 55) |
| NC_024450.1 | Asp | GTC | TCCTCGTTAGTATAGTGGTTAATATCCCCGTTTGTCGCACGGGA |
| | | | GACCGGGGTTCGAATCCCCGACGAGGAG (SEQ ID NO: 56) |
| NC_026141.2 | Phe | GAA | GCTGAAGTAGCTCAGTTGGGAGAGCATTAGACTGAAGATCTAAA |
| | | | GGTCCCTGGTTCAACCCCGGGCTTCAGCA (SEQ ID NO: 57) |
| NC_027778.1 | Ile | TAT | GCTCTAGTGGCGCAGTTAGGTAGCGCGCAGTGCTTATAACACTG |
| | | | AGGTCGTGAGTTCGATCCTCACCTAGAGCA (SEQ ID NO: 58) |
| NC_034266.1 | Arg | CCT | TCCCTCGTTAGTTCAGTGGTAGAATACCCGCCTCCTCTCCGGGC |
| | | | GGGAGACCGAGGTTCGATTCCTCGACGAGGAA (SEQ ID NO: |
| | | | 33) |
| NC_034266.1 | Ala | CGC | GTCCCGATAGTACAACGGCTAGTATGCCCGCACCGCCCGGATGC |
| | | | CGCTCTGGCGGCGGCGGCCGTGCGGGGGACCGGGGTTCGACTCC |
| | | | CCGACGGGACG (SEQ ID NO: 34) |
| NC_034266.1 | Arg | CCG | TCCTCGTTAGTATAGCGGCTAGTACGCCCGCGGCCGACCCGGCA |
| | | | TACAGATACCGGCCGCGGGAGACCGGGGTTCGAGTCCCCGACGA |
| | | | GGGT (SEQ ID NO: 35) |
| NC_034266.1 | Cys | ACA | TCCTCGTTAGTATAGCGGTTAGTACGCCCGCAACACTAACCCGC |
| | | | CCCCATTTGCACAAGCGGGAGACCGGGGTTCGAATCCCCGACGA |
| | | | GGAA (SEQ ID NO: 36) |
| NC_034266.1 | Arg | TCT | TCCTCGTTAGTTTAGAGGTAGAACGCCCATCGTCTTCGCGAGGA |
| | | | GAATGGGAGACCGGGGTTCGAGTCCCCGACGGGGAT (SEQ ID |
| | | | NO: 37) |
| NC_034266.1 | Asp | GTC | TCCTCGTTAGTATAGTGGTTAGTATTCCCGTCTGTCGCACGGGA |
| | | | GACCGGGGTTCGATTCCCCGACGAGGAG (SEQ ID NO: 38) |
| NC_034266.1 | Leu | GAG | GTCCCCGTTTGTACATTGGTAGTACTCCCGCTCGAGGTGCTGGG |
| | | | ATTGCGGGGTTCGAGTCCCCGGCGGGGACA (SEQ ID NO: 39) |
| NC_034266.1 | Ala | CGC | TCCTCGTTAGTATAGTGGTAGTACAACCGGTTCGCGCGGGCGGC |
| | | | CGGGAGACCGAGGTTCGATTCCTCGGCGAGGAC (SEQ ID NO: |
| | | | 40) |
| NC_034266.1 | Val | CAC | TCCCCGTTAGTTTAGTGGTATAACACCCATCTCACCTTGGTAGA |
| | | | CGGGAGACCGGGGTTCGATTCCCCGACGGGGAC (SEQ ID NO: |
| | | | 41) |
| NC_034266.1 | Leu | CAA | TCCTCGTTAGTATAGTGGCAGAACACCCATCTCAAGGCCTCCGG |
| | | | ATGGGAGACCGGGGTTCGATTCCCCGACGAGGGT (SEQ ID NO: |
| | | | 42) |
| NC_034266.1 | Val | AAC | TCCTCGTTAGTACAGTGGCAGTACGCAGCCTGTCAACGCAGACG |
| | | | GGCAAACCGGGGTTCGATTCCCCGACGAGGAT (SEQ ID NO: |
| | | | 43) |
| NC_034266.1 | Tyr | ATA | TCCTCGGTAGTACAACGGTTACTACGCCCGTATATATATATAGA |
| | | | GTAAACGGGAGATCGGGGTTCGACTCCCCGCCGAGGAA (SEQ |
| | | | ID NO: 59) |
| NC_034266.1 | Asp | GTC | TCCTCGTTAGTATAGTGGTAGTATGCCCGTCTGTCGCACGGGAG |
| | | | ACCGGGGTTCGATTCCCCGACGAGGAG (SEQ ID NO: 45) |
| NC_034266.1 | Arg | CCT | TCCCTCGTTAGTTCAGTGGTAGAATACCCGCCTCCTCTCCGGGC |
| | | | GGGAGACCGAGGTTCGATTCCTCGACGAGGAA (SEQ ID NO: |
| | | | 33) |
| NC_034266.1 | Ala | CGC | GTCCCGATAGTACAACGGCTAGTATGCCCGCACCGCCCGGATGC |
| | | | CGCTCTGGCGGCGGCGGCCGTGCGGGGGACCGGGGTTCGACTCC |
| | | | CCGACGGGACG (SEQ ID NO: 34) |
| NC_034266.1 | Arg | CCG | TCCTCGTTAGTATAGCGGCTAGTACGCCCGCGGCCGACCCGGCA |
| | | | TACAGATACCGGCCGCGGGAGACCGGGGTTCGAGTCCCCGACGA |
| | | | GGGT (SEQ ID NO: 35) |
| NC_034266.1 | Ser | GGA | CCCTCGTTAGTCTAGCGGCAAAACGCCCAGTCTGGAATGTCGAT |
| | | | GGGAGTCTGGGGTTCGATTCCCCAACGAGGGT (SEQ ID NO: |
| | | | 46) |
| NC_034266.1 | Undet | NNN | CCCTCATTAGTACAGCGGTTAGTACACCCGGACGCTCCACGGAC |
| | | | ACACGGGAGAGCGGGGTTCGACTCCCCGATGAGGGC (SEQ ID |
| | | | NO: 47) |
| NC_034266.1 | Ser | TGA | TCCTCGTTAGTACAGCGGTTAGTATACGCACTTGACTGTTTGTC |
| | | | AGGTAGCGAGAGCGGGGTTCGACTCCCCGGCGGGGAT (SEQ ID |
| | | | NO: 48) |
| NC_034266.1 | Thr | GGT | GCCTCGCTAGTACAGGGGTTAGTATGCCCGTATGGTATATGTGT |
| | | | CACGCGGGAGACCGGGGTTCGACTCCCCGGCGAGGCG (SEQ ID |
| | | | NO: 49) |
| NC_034266.1 | Val | AAC | CCCTTGTTAGTACAGCGGTTAGTATACCAGTACGGCCCAACAAC |
| | | | CGTGCTGGAGACCGGGGTTCGACTCCCCGACGAGGGT (SEQ ID |
| | | | NO: 50) |
| NC_034266.1 | Ser | GGA | TCCCCGTTAGTACAGTGGCAGTACGCCGTCCGTGGAGCGGATGG |
| | | | ACAACCGGGGCTCGATTCCCCGACGGGGCA (SEQ ID NO: 60) |
| NC_034266.1 | Ile | AAT | TCCTCGTTAGTACAGCGGTTAGTACGCCAACCCAATAGCTATCT |
| | | | GGGTTGGAAACCGGGGTTCAACTCCCCGACGAGGAT (SEQ ID |
| | | | NO: 53) |
| NC_034266.1 | His | GTG | CCCTCGCTAGTATAGTGGTTATTACGCTCGGCGGTGCCTAGGCC |
| | | | GAGAGACCGGGGTTCGATCCCCCGGCGAGGGA (SEQ ID NO: |
| | | | 54) |
| NC_034266.1 | Leu | GAG | TCCCCATTAGTACAGCGGTTAGTACACTCGCATGAGTCATACCT |
| | | | GATGCGAGAGACCGGGGTTCGACTCCCCGATGGGGGA (SEQ ID |
| | | | NO: 55) |
| NC_034266.1 | Asp | GTC | TCCTCGTTAGTATAGTGGTTAATATCCCCGTTTGTCGCACGGGA |
| | | | GACCGGGGTTCGAATCCCCGACGAGGAG (SEQ ID NO: 56) |
| NC_038668.1 | Ala | AGC | TTTGGGGGTTGGGGATTTAGCTCAGTGATAGAGCTCTTGCCTAG |
| | | | CACGCAAGCCCTGGGTTCGGTCCCCCCAGCTCTGAAA (SEQ ID |
| | | | NO: 61) |
| NC_040671.1 | Tyr | GTA | GGCCAGATACCGTAGGGGTTACACAACCAGGTTGTAACACTTGG |
| | | | GTGTCTGGGTTCGATTTCCAGTCTGGCCG (SEQ ID NO: 62) |
| NC_040681.1 | Phe | GAA | GCCGAGATAGCTCAGTAGGGAGAGCGTCGGACTGAAGTTCTGAA |
| | | | GGTCCCTGGTTCGATCCCAGGTTTCGGCA (SEQ ID NO: 63) |
| NC_055135.1 | Glu | TTC | ATCTATGTTGGCTTAGTGGTTACTAGGCAGGGAGATAAGCGAGG |
| | | | TTTTCAAGCCACCTACTCCTGGGTTCGAACCCCAGCATAGATT |
| | | | (SEQ ID NO: 64) |
| NC_055233.1 | Gly | ACC | GCCAGAGTAGCTCAATTGGTAGAGCAACAGGTCACCGATCCTGG |
| | | | TGGTTCTCGGTTCAAGTCCGAGCTCTGGTC (SEQ ID NO: 15) |
| NC_055233.1 | Gln | CTG | GCCAGGGTAGCTCAATCGGTAGAGTAGCGGTCCCTGGAGTCCGC |
| | | | TGGTTCTCGGTTCAAGCCCGAGCCCTGGTT (SEQ ID NO: 65) |
| NC_055233.1 | Val | AAC | GTCGGGGTAGCTCAAATGGTAGAGTGGCAGGCCAACATAGCCAG |
| | | | CAGATCTCGGTTCAAACCCGAGCTCTGACC (SEQ ID NO: 66) |
| NC_055233.1 | Met | CAT | GTCGGGGTAGCTCAATTGGTAGAGCGGCAGGCTCATCCCCTGCA |
| | | | GGTTCTCGGTTCGATCCCGGGTCCCGGCG (SEQ ID NO: 67) |
| NC_055233.1 | Thr | AGT | GCCAGGGTAGCTCAAGTGGTAGAGCATCAGGCTAGTACCCTGTC |
| | | | GGTTCCGGTTCAAGTCCGGGCCCTGGTT (SEQ ID NO: 68) |
| NC_055233.1 | Undet | NNN | GCCAGCGTAGCTCAATTGTTAGAGCAGCGGCCACCAAGCCTGCA |
| | | | GGTTCTCGGTTCAAGTCCGGGCGCTGGCA (SEQ ID NO: 20) |
| NC_055233.1 | Undet | NNN | GCCAGGGTAGCTCAATTGGTAGAGCGACAGCCACCACCTACGTG |
| | | | GTCTAGTCTGTGGATCTCGGTTCAAGTCCGAGTCCTGGCC |
| | | | (SEQ ID NO: 69) |
| NC_055233.1 | Undet | NNN | ACCAGAGTGGCTCACCTGGTAGAGCACCAGGCTGCCCATCCTGT |
| | | | TGGTTCTCGGTTCAAGTCCCAGCTCTGGTG (SEQ ID NO: 70) |

Archaeal viral genomic tRNAs

| | | | |
|---|---|---|---|
| Acidianus_filamentous_ | Lys | CTT | GGGCTCGTAGCTTAGCCAGGTAGAGCAACGGGCTCTTAACCTAG |
| virus_2_-_ | | | GGAGATACCCGTAGGTCCCGGGTTCGAATCCCGGCGAGCCCG |
| NC_009884.1 | | | (SEQ ID NO: 71) |
| Acidianus_spindle- | Ser | CGA | TGCGTAAAATGGCAACTTCTTTCAATTCTTCAGCACGATCACGT |
| shaped_virus_1_-_ | | | AGCTCATCCAGTTCACGGCGGGCTTTTAACCCGTAGGTCCCGGG |
| NC_013585.1 | | | TTCGAATCCCGGCGGGCCCGCCA (SEQ ID NO: 72) |
| Aeropyrum_globular_ | Glu | TTC | ACGGTAGGCTGGGCGCCCCGCCTACTGCGGGCCTTTCGAGCCCG |
| virus_1_-_ | | | TGACCCGGGTTCAAATCCCGGCCGCGGCACCA (SEQ ID NO: |
| NC_036595.1 | | | 73) |
| Aeropyrum_pernix_ | Undet | NNN | GGGGGCGGGCTTAAGACCCGTTGGCGTAGGCCTGCGTGGGTTCA |
| ovoid_virus_1_-_ | | | AATCCCACCCCCCGCACCA (SEQ ID NO: 74) |
| NC_028256.1 | | | |
| Aeropyrum_pernix_ | Leu | CAA | GGTGTAGGTTATGAGAGTGGTTGAGAGGCTTGGAGCTCCACACA |
| spindle- | | | GCTATACGCCTGCTAAACACGCCTACACCGGGCCCCGCCCTTAC |
| shaped_virus_1_-_ | | | ACGGCGGAGGTCCGGGGTTCAAATCCCCGCGGGCCCACCA |
| NC_028268.1 | | | (SEQ ID NO: 75) |
| Haloarcula_hispanica_ | Arg | TCG | GGGCAGGTGAGTCGAGCGGCCGAAACTCCCGGGACTCCGGGGTA |
| icosahedral_virus_2_-_ | | | ATGCGGCGAGGCGACCTAGGCGCTGTCGACCCGTACCGCGGGTT |
| NC_016989.1 | | | CAAATCCCGCCCTGCCCG (SEQ ID NO: 76) |
| Haloferax_tailed_ | Thr | CGT | GGGAAGGTAGCTCAATGGTAGAGCCAAAGCGCGTATTGAACCTC |
| virus_1_-_ | | | CCAACTACGGGGGTGACGCGACGGTGACACGTAAGTACCTCACC |
| NC_062739.1 | | | GGGGATGTTGGTTCGAATCCAACCCTTTCCG (SEQ ID NO: 77) |
| Halovirus_HCTV-1_-_ | Gln | TTG | AGTCCCCTGGGGTAGTTTGGCCAATCCTCTGGGCCTTTGGAGCT |
| NC_021330.1 | | | CAGGACGCGAGTTCAAATCTCGCGGGGACAA (SEQ ID NO: 78) |
| Halovirus_HCTV-5_-_ | Gln | TTG | AGTCCCCTGGGGTAGTTTGGCCAATCCTCAGCGCCTTTGGAGCG |
| NC_021327.1 | | | CTGGACGCGAGTTCAAATCTCGCGGGGACAA (SEQ ID NO: 79) |
| Halovirus_HGTV-1_-_ | Undet | NNN | AGCCCCGTGGCCGAATGGTTCAAGGCGCTATTACGTCGCTTCTG |
| NC_021328.1 | | | AGATTTCAGCCACGTTTTGTGGAGCGATTGGAAGCGTTACTTCG |
| | | | CTATCTGGAAGCAGAGATTGTAGGTTCGAATCCTACCGGGGCTA |
| | | | (SEQ ID NO: 80) |
| Halovirus_HGTV-1_-_ | Arg | GCG | GGGCGATTGGTCTACTGGATATGACACATCCCTGCGAAGGATGA |
| NC_021328.1 | | | GAAGGGAGTTCGATTCTCTCATTGCCCA (SEQ ID NO: 81) |
| Halovirus_HGTV-1_-_ | Asp | GTC | GCCGAAGTGGTCTAAAAGTAAGATGCGTGCCTGTCACGCACGAG |
| NC_021328.1 | | | ACTCGGGAGCATTACCCGGCTTCGGCG (SEQ ID NO: 82) |
| Halovirus_HGTV-1_-_ | Glu | TTC | GCTGGCTTGGGGTAGTGGACGCACGGCATTAAGCCAATCCTTCG |
| NC_021328.1 | | | ACCCTTTCGAGGTCGAGACACGAGTTCGAATCTCGTAGCCAGTA |
| | | | (SEQ ID NO: 83) |
| Halovirus_HGTV-1_-_ | Phe | GAA | GGGTCGATAGGATAATTTGGGAATCCACCTGTTTGAAGAACAGG |
| NC_021328.1 | | | CGCATCTCGGTTCAAATCCGGGTCGGCCCA (SEQ ID NO: 84) |
| Halovirus_HGTV-1_-_ | Glu | CTC | GCTGATGGAGTCTCCGCTGGCGAGAGACGCTACGTTCTCAGCGT |
| NC_021328.1 | | | AGAGGTCGCGGGTTCAATTCCTGTCATCAGCA (SEQ ID NO: |
| | | | 85) |
| Halovirus_HGTV-1_-_ | Gly | TCC | GCGTGGCTGTTCCAATGGCAAGATGCGGGCCTTCCAAGCCTGAG |
| NC_021328.1 | | | ATACGAGTTCGACTCTCGTGCCGCGCA (SEQ ID NO: 86) |
| Halovirus_HGTV-1_-_ | Gly | GCC | GCGTGGTTGGTCCAATGGAAAGACGGCTCCCTGCCACGGAGCAG |
| NC_021328.1 | | | ACTCGGGTTCGATTCCCGAATCGCGCA (SEQ ID NO: 87) |
| Halovirus_HGTV-1_-_ | Asn | GTT | GTCGCGGTAGTTCAATTAGTAGAACACTCGGCTGTTACCCGAGA |
| NC_021328.1 | | | TGTTGGAGGTGCAAGTCCTCCCTGCGACG (SEQ ID NO: 88) |
| Halovirus_HGTV-1_-_ | Tyr | GTA | CTCTCGAAGACCCGCTGGTAAGGGTACACGCCTGTAGAGCGTGT |
| NC_021328.1 | | | TGCAGTAGGTTCGAATCCTACTCGGGAGA (SEQ ID NO: 89) |
| Halovirus_HGTV-1_-_ | Lys | CTT | GGGCTGATGGCGAAACTTGGCTAAACGCGGAAGGCTCTTGACCT |
| NC_021328.1 | | | TCTACATACGGGTTCAAATCCTGTTCAGCCCA (SEQ ID NO: |
| | | | 90) |
| Halovirus_HGTV-1_-_ | Met | CAT | CCCGACGTTGAGCAGTCTGGCTATGCTCGGCAGGCTCATATCCT |
| NC_021328.1 | | | GTTGTGCTTCGCACGAAATGGGTTCGAATCCCATCGTCGGAA |
| | | | (SEQ ID NO: 91) |
| Halovirus_HGTV-1_-_ | Ala | TGC | GGGCCGCTGGTCTAATGGCAAGACACTTGCTTTGCAAGAGTTGA |
| NC_021328.1 | | | AGTTACGGCTTCTGCTCCAAATTAGCAAGGAACTCCCGGTTCGA |
| | | | ATCCGGGGCGGTCCA (SEQ ID NO: 92) |
| Halovirus_HGTV-1_-_ | Thr | CGT | TCCGTTGTGGCCCAATTTGGACGGGCACTCGCCTCGTAAGCGAG |
| NC_021328.1 | | | AGGTTGTGGGTTCGATTCCCACCAGCGGAT (SEQ ID NO: 93) |
| Halovirus_HGTV-1_-_ | Thr | GGT | TCCAGTATGGTCTAATGGTAGGGCACGGCTTTGGTATGGCTGAC |
| NC_021328.1 | | | GTGAGGGTTCAACTCCCTCTGCTGGAT (SEQ ID NO: 94) |
| Halovirus_HGTV-1_-_ | Lys | TTT | GGGCCGGTGGCGAAATGGCTTAACGCAGGAGGCTTTTAACCTCC |
| NC_021328.1 | | | CATCGCGGGTTCGATTCCTGCTCGGCCCA (SEQ ID NO: 95) |
| Halovirus_HGTV-1_-_ | Gln | TTG | AGTCCCCGAGTCCGCTGGCGAAAGACGCTCGGCTTTGGACCGAG |
| NC_021328.1 | | | AGAGCGAGGTTCGATTCCTCGGGGGACTA (SEQ ID NO: 96) |
| Halovirus_HGTV-1_-_ | Trp | CCA | GAGGTCTTGGGGTAATAGAATCCTACTCGCCTCCAGAGCGAGTG |
| NC_021328.1 | | | ATGGGAGTGCAAATCTCTCAGACCTCA (SEQ ID NO: 97) |
| Halovirus_HGTV-1_-_ | Ser | CGA | GCCGGTGGTTGAGCAATCTGGTCGCTCACCTGTCTCGAAAACAG |
| NC_021328.1 | | | GCTCCCATTGTGGAATGCAGGTTCGAATCCTGCCCTCGGCG |
| | | | (SEQ ID NO: 98) |
| Halovirus_HGTV-1_-_ | Ile | GAT | ACGAGGATAGTGTAATGGAAACACTCCGGTCAGATGGACCGGCA |
| NC_021328.1 | | | CTACCTGTTCGATTCAGGTTCCTCGTA (SEQ ID NO: 99) |
| Halovirus_HGTV-1_-_ | Ser | GCT | GCCGTGGTTGAGTCAGGCAAACTCGGTTGGATTGCTAATCCAAC |
| NC_021328.1 | | | GGCCTTTATGGTCAGAAGGTTCGAATCCTTCCCTCGGCG (SEQ |
| | | | ID NO: 100) |
| Halovirus_HGTV-1_-_ | Gln | CTG | AGTCGGTTTGTGTAGCGGCCAATCATACGAGGCTCTGACCCTCG |
| NC_021328.1 | | | TGACAGAGGTTCGAATCCTCTACCGACTA (SEQ ID NO: 101) |
| Halovirus_HGTV-1_-_ | Ile | GAT | GCGTTCGTAGCCAAATCAGGTAAGGCGTTCCGCTGATAACGGAA |
| NC_021328.1 | | | AGATTTGTTGGTTCAAATCCAACCGGACGCA (SEQ ID NO: |
| | | | 102) |
| Halovirus_HGTV-1_-_ | Cys | GCA | GTCGGGATGCCAGAGTGGTCACGGCCCGGACAGCAGATTCGGTG |
| NC_021328.1 | | | TACGGGGGTTCGAATCCCTCTCCCGACT (SEQ ID NO: 103) |
| Halovirus_HGTV-1_-_ | Arg | TCG | GCCCACGTAGCCTAATTGGATAAGGCAATGGCCTTCGAAGCCAG |
| NC_021328.1 | | | AGATTGCAGGTTCGAGTCCTGTCGTGGGCG (SEQ ID NO: 104) |
| Halovirus_HGTV-1_-_ | Arg | TCT | AGGCGTATAAGGTAGTGGACTATCCCGCGAGTCTTCTAAACTTG |
| NC_021328.1 | | | CGACAGGGGTTCGAATCCCCTTACGCCTG (SEQ ID NO: 105) |
| Halovirus_HGTV-1_-_ | Leu | TAG | GAGTCGGTACGAGAAATGGTTAAACTCACGAGGCTTAGGACCTC |
| NC_021328.1 | | | GCGCTTTAGTGCTTTGCAGGTTCGAATCCTGCCCGGCTCA |
| | | | (SEQ ID NO: 106) |
| Halovirus_HGTV-1_-_ | Leu | GAG | GCATCTGTAGGGAACTGGTGAAACCCATAGGCTTGAGGGGCCTT |
| NC_021328.1 | | | GCCCGTAGGGGCTTTCTTGGTTCGATTCCAAGCGGGTGCA |
| | | | (SEQ ID NO: 107) |
| Halovirus_HGTV-1_-_ | Leu | CAA | GAGTGGATAGCCAAGTGGTCAAAGGCAGGAGAGTCAAATCCTCC |
| NC_021328.1 | | | CGGTATAGTCCTTCGCAGGTTCGAATCCTGCTCCGCTCA (SEQ |
| | | | ID NO: 108) |
| Halovirus_HGTV-1_-_ | Leu | TAA | AGGTCGGTGCGCCGAGTCTGGTCAAAGGCAGGGCACTTAAGATG |
| NC_021328.1 | | | CCCCGATTAGTTCTTCACAGGTTCAAATCCTGTCCGGCCTA |
| | | | (SEQ ID NO: 109) |
| Halovirus_HGTV-1_-_ | Pro | TGG | TGGCTGGTGGTCCAATGGTACGATTCGGCGTTTGGGACGCCGAG |
| NC_021328.1 | | | ATATGGGTTCGACTCCCTTCCAGCCAA (SEQ ID NO: 110) |
| Halovirus_HGTV-1_-_ | Val | TAC | ACGGGTATAATTCAATCTGGTCAGAATACTCCGCTTACATCGGA |
| NC_021328.1 | | | GATGTTGAAGGTTCAAATCCTTCTGCCCGTA (SEQ ID NO: |
| | | | 111) |
| Halovirus_HGTV-1_-_ | Val | GAC | GGGTGTGTGGTCCAACTCGGTTAAGATACCGCATTGACATTGCG |
| NC_021328.1 | | | GTGATTTGAGGTTCAAATCCTCTCGCACCCA (SEQ ID NO: |
| | | | 112) |
| Halovirus_HVTV-1_-_ | Gln | TTG | AGTCCCCTGGGGTAGTTTGGCCAATCCTCTGGGCCTTTGGAGCC |
| NC_020158.1 | | | CAGGACGCGAGTTCAAATCTCGCGGGGACAA (SEQ ID NO: |
| | | | 113) |
| Sulfolobus_spindle- | Leu | CAA | GGCGCAAATTGGCAACCAACGCTATTTTACGTCGTTCAGTTCCC |
| shaped_virus_1_-_ | | | TCTTTTCCGCCTCCGGAGCCGGAGGTCCCGGGTTCAAATCCCGG |
| NC_001338.1 | | | CGGGTCCG (SEQ ID NO: 114) |
| Sulfolobus_spindle- | Gly | CCC | GGGGTGTCCCCCTAATGCCGGCCTCCCAAGCCGGTGATCCCGGG |
| shaped_virus_2_-_ | | | TTCAAATCCCGGCGGCCGCA (SEQ ID NO: 115) |
| NC_005265.1 | | | |
| Sulfolobus_spindle- | Glu | TTC | GCAGTGGGGGTTTTAGGGGGTGTCCCCCCTAATGCGGGCCTTTC |
| shaped_virus_4_-_ | | | GAGCCCGTGACCCGGGTTCAAATCCCGGCCGCGGCA (SEQ ID |
| NC_009986.1 | | | NO: 116) |
| Sulfolobus_spindle- | Gln | CTG | GGGGGTTGTGGGGGCGTTACCCCCACAGGGCTCTGGCCCCTGGG |
| shaped_virus_5_-_ | | | ACCAGGGTTCGAATCCCTGCCCGGCTA (SEQ ID NO: 117) |
| NC_011217.1 | | | |
| Sulfolobus_spindle- | Gln | CTG | AGGTGGTCAGGTTTTACATTAAAAACGCTAAAACGGGGGTTGTG |
| shaped_virus_6_-_ | | | GGGGCGTTCCCCCCACAGGGCTCTGGCCCCTGGGACCAGGGTTC |
| NC_013587.1 | | | GAATCCCTGCCCGGCTA (SEQ ID NO: 118) |
| Sulfolobus_spindle- | Gly | CCC | GGGGTGCCCCCCTAATGCCGGCCTCCCAAGCCGGTGATCCCGGG |
| shaped_virus_7_-_ | | | TTCAAATCCCGGCGGCCGCA (SEQ ID NO: 119) |
| NC_013588.1 | | | |
| Sulfolobus_virus_ | Asp | GTC | GGGTGTGGGGGTACCCCCCACCATGGGGGCCTGTCGAGCCCCTG |
| Kamchatka_1_-_ | | | ACCCGGGTTCAAATCCCGGCCGCGGCG (SEQ ID NO: 120) |
| NC_005361.1 | | | |

TABLE 4
Plasmids

| Plasmid | |
|---|---|
| ID | Sequence |
| pLS1 | CGGCTAACTAAGCGGCCTGCTGACTTTCTCGCCGATCAAAAGGCATTTTGCTATTAAGGGATTGACGAGGG |
| | CGATCTGCGCAGTAAGATGCGCCCCGCATTGCACCTATAGCCCAACTGGTAGAGGCAGCAGACTTGAAATC |
| | TGCTCAGTGTGAGTTCGAATCTCTCTAGGTGTACCACTTTCTTCCCGAGCGTGATTGATGGAATTGGCATA |
| | CATACCGTCCTTGAAAGTCGGGTTTTGAGGGTTCGAATCCCTTGTCACGCACCAAATTATGTAAAAAAGCG |
| | CCCTAAAGGCGCTTTTTTACTATCTGCGATACTCAAAGATTCGAACCTGCAGCAGGTTTGAGTTGAGCGCA |
| | GCGAAACAACGGAGCCGCTCGTGCTGGCGACGGCCCGAAGGGCGAGCGAAGCGAGTCATCCTGCACGACCC |
| | ACCAATTCGAAAAGCCTGCTCAACGAGCAGGCTTTTTTGCATGCGGGTCCCCAATAATTACGATTTAAATT |
| | TGACATAAGCCTGTTCGGTTCGTAAACTGTAATGCAAGTAGCGTATGCGCTCACGCAACTGGTCCAGAACC |
| | TTGACCGAACGCAGCGGTGGTAACGGCGCAGTGGCGGTTTTCATGGCTTGTTATGACTGTTTTTTTGTACA |
| | GCCTATGCCTCGGGCATCCAAGCAGCAAGCGCGTTACGCCGTGGGCATAGAATCCAAGCAAGGAGGGAAGA |
| | TTTATGTCACGCTCCTCTAACGATGTTACGCAGCAGGGCTCCCGCCCTAAAACAAAGTCGGGTGGCTCTTC |
| | CATGGGCATCATTCGCACATGTCGTTCAGGCCCTGACCAAGTCAAATCCATGCGGGCTGCTTCAGATTCAT |
| | TCGGTCGTGAGTTCGGAGACGTAGCCACCTACTCCCAACATCAGCCGGACTCCGATTACTCGGGGAACTCA |
| | TCGCGTTCCAAGACATTCATCGCGTCGGCTGCCTTCGACCAAGAAGCGGTTGTTGGCGCTTCAGCGGCTTA |
| | CGTTTCACCCAAGTTTGAGCAGCCGCGTTCTGAGATCTATATCTATGATTCAGCAGTCTCCGGAGAGCACC |
| | GGCGTCAGGGCATTGCCACCGCGTCAATCAATTCGTCAAAGCATGAGGCCAACGCGTCAGGTGCTTATGTG |
| | ATCTACGTGCAAGCAGATTACGGTGACGATCCCGCAGTGGCTTCATATACAAAGTCGGGCATACGGGAAGA |
| | AGTGATGCACTTTGATATCGACCCATCTACCGCCACCTAAAGCGTTGGTAACAGTTACGATACCAGAGAAC |
| | ACTATTGAGGGGAGACCAGAAACAAAAAAAGGCCGCGTTAGCGGCCTTCAATAATTGGTTACTTCACGAGG |
| | GATTGCTTTTCATTGAGAAGAGAAAAGAAAACCGCCGATCCTGTCCACCGCATTACTGCAAGGTAGTGGAC |
| | AAGACCGGCGGTCTTAAGTTTTTTGGCTGAATTTCGAATTTGGTACACCTAGAGAGATTCGAACTCACACT |
| | GAGCAGATTTCGAGTCTGCTGCCTCTACCAGTTGGGCTATAGGTGCATTAAATCTTTTTAGAGAAGTGGTG |
| | CGTGACAAGGGATTCGAACCCTCAAAACCCGACTTTCGAGGACGGTATGTATGCCAATTCCATCAATCACG |
| | CAATAGCGCCGGAAATGCCCGATGGGGGTAATCATTATAAAAGGACCTTCTTGGAGTGTCAACACGAATTT |
| | CTGTCGAGAGCGTCAGTTTCACCTGTTTTACGTAAAAACCCGCTTCGGCGGGTTTTTACTTTTGGAAGGTT |
| | GAGCGACCACTATTCTTCGTAAAAGTTCTGACTGCTGATTGTAATTATTAGAAAAACTCATCCGACATTGA |
| | ATGAAACTGTGATTTATTCATATCAGGATTATCAATACCATATTTTTGAAATGACCGTTTCTGTGAAGAAG |
| | GAGAAAACTCACCTGAGCAGTTCCATGAGATGGCTGAATCCTGGTATCGGTCTGCGATTCCGACTCGTCCA |
| | ACATCAATACAACCTATCGATTTCCCCTCGTCAAAAATCGAGTTATCCGAAGAGAAATCACCATGAGTGAC |
| | GACGGAATCCGGAGAGAATGGTGACGATTTATGCATTTCTTTCCAGACTTGTTCAACAGGCCAGCCATTAC |
| | GCTCGTCATCAAAATCAGACGCATCAACTGAACCGTTATTCATTCGAGATTGCGCCTGAGCTGAACGAAAT |
| | ACGCGGTCAGAGTTAAAAGGACAATTACAAACAGGAATGGAATGCGACCGGCGTGAGAACACTGCTGACGC |
| | ATCAACAATATTTTCACCAGAATCAGGATATTCTTCCGATACCTGGAATGCTGTTTTCCCGGGGATCGCAG |
| | TGGTTGACGACCATGCATCATCAGGAGTACGGATAAAATGCTTGATGGTCGGTGAAGGCATAAATTCCGTC |
| | GACCAGTTTGATCGGACCATCTCATCTGTAACATCATTGGCAACGGAACCTTTGCCATGTTTTGAAAATGA |
| | CTCTGGCGCATCGGGCTTCCCATATGATCGATAGATTGTCGCACCGGATTGCCCGACATTATCGCGAGCCC |
| | ATTTATACCCATACGAATCAGCATCCATGTTGGAATTCGATCGCGGACGAGAGCAAGACGTTTCCCGTTGA |
| | ATATGAGACATTAAACCTCCTTAAGTCCGAATATTATTGAAGCATTTATCAGGGTTATTGTCTCATGAGCG |
| | GATACATATTTGAATGTATTTAGAAAAATAAACAAATAGGGGTTCCGCGAGGTGAAGATCCTTTTTGATAA |
| | TCTCATGACCAAAATCCCTTAACGTGAGTTTTCGTTCCACTGAGCGTCAGACCCCGTAGAAAAGATCAAAG |
| | GATCTTCTTGAGATCCTTTTTTTCTGCGCGTAATCTGCTGCTTGCAAACAAAAAAACCACCGCTACCAGCG |
| | GTGGTTTGTTTGCCGGATCAAGAGCTACCAACTCTTTTTCCGAAGGTAACTGGCTTCAGCAGAGCGCAGAT |
| | ACCAAATACTGTTCTTCTAGTGTAGCCGTAGTTAGGCCACCACTTCAAGAACTCTGTAGCACCGCCTACAT |
| | ACCTCGCTCTGCTAATCCTGTTACCAGTGGCTGCTGCCAGTGGCGATAAGTCGTGTCTTACCGGGTTGGAC |
| | TCAAGACGATAGTTACCGGATAAGGCGCAGCGGTCGGGCTGAACGGGGGGTTCGTGCACACAGCCCAGCTT |
| | GGAGCGAACGACCTACACCGAACTGAGATACCTACAGCGTGAGCTATGAGAAAGCGCCACGCTTCCCGAAG |
| | GGAGAAAGGCGGACAGGTATCCGGTAAGCGGCAGGGTCGGAACAGGAGAGCGCACGAGGGAGCTTCCAGGG |
| | GGAAACGCCTGGTATCTTTATAGTCCTGTCGGGTTTCGCCACCTCTGACTTGAGCGTCGATTTTTGTGATG |
| | CTCGTCAGGGGGGCGGAGCCTATGGAAAAACGCCAGCAACGCGGCCTTTTTACGGTTCCTGGTTACCACCG |
| | TCAAAAAAAACGGCGCTTTTTAGCGCCGTTTTTATTTTTCAACCTTGCATGATCAGTGCA (SEQ ID NO: |
| | 121) |
| PLS2 | CGGCTAACTAAGCGGCCTGCTGACTTTCTCGCCGATCAAAAGGCATTTTGCTATTAAGGGATTGACGAGGG |
| | CGTATCTGCGCAGTAAGATGCGCCCCGCATTGCACCTATAGCCCAACTGGTAGAGGCAGCAGACTTGAAAT |
| | CTACTCAGTGTGAGTTCGAATCTCTCTAGGTGTACCACTTTCTTCCCGAGGGGGTGTAATCGAATTGGCAT |
| | AGGTACTGGACTTGAAATTCAGGTTTTGTGGGTTCGAATCCCACCACCCCTACCAAATTATGTAAAAAAGC |
| | GCCCTAAAGGCGCTTTTTTACTATCTGCGATACTCAAAGATTCGAACCTGCAGCAGGTTTGAGTTGAGCGC |
| | AGCGAAACAACGGAGCCGCTCGTGCTGGCGACGGCCCGAAGGGTGAGCGAAGCGAGTCATCCTGCACGACC |
| | CACCAATTCGAAAAGCCTGCTCAACGAGCAGGCTTTTTTGCATGCGGGTCCCCAATAATTACGATTTAAAT |
| | TTGACATAAGCCTGTTCGGTTCGTAAACTGTAATGCAAGTAGCGTATGCGCTCACGCAACTGGTCCAGAAC |
| | CTTGACCGAACGCAGCGGTGGTAACGGCGCAGTGGCGGTTTTCATGGCTTGTTATGACTGTTTTTTTGTAC |
| | AGCCTATGCCTCGGGCATCCAAGCAGCAAGCGCGTTACGCCGTGGGCATAGAATCCAAGCAAGGAGGGAAG |
| | ATTTATGTCACGCTCCTCTAACGATGTTACGCAGCAGGGCTCCCGCCCTAAAACAAAGTCGGGTGGCTCTT |
| | CCATGGGCATCATTCGCACATGTCGTTCAGGCCCTGACCAAGTCAAATCCATGCGGGCTGCTTCAGATTCA |
| | TTCGGTCGTGAGTTCGGAGACGTAGCCACCTACTCCCAACATCAGCCGGACTCCGATTACTCGGGGAACTC |
| | ATCGCGTTCCAAGACATTCATCGCGTCGGCTGCCTTCGACCAAGAAGCGGTTGTTGGCGCTTCAGCGGCTT |
| | ACGTTTCACCCAAGTTTGAGCAGCCGCGTTCTGAGATCTATATCTATGATTCAGCAGTCTCCGGAGAGCAC |
| | CGGCGTCAGGGCATTGCCACCGCGTCAATCAATTCGTCAAAGCATGAGGCCAACGCGTCAGGTGCTTATGT |
| | GATCTACGTGCAAGCAGATTACGGTGACGATCCCGCAGTGGCTTCATATACAAAGTCGGGCATACGGGAAG |
| | AAGTGATGCACTTTGATATCGACCCATCTACCGCCACCTAAAGCGTTGGTAACAGTTACGATACCAGAGAA |
| | CACTATTGAGGGGAGACCAGAAACAAAAAAAGGCCGCGTTAGCGGCCTTCAATAATTGGTTACTTCACGAG |
| | GGATTGCTTTTCATTGAGAAGAGAAAAGAAAACCGCCGATCCTGTCCACCGCATTACTGCAAGGTAGTGGA |
| | CAAGACCGGCGGTCTTAAGTTTTTTGGCTGAATTTCGAATTTGGTACACCTAGAGAGATTCGAACTCACAC |
| | TGAGCAGATTTCGAGTCTGCTGCCTCTACCAGTTGGGCTATAGGTGCATTAAATCTTTTTAGAGAAGTGGT |
| | AGGGGTGGTGGGATTCGAACCCACAAAACCTGAATTTCGAGTCCAGTACCTATGCCAATTCGATTACACCC |
| | CCAATAGCGCCGGAAATGCCCGATGGGGGTAATCATTATAAAAGGACCTTCTTGGAGTGTCAACACGAATT |
| | TCTGTCGAGAGCGTCAGTTTCACCTGTTTTACGTAAAAACCCGCTTCGGCGGGTTTTTACTTTTGGAAGGT |
| | TGAGCGACCACTATTCTTCGTAAAAGTTCTGACTGCTGATTGTAATTATTAGAAAAACTCATCCGACATTG |
| | AATGAAACTGTGATTTATTCATATCAGGATTATCAATACCATATTTTTGAAATGACCGTTTCTGTGAAGAA |
| | GGAGAAAACTCACCTGAGCAGTTCCATGAGATGGCTGAATCCTGGTATCGGTCTGCGATTCCGACTCGTCC |
| | AACATCAATACAACCTATCGATTTCCCCTCGTCAAAAATCGAGTTATCCGAAGAGAAATCACCATGAGTGA |
| | CGACGGAATCCGGAGAGAATGGTGACGATTTATGCATTTCTTTCCAGACTTGTTCAACAGGCCAGCCATTA |
| | CGCTCGTCATCAAAATCAGACGCATCAACTGAACCGTTATTCATTCGAGATTGCGCCTGAGCTGAACGAAA |
| | TACGCGGTCAGAGTTAAAAGGACAATTACAAACAGGAATGGAATGCGACCGGCGTGAGAACACTGCTGACG |
| | CATCAACAATATTTTCACCAGAATCAGGATATTCTTCCGATACCTGGAATGCTGTTTTCCCGGGGATCGCA |
| | GTGGTTGACGACCATGCATCATCAGGAGTACGGATAAAATGCTTGATGGTCGGTGAAGGCATAAATTCCGT |
| | CGACCAGTTTGATCGGACCATCTCATCTGTAACATCATTGGCAACGGAACCTTTGCCATGTTTTGAAAATG |
| | ACTCTGGCGCATCGGGCTTCCCATATGATCGATAGATTGTCGCACCGGATTGCCCGACATTATCGCGAGCC |
| | CATTTATACCCATACGAATCAGCATCCATGTTGGAATTCGATCGCGGACGAGAGCAAGACGTTTCCCGTTG |
| | AATATGAGACATTAAACCTCCTTAAGTCCGAATATTATTGAAGCATTTATCAGGGTTATTGTCTCATGAGC |
| | GGATACATATTTGAATGTATTTAGAAAAATAAACAAATAGGGGTTCCGCGAGGTGAAGATCCTTTTTGATA |
| | ATCTCATGACCAAAATCCCTTAACGTGAGTTTTCGTTCCACTGAGCGTCAGACCCCGTAGAAAAGATCAAA |
| | GGATCTTCTTGAGATCCTTTTTTTCTGCGCGTAATCTGCTGCTTGCAAACAAAAAAACCACCGCTACCAGC |
| | GGTGGTTTGTTTGCCGGATCAAGAGCTACCAACTCTTTTTCCGAAGGTAACTGGCTTCAGCAGAGCGCAGA |
| | TACCAAATACTGTTCTTCTAGTGTAGCCGTAGTTAGGCCACCACTTCAAGAACTCTGTAGCACCGCCTACA |
| | TACCTCGCTCTGCTAATCCTGTTACCAGTGGCTGCTGCCAGTGGCGATAAGTCGTGTCTTACCGGGTTGGA |
| | CTCAAGACGATAGTTACCGGATAAGGCGCAGCGGTCGGGCTGAACGGGGGGTTCGTGCACACAGCCCAGCT |
| | TGGAGCGAACGACCTACACCGAACTGAGATACCTACAGCGTGAGCTATGAGAAAGCGCCACGCTTCCCGAA |
| | GGGAGAAAGGCGGACAGGTATCCGGTAAGCGGCAGGGTCGGAACAGGAGAGCGCACGAGGGAGCTTCCAGG |
| | GGGAAACGCCTGGTATCTTTATAGTCCTGTCGGGTTTCGCCACCTCTGACTTGAGCGTCGATTTTTGTGAT |
| | GCTCGTCAGGGGGGCGGAGCCTACGGAAAAACGCCAGCAACGCGGCCTTTTTACGGTTCCTGGTTACCACC |
| | GTCAAAAAAAACGGCGCTTTTTAGCGCCGTTTTTATTTTTCAACCTTGCATGATCAGTGCA (SEQ ID NO: |
| | 122) |
| pLS3 | ATGCAGCGCTCTTCCGCTTCCTCGCTCACTGACTCGCTACGCTCGGTCGTTCGACTGCGGCGAGCGGTGTC |
| | AGCTCACTCAAAAGCGGTAATACGGTTATCCACAGAATCAGGGGATAAAGCCGGAAAGAACATGTGAGCAA |
| | AAAGCAAAGCACCGGAAGAAGCCAACGCCGCAGGCGTTTTTCCATAGGCTCCGCCCCCCTGACGAGCATCA |
| | CAAAAATCGACGCTCAAGCCAGAGGTGGCGAAACCCGACAGGACTATAAAGATACCAGGCGTTTCCCCCTG |
| | GAAGCTCCCTCGTGCGCTCTCCTGTTCCGACCCTGCCGCTTACCGGATACCTGTCCGCCTTTCTCCCTTCG |
| | GGAAGCGTGGCGCTTTCTCATAGCTCACGCTGTTGGTATCTCAGTTCGGTGTAGGTCGTTCGCTCCAAGCT |
| | GGGCTGTGTGCACGAACCCCCCGTTCAGCCCGACCGCTGCGCCTTATCCGGTAACTATCGTCTTGAGTCCA |
| | ACCCGGTAAGACACGACTTATCGCCACTGGCAGCAGCCATTGGTAACTGATTTAGAGGACTTTGTCTTGAA |
| | GTTATGCACCTGTTAAGGCTAAACTGAAAGAACAGATTTTGGTGAGTGCGGTCCTCCAACCCACTTACCTT |
| | GGTTCAAAGAGTTGGTAGCTCAGCGAACCTTGAGAAAACCACCGTTGGTAGCGGTGGTTTTTCTTTATTTA |
| | TGAGATGATGAATCAATCGGTCTATCAAGTCAACGAACAGCTATTCCGTTACTCTAGATTTCAGTGCAATT |
| | TATCTCTTCAAATGTAGCACCTGAAGTCACCTGTTGATACCGGGAAGCCCTGGGCCAACTTTTGGCGAAAA |
| | TGAGACGTTGATCGGCACGTAAGAGGTTCCAACTTTCACCATAATGAAATAAGATCACTACCGGGCGTATT |
| | TTTTGAGTTATCGAGATTTTCAGGAGCTAAGGAAGCTAAAATGGAGAAAAAAATCACTGGATATACCACCG |
| | TTGATATATCCCAATGGCATCGTAAAGAACATTTTGAGGCATTTCAGTCCGTTGCTCAATGTACCTATAAC |
| | CAGACCGTTCAGTCAGATATTACGGCCTTTTCAAAGACCGTAAAGAAAAATAAGCACAAGTTTTATCCGGC |
| | CTTTATTCACATTTCGGCCCGCTCGATGAATGCTCATCCGGAATTTCGTATGGCAATGAAAGACGGTGAGT |
| | CGGTGATATGGGATTCTGTTCACCCTTGTTACACCGTTTTCCATGAGCAAACTGAAACGTTTTCCTCTTCA |
| | TGGTCCGAATACCACGACGATTTCCGGCAGTTTTCACACATATATTCCCAAGATGTGGCGTGTTACGGTGA |
| | AAACTCAGCCTATTTCCCTAAAGGGTTTATTGAGAATATGTTTTTCGTCTCCGCCAATCCCTGGGTGTCCT |
| | TCACCTCCTTTGATTCAAACGTGGCCAATATGGACAACTTCTTCGCCCCCGTTTTCACCATGGGCAAATAT |
| | TATACGCAAGGCGACAAGGTGTCAATGCCGTCAGCGATTCAGGTTCATCATGCCGTTTGTGATGGCTTCCA |
| | TGTCGGCCGCATGTCAAATGAATCGCAACAGTACTGCGATGAGTGGCAGGGCGGGGCGTAAAAGGTTGAAA |
| | AATAAAAACGGCGCTAAAAAGCGCCGTTTTTTTTGACGGTGGTATATCAGCGTAACAACGGAAACCGGCCA |
| | TTGCGCCGGTTTTTTTTGGCCT (SEQ ID NO: 123) |
| pLS4 | ATGCAGCGCTCTTCCGCTTCCTCGCTCACTGACTCGCTACGCTCGGTCGTTCGACTGCGGCGAGCGGTGTC |
| | AGCTCACTCAAAAGCGGTAATACGGTTATCCACAGAATCAGGGGATAAAGCCGGAAAGAACATGTGAGCAA |
| | AAAGCAAAGCACCGGAAGAAGCCAACGCCGCAGGCGTTTTTCCATAGGCTCCGCCCCCCTGACGAGCATCA |
| | CAAAAATCGACGCTCAAGCCAGAGGTGGCGAAACCCGACAGGACTATAAAGATACCAGGCGTTTCCCCCTG |
| | GAAGCTCCCTCGTGCGCTCTCCTGTTCCGACCCTGCCGCTTACCGGATACCTGTCCGCCTTTCTCCCTTCG |
| | GGAAGCGTGGCGCTTTCTCATAGCTCACGCTGTTGGTATCTCAGTTCGGTGTAGGTCGTTCGCTCCAAGCT |
| | GGGCTGTGTGCACGAACCCCCCGTTCAGCCCGACCGCTGCGCCTTATCCGGTAACTATCGTCTTGAGTCCA |
| | ACCCGGTAAGACACGACTTATCGCCACTGGCAGCAGCCATTGGTAACTGATTTAGAGGACTTTGTCTTGAA |
| | GTTATGCACCTGTTAAGGCTAAACTGAAAGAACAGATTTTGGTGAGTGCGGTCCTCCAACCCACTTACCTT |
| | GGTTCAAAGAGTTGGTAGCTCAGCGAACCTTGAGAAAACCACCGTTGGTAGCGGTGGTTTTTCTTTATTTA |
| | TGAGATGATGAATCAATCGGTCTATCAAGTCAACGAACAGCTATTCCGTTACTCTAGATTTCAGTGCAATT |
| | TATCTCTTCAAATGTAGCACCTGAAGTCAAGGTGGCACTTTTCGGGGAAATGTGCGCGGAACCCCTATTTG |
| | TTTATTTTTCTAAATACATTCAAATATGTATCCGCTCATGAGACAATAACCCTGATAAATGCTTCAATAAT |
| | ATTCAGGTTACCCTACGATGTCCATTCAACATTTCCGTGTCGCCTCGATTCCCTTTTTTGCGGCATTTTGC |
| | TCGCCTGTTTTTGCTCACCCAGAAACGTCAGTGAAAGTAAAAGATGCTGAAGATCAGTCAGGTGCACGAGT |
| | GGGTTACATCGAATCAGATTCAAACTCTGGTAAGATCTCGGAGTCCTTTCGCCCCGAAGAACGTTTTCCAA |
| | TGATGTCCACTTTTAAAGTTTCGTCATGTGGCGCGGTATCATCCCGTATTGACGCCGGGCAAGAGCAATCG |
| | GGTCGCCGCATACACTATTCTCAGAATGACTCAGTTGAGTACTCCCCAGTCACAGAAAAGCATTCAACGGA |
| | TGGCATGACAGTACGCGAATCGTGCTCTGCTGCCATAACCATGTCTGATAACACTGCGGCCAACTCATCGT |
| | CAACAACGATCGGAGGACCGAAGGAGTCAACCGCTTTTTCACACAACATGGGGGATCATGTAACTCGCTCG |
| | GATCGTTGGGAACCGGAGTCGAATGAAGCCATACCAAACGACGAGCGTGACACCACGATGCCTGTAGCAAT |
| | GGCAACAACGTCGCGCAAATCGTCAACTGGCGAATCGTCAACTTCAGCTTCCCGGCAACAATCAATAGACT |
| | GGATGGAGGCGGATAAAGTTGCAGGACCATCGTCACGCTCCGCCTCACCGGCTGGCTGGTTTATTGCTGAT |
| | AAATCTGGAGCCGGTGAGCGTGGGTCTCGCGGTATCATTGCAGCATCAGGGCCAGATGGTAAGCCCTCCCG |
| | TATCGTAGTTATCTACACGACGGGGTCCCAGGCAACTATGGATGAACGAAATCGCCAGATCGCTGAGATAG |
| | GTGCCTCTTCGATTAAGCATTGGTAATAATCAGCGTAACAACGGAAACCGGCCATTGCGCCGGTTTTTTTT |
| | GGCCT (SEQ ID NO: 124) |
| pLS5 | ATGCAGCGCTCTTCCGCTTCCTCGCTCACTGACTCGCTACGCTCGGTCGTTCGACTGCGGCGAGCGGTGTC |
| | AGCTCACTCAAAAGCGGTAATACGGTTATCCACAGAATCAGGGGATAAAGCCGGAAAGAACATGTGAGCAA |
| | AAAGCAAAGCACCGGAAGAAGCCAACGCCGCAGGCGTTTTTCCATAGGCTCCGCCCCCCTGACGAGCATCA |
| | CAAAAATCGACGCTCAAGCCAGAGGTGGCGAAACCCGACAGGACTATAAAGATACCAGGCGTTTCCCCCTG |
| | GAAGCTCCCTCGTGCGCTCTCCTGTTCCGACCCTGCCGCTTACCGGATACCTGTCCGCCTTTCTCCCTTCG |
| | GGAAGCGTGGCGCTTTCTCATAGCTCACGCTGTTGGTATCTCAGTTCGGTGTAGGTCGTTCGCTCCAAGCT |
| | GGGCTGTGTGCACGAACCCCCCGTTCAGCCCGACCGCTGCGCCTTATCCGGTAACTATCGTCTTGAGTCCA |
| | ACCCGGTAAGACACGACTTATCGCCACTGGCAGCAGCCATTGGTAACTGATTTAGAGGACTTTGTCTTGAA |
| | GTTATGCACCTGTTAAGGCTAAACTGAAAGAACAGATTTTGGTGAGTGCGGTCCTCCAACCCACTTACCTT |
| | GGTTCAAAGAGTTGGTAGCTCAGCGAACCTTGAGAAAACCACCGTTGGTAGCGGTGGTTTTTCTTTATTTA |
| | TGAGATGATGAATCAATCGGTCTATCAAGTCAACGAACAGCTATTCCGTTACTCTAGATTTCAGTGCAATT |
| | TATCTCTTCAAATGTAGCACCTGAAGTCAGGGTCCCCAATAATTACGATTTAAATTTGACATAAGCCTGTT |
| | CGGTTCGTAAACTGTAATGCAAGTAGCGTATGCGCTCACGCAACTGGTCCAGAACCTTGACCGAACGCAGC |
| | GGTGGTAACGGCGCAGTGGCGGTTTTCATGGCTTGTTATGACTGTTTTTTTGTACAGCCTATGCCTCGGGC |
| | ATCCAAGCAGCAAGCGCGTTACGCCGTGGGCATAGAATCCAAGCAAGGAGGGAAGATTTATGTCACGCTCC |
| | TCTAACGATGTTACGCAGCAGGGCTCCCGCCCTAAAACAAAGTCGGGTGGCTCTTCCATGGGCATCATTCG |
| | CACATGTCGTTCAGGCCCTGACCAAGTCAAATCCATGCGGGCTGCTTCAGATTCATTCGGTCGTGAGTTCG |
| | GAGACGTAGCCACCTACTCCCAACATCAGCCGGACTCCGATTACTCGGGGAACTCATCGCGTTCCAAGACA |
| | TTCATCGCGTCGGCTGCCTTCGACCAAGAAGCGGTTGTTGGCGCTTCAGCGGCTTACGTTTCACCCAAGTT |
| | TGAGCAGCCGCGTTCTGAGATCTATATCTATGATTCAGCAGTCTCCGGAGAGCACCGGCGTCAGGGCATTG |
| | CCACCGCGTCAATCAATTCGTCAAAGCATGAGGCCAACGCGTCAGGTGCTTATGTGATCTACGTGCAAGCA |
| | GATTACGGTGACGATCCCGCAGTGGCTTCATATACAAAGTCGGGCATACGGGAAGAAGTGATGCACTTTGA |
| | TATCGACCCATCTACCGCCACCTAAAAGGTTGAAAAATAAAAACGGCGCTAAAAAGCGCCGTTTTTTTTGA |
| | CGGTGGTATATCAGCGTAACAACGGAAACCGGCCATTGCGCCGGTTTTTTTTGGCCT (SEQ ID NO: 125) |
| pLS6 | CCAAAAGTAAAAACCCGCCGAAGCGGGTTTTTACGTAAAACAGGTGAAACTGACTTACGCCCCGCCCTGCC |
| | ACTCATCGCAGTACTGTTGCGATTCATTTGACATGCGGCCGACATGGAAGCCATCACAAACGGCATGATGA |
| | ACCTGAATCGCTGACGGCATTGACACCTTGTCGCCTTGCGTATAATATTTGCCCATGGTGAAAACGGGGGC |
| | GAAGAAGTTGTCCATATTGGCCACGTTTGAATCAAAGGAGGTGAAGGACACCCAGGGATTGGCGGAGACGA |
| | AAAACATATTCTCAATAAACCCTTTAGGGAAATAGGCTGAGTTTTCACCGTAACACGCCACATCTTGGGAA |
| | TATATGTGTGAAAACTGCCGGAAATCGTCGTGGTATTCGGACCATGAAGAGGAAAACGTTTCAGTTTGCTC |
| | ATGGAAAACGGTGTAACAAGGGTGAACAGAATCCCATATCACCGACTCACCGTCTTTCATTGCCATACGAA |
| | ATTCCGGATGAGCATTCATCGAGCGGGCCGAAATGTGAATAAAGGCCGGATAAAACTTGTGCTTATTTTTC |
| | TTTACGGTCTTTGAAAAGGCCGTAATATCTGACTGAACGGTCTGGTTATAGGTACATTGAGCAACGGACTG |
| | AAATGCCTCAAAATGTTCTTTACGATGCCATTGGGATATATCAACGGTGGTATATCCAGTGATTTTTTTCT |
| | CCATTTTAGCTTCCTTAGCTCCTGAAAATCTCGATAACTCAAAAAATACGCCCGGTAGTGATCTTATTTCA |
| | TTATGGTGAAAGTTGGAACCTCTTACGTGCCGATCAACGTCTCATTTTCGCCAAAAGTTGGCCCAGGGCTT |
| | CCCGGTATCAACAGGTCCATGGGTATGGACAGTTTTCCCTTTGATATGTAACGGTGAACAGTTGTTCTACT |
| | TTTGTTTGTTAGTCTTGATGCTTCACTGATAGATACAAGAGCCATAAGAACCTCAGATACGTCCGTATTTA |
| | GCTGATATGTTCTCCGATGTGGTTCGTTGTTTTTGCGTTGACCAGGAGAAGGAACCATTTGAATCATACTT |
| | GGATTGCATGTCAGACGAAAATTTTGCCTCAAAAGAGGTTGACTGAATTTTTGCAGTTGAAGCATCGTGCG |
| | ATGTTTTACGTGATCCGTTACGTGAGTAGGAATCAGATGTAATGGTTGTTGGTATTTTGTCACCATTCATT |
| | TTTATCTGGTTGTTCTCTGATTCGGTTACTGAATCCATTTGTCGATCCGATTCAACTTGGAAAATCGACGT |
| | ATCAGTCGGGCGGCCTCGCTTATCAACCACCGATTTCATATTAGAGTAAGTGTTCGAATCTTTAGATATTG |
| | GTTTTGAAACCCATTGGTTTGAGCGTTTAAACTCATGGTAGTTATTTTCTGACATCGACATGAACTTAAAT |
| | TCATCTGAGGAAATCTCTATATTTGCCTTGTGAGTTTTCTTTTGTGTCGATTCTTTCGATGACCACTCATA |
| | AATCCGCATAGAGTATTTGTTTTCAAAAGACTTAACATGTTCTGAATTATATTTTATGAATTTTTTCGACT |
| | GGAATGAATAAGGTGATATCTCTTCAGAAAAAACCGATTCCGATTTTTCAGAGGAGAACTTGGCATAGTTT |
| | GTCCACTGGAAAATCTCAAAGCCTTTAACTGAAGGATTACGGATTTCCACAGTACGCGTCATTGACTCACG |
| | GGTTGCTTTAGCTGATACACCATAAGCATTTTCACGAGAGATGTTCATCATCTGAGCGTATTGGTTATAAG |
| | TGAAAGATACCGTCCGTTCTTTACGTGTAGGGTTTTCAATCGTGGGGTTCGATGATGCCACACAGCACGAA |
| | ATCGACTTGGTTTCATGCTCCGTTGAGTCATAGCGAGAAATCGCCGATTCATTTGCTTTGAAAACAACCGA |
| | TTCAGACATACATCTCAATTGGTCTAGGTGATTTTAATCACTATACCAATTGAGATGGGCTAGTCAATGAT |
| | AATTACTAGTCCTTTTCCTTTGAGTTGTGGGTATCTGTAAATTCTGCTAGACCTTTGCTGGAAAACTTGTA |
| | AATTCTGCTAGACCCTCTGTAAATTCCGCTAGACCTTTGTGTGTTTTTTTTGTTTATATTCAAGTGGTTAT |
| | AATTTATAGAATAAAGAAAGAATAAAAAAAGATAAAAAGAATAGATCCCAGCCCTGTGTATAACTCACTAC |
| | TTTAGTCAGTTCCGCAGTATTACAAAAGGATGTCGCAAACGCTGTTTGCTCCTCTACAAAACAGACCTTAA |
| | AACCCTAAAGGCTTAAGTAGCACCCTCGCAAGCTCGGTTGCGGCCGCAATCGGGCAAATCGCTGAATATTC |
| | CTTTTGTCTCCGACCATCAGGCACCTGAGTCGCTGTCTTTTTCGTGACATTCAGTTCGCTGCGCTCACGGC |
| | TCTGGCAGTGAATGGGGGTAAATGGCACTACAGGCGCCTTTTATGGATTCATGCAAGGAAACTACCCATAA |
| | TACAAGAAAAGCCCGTCACGGGCTTCTCAGGGCGTTTTATGGCGGGTCTGCTATGTGGTGCTATCTGACTT |
| | TTTGCTGTTCAGCAGTTCCTGCCCTCTGATTTTCCAGTCTGACCACTTCGGATTATCCCGTGACAGGTCAT |
| | TCAGACTGGCTAATGCACCCAGTAAGGCAGCGGTATCATCAACGGGGTCTGACGCTCAGTGGAACG (SEQ |
| | ID NO: 126) |
| pLS7 | TTACGCCCCGCCCTGCCACTCATCGCAGTACTGTTGCAGTTCATTAAGCATGCGGCCGACATGGAAGCCAT |
| | CACAAACGGCATGATGAACCTGAATCGCCAGCGGCATCAGCACCTTGTCGCCTTGCGTATAATATTTGCCC |
| | ATGGTGAAAACGGGGGCGAAGAAGTTGTCCATATTGGCCACGTTGAGATCAAAGGAGGTGAAGGACACCCA |
| | GGGATTGGCGGAGACGAAAAACATATTCTCAATAAACCCTTTAGGGAAATAGGCCAGGTTTTCACCGTAAC |
| | ACGCCACATCTTGGGAATATATGTGTAGAAACTGCCGGAAATCGTCGTGGTATTCGGACCAGAGAGAGGAA |
| | AACGTTTCAGTTTGCTCATGGAAAACGGTGTAACAAGGGTGAACAGAATCCCATATCACCAGCTCACCGTC |
| | TTTCATTGCCATACGAAATTCCGGATGAGCATTCATCAGGCGGGCAAGAATGTGAATAAAGGCCGGATAAA |
| | ACTTGTGCTTATTTTTCTTTACGGTCTTCAGAAAGGCCGTAATATCCAGCTGAACGGTCTGGTTATAGGTA |
| | CATTGAGCAACGGACTGAAATGCCTCAAAATGTTCTTTACGATGCCATTGGGATATATCAACGGTGGTATA |
| | TCCAGTGATTTTTTTCTCCATTTTAGCTTCCTTAGCTCCTGAAAATCTCGATAACTCAAAAAATACGCCCG |
| | GTAGTGATCTTATTTCATTATGGTGAAAGTTGGAACCTCTTACGTGCCGATCAACGTCTCATTTTCGCCAA |
| | AAGTTGGCCCAGGGCTTCCCGGTATCAACAGGTACCGCCTTTGAGTGAGCTGATACCGCTCGCCGCAGTAG |
| | AAAAGATCAAAGGATCTTCTTGAGATCCTTTTTTTCTGCGGGGGATCAGGACCGCTGCCGGAGCGCAACCC |
| | ACTCACTACAGCAGAGCCATGTAGGGCCGCCGGCGTTGTGGATACCTCGCGGAAAACTTGGCCCTCACTGA |
| | CAGATGAGGGGCGGACGTTGACACTTGAGGGGCCGACTCACCCGGCGCGGCGTTGACAGATGAGGGGCAGG |
| | CTCGATTTCGGCCGGCGACGTGGAGCTGGCCAGCCTCGCAAATCGGCGAAAACGCCTGATTTTACGCGAGT |
| | TTCCCACAGATGATGTGGACAAGCCTGGGGATAAGTGCCCTGCGGTATTGACACTTGAGGGGCGCGACTAC |
| | TGACAGATGAGGGGCGCGATCCTTGACACTTGAGGGGCAGAGTGCTGACAGATGAGGGGCGCACCTATTGA |
| | CATTTGAGGGGCTGTCCACAGGCAGAAAATCCAGCATTTGCAAGGGTTTCCGCCCGTTTTTCGGCCACCGC |
| | TAACCTGTCTTTTAACCTGCTTTTAAACCAATATTTATAAACCTTGTTTTTAACCAGGGCTGCGCCCTGTG |
| | CGCGTGACCGCGCACGCCGAAGGGGGGTGCCCCCCCTTCTCGAACCCTCCCGGCCCGCTAACGCGGGCCTC |
| | CCATCCCCCCAGGGGCTGCGCCCCTCGGCCGCGAACGGCCTCACCCCAAAAATGGAGGTTTCTCTTCTTTC |
| | ACTTTACGGCTAGCTCAGCCCTAGGTATTATGCTAGCAGCCCCATACGATATAAGGAGGGTGAATGAATCG |
| | GACGTTTGACCGGAAGGCATACCGACAAGAATCAATCGACGCGGGGTTTTCCGCCGAGGATGCCGAAACCA |
| | TCGCATCTCGCACCGTCATGCGTGCGCCCCGCGAAACCTTCCAGTCCGTCGGCTCTATAGTCCAGCAAGCT |
| | ACGGCCAAGATCGAGCGCGACTCTGTGCAATCGGCTCCCCCTGCCTCGCCCGCGCCATCTGCCGCCGTGGA |
| | GCGTTCTCGTCGTTCGGAACAGGAGGCGGCAGGTTCGGCGAAGTCTATGACCATCGATACACGAGGTACTA |
| | TGACGACCAAGAAGCGAAAAACCGCCGGCGAGGACTCAGCAAAACAGGTCTCTGAAGCCAAGCAGGCCGCG |
| | TCGTCAAAACACACGAAGCAGCAGATCAAGGAAATGCAGTCATCCTCATTCGATATTGCGCCGTGGCCGGA |
| | CACGATGCGAGCGATGCCAAACGACACGGCCCGCTCTGCCTCGTTCACCACGCGCAACAAGAAAATCCCGC |
| | GCGAGGCGTCACAAAACAAGGTCATTTTCCACGTCAACAAGGACGTGAAGATCACCTACACCGGCGTCGAG |
| | TCACGGGCCGACGATGACGAATCGGTGTGGCAGCAGGTGTCAGAGTACGCGAAGCGCACCCCTATCGGCGA |
| | GCCGATCACCTTCACGTTCTACGAGTCATGCCAGGACTCGGGCTGGTCCATCAATGGCCGGTATTACACGA |
| | AGGCCGAGGAATGCTCATCTCGCTCACAGGCGACGGCGATGGGCTTCACGTCCGACCGCGTTGGGCACTCA |
| | GAATCTGTGTCTTCATCGCACCGCTTCCGCGTCTCGGACCGTGGCAAGAAAACGTCCCGTTGCCAGGTCTC |
| | GATCGACGAGGAAATCGTCGTGTCATTTGCTGGCGACCACTACACGAAATTCATATGGGAGAAGTACCGCA |
| | AGTCATCTCCGACGGCCCGACGGATGTTCGACTATTTCTCTTCCCACCGGGAGCCGTACCCGTCAAAGTCA |
| | GAAACCTTCCGCTCAATGTGCGGATCTGATTCCACCCGCGTGAAGAAGTGGCGCGAGCAGGTCGGCGAAGC |
| | CTGCGAAGAGTCACGAGGCTCCGGCTCAGTGGAACACGCCTGGGTCAATGATGACTCGGTGCATTGCAAAC |
| | GCTAATACCTCGCTCTGCTAATCCTGTTACCAGTGGCTGCTACCACCGTCAAAAAAAACGGCGCTTTTTAG |
| | CGCCGTTTTTATTTTTCAACCTT (SEQ ID NO: 127) |

What is claimed is:

1. A method of conferring viral resistance to a cell, comprising:
delivering to the cell an engineered nucleic acid encoding the first viral tRNA variant, wherein the first viral tRNA variant binds to a first messenger ribonucleic acid (mRNA) codon and is charged with an amino acid not encoded by the first mRNA codon.

2. A method of conferring viral resistance to a cell, comprising:
delivering to the cell a first viral transfer ribonucleic acid (tRNA) variant, wherein the first viral tRNA variant binds to a first messenger ribonucleic acid (mRNA) codon and is charged with an amino acid not encoded by the first mRNA codon.

3. The method of claim 1 or 2, wherein the cell comprises a second viral tRNA variant, wherein the second viral tRNA variant binds to a second mRNA codon and is charged with an amino acid not encoded by the second mRNA codon, and wherein the second mRNA codon is different from the first mRNA codon.

4. The method of claim 3, further comprising delivering to the cell the second viral tRNA variant or an engineered nucleic acid encoding the second viral tRNA variant.

5. The method of any one of the preceding claims, wherein the first mRNA codon encodes an amino acid selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

6. The method of claim 5, wherein the first mRNA codon encodes a serine, optionally wherein the first mRNA codon is selected from UCA, UCG, UCC, and UCU, preferably UCA and UCG.

7. The method of any one of claims 3-6, wherein the second mRNA codon encodes an amino acid selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

8. The method of claim 7, wherein the second mRNA codon encodes a serine, optionally wherein the second mRNA codon is selected from UCA, UCG, UCC, and UCU, preferably UCA and UCG.

9. The method of any one of the preceding claims, wherein the first amino acid is selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine, optionally wherein the first amino acid is leucine.

10. The method of any one of the preceding claims, wherein the second amino acid is selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine, optionally wherein the second amino acid is leucine.

11. The method of any one of the preceding claims, wherein the cell does not comprise an endogenous tRNA that binds to the first mRNA codon.

12. The method of any one of claims 3-11, wherein the cell does not comprise an endogenous tRNA that binds to the second mRNA codon.

13. The method of any one of the preceding claims, wherein the cell expresses an essential gene encoding a protein that comprises a nonstandard amino acid.

14. The method of claim 13, wherein the cell comprises a third tRNA, wherein the third tRNA binds to a natural or non-natural amino acid and is charged with the nonstandard amino acid.

15. The method of claim 14, further comprising delivering to the cell an engineered nucleic acid encoding the third tRNA.

16. The method of any one of claims 13-15, wherein the nonstandard amino acid is L-4,4′-biphenylalanine (bipA).

17. The method of any one of the preceding claims, wherein the cell is a mammalian cell, a yeast cell, a plant cell, or a bacterial cell.

18. The method of any one of the preceding claims, wherein the cell further comprises an mRNA comprising the first mRNA codon to which the first viral tRNA variant binds.

19. The method of claim 18, wherein the mRNA further comprises the second mRNA codon to which the second viral tRNA variant binds.

20. The method of any one of the preceding claims, wherein the cell comprises an artificial genetic code in which an mRNA codon encodes an amino acid different from its natural amino acid identity.

21. The method of claim 20 further comprising delivering to the cell a vector (e.g., plasmid) comprising a gene of interest, wherein the vector can only be expressed in the cell comprising the artificial genetic code (i.e., cannot be expressed in a cell comprising a canonical genetic code), optionally wherein the vector is a plasmid comprising a sequence selected from Table 4 or a sequence having at least 80%, at least 85%, at least 90%, or at least 95% identity to a sequence selected from Table 4.

22. The method of any one of the preceding claims, wherein the first and/or second viral tRNA variant comprises the sequence of any one of SEQ ID NO: 1-6 or is selected from the tRNA variants of Table 3.

23. A cell, comprising:
an engineered nucleic acid encoding the first viral tRNA variant, wherein the first viral tRNA variant binds to a first messenger ribonucleic acid (mRNA) codon and is charged with an amino acid not encoded by the first mRNA codon, wherein the cell is resistant to viral infection.

24. An engineered cell, comprising:
a first viral transfer ribonucleic acid (tRNA) variant, wherein the first viral tRNA variant binds to a first messenger ribonucleic acid (mRNA) codon and is charged with an amino acid not encoded by the first mRNA codon, wherein the cell is resistant to viral infection.

25. The cell of claim 24, wherein the cell comprises second viral tRNA variant, wherein the second viral tRNA variant binds to a second mRNA codon and is charged with an amino acid not encoded by the second mRNA codon, and wherein the second mRNA codon is different from the first mRNA codon.

26. The cell of claim 25, wherein the cell comprises an engineered nucleic acid encoding the second viral tRNA variant.

27. The cell of any one of the preceding claims, wherein the first mRNA codon encodes an amino acid selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

28. The cell of claim 27, wherein the first mRNA codon encodes a serine, optionally wherein the first mRNA codon is selected from UCA, UCG, UCC, and UCU, preferably UCA and UCG.

29. The cell of any one of claims 25-28, wherein the second mRNA codon encodes an amino acid selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine.

30. The cell of claim 29, wherein the second mRNA codon encodes a serine, optionally wherein the second mRNA codon is selected from UCA, UCG, UCC, and UCU, preferably UCA and UCG.

31. The cell of any one of the preceding claims, wherein the first amino acid is selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine, optionally wherein the first amino acid is leucine.

32. The cell of any one of the preceding claims, wherein the second amino acid is selected from alanine, cysteine, aspartic acid, glutamic acid, phenylalanine, glycine, histidine, isoleucine, lysine, leucine, methionine, asparagine, proline, glutamine, arginine, serine, threonine, tryptophan, tyrosine, and valine, optionally wherein the second amino acid is leucine.

33. The cell of any one of the preceding claims, wherein the cell does not comprise an endogenous tRNA that binds to the first mRNA codon.

34. The cell of any one of claims 25-33, wherein the cell does not comprise an endogenous tRNA that binds to the second mRNA codon.

35. The cell of any one of the preceding claims, wherein the cell expresses an essential gene encoding a protein that comprises a nonstandard amino acid.

36. The cell of claim 35, wherein the cell further comprises tRNA that binds to a codon encoding a natural or non-natural amino acid (e.g., a TAG stop codon) and is charged with the nonstandard amino acid.

37. The cell of claim 36, wherein the cell further comprises an engineered nucleic acid encoding the tRNA.

38. The cell of any one of claims 35-37, wherein the nonstandard amino acid is L-4,4′-biphenylalanine (bipA).

39. The cell of any one of the preceding claims, wherein the cell is a mammalian cell, a yeast cell, a plant cell, or a bacterial cell.

40. The cell of any one of the preceding claims, wherein the cell further comprises an mRNA comprising the first mRNA codon to which the first viral tRNA variant binds.

41. The cell of claim 40, wherein the mRNA further comprises the second mRNA codon to which the second viral tRNA variant binds.

42. The cell of any one of the preceding claims comprising an artificial genetic code in which an mRNA codon encodes an amino acid different from its natural amino acid identity.

43. The cell of claim 42 further comprising a vector (e.g., plasmid) comprising a gene of interest, wherein the vector can only be expressed in the cell comprising the artificial genetic code (i.e., cannot be expressed in a cell comprising a canonical genetic code), optionally wherein the vector is a plasmid comprising a sequence selected from Table 4 or a sequence having at least 80%, at least 85%, at least 90%, or at least 95% identity to a sequence selected from Table 4.

44. The cell of any one of the preceding claims, wherein the first and/or second viral tRNA variant comprises the sequence of any one of SEQ ID NOs: 1-6 or is selected from the tRNA variants of Table 3.
